(19) United States
(12) Patent Application Publication
(10) Pub. No.: US 2020/0247790 A1
(43) Pub. Date: Aug. 6, 2020

(54) AMINE-SUBSTITUTED HETEROCYCLIC COMPOUNDS AS EHMT2 INHIBITORS, SALTS THEREOF, AND METHODS OF SYNTHESIS THEREOF

(71) Applicant: Epizyme, Inc., Cambridge, MA (US)

(72) Inventors: John Emmerson CAMPBELL, Cambridge, MA (US); Kenneth William DUNCAN, Westwood, MA (US); James Edward John MILLS, Kent (GB); Michael John MUNCHHOF, Corvallis, MT (US)

(21) Appl. No.: 16/756,565

(22) PCT Filed: Oct. 18, 2018
(86) PCT No.: PCT/US2018/056428

§ 371 Date: Apr. 16, 2020

Related U.S. Application Data
(60) Provisional application No. 62/573,917, filed on Oct. 18, 2017.

(51) Int. Cl. C07D 405/14; C07D 401/14; C07D 403/12

(52) U.S. Cl. CPC C07D 405/14; C07D 403/12; C07D 401/14

(57) ABSTRACT

The present disclosure relates to amine-substituted heterocyclic compounds. The present disclosure also relates to pharmaceutical compositions containing these compounds and methods of treating a disorder (e.g., cancer) by administering an amine-substituted heterocyclic compound disclosed herein or a pharmaceutical composition thereof to subjects in need thereof. The present disclosure also relates to the use of such compounds for research or other non-therapeutic purposes.

### RELATED APPLICATION

This application claims benefit of, and priority to, U.S. Application No. 62/573,917, filed on Oct. 18, 2017, the entire content of which is incorporated herein by reference.

### BACKGROUND

Methylation of protein lysine residues is an important signaling mechanism in eukaryotic cells, and the methylation state of histone lysines encodes signals that are recognized by a multitude of proteins and protein complexes in the context of epigenetic gene regulation.

Histone methylation is catalyzed by histone methyltransferases (HMTs), and HMTs have been implicated in various human diseases. HMTs can play a role in either activating or repressing gene expression, and certain HMTs (e.g., euchromatic histone-lysine N-methyltransferase 2 or EHMT2, also called G9a) may methylate many nonhistone proteins, such as tumor suppressor proteins (see, e.g., Liu et al., Journal of Medicinal Chemistry 56:8931-8942, 2013 and Krivega et al., Blood 126(5):665-672, 2015).

Two related HMTs, EHMT1 and EHMT2, are overexpressed or play a role in diseases and disorders such as sickle cell anemia (see, e.g., Renneville et al., Blood 126(16): 1930-1939, 2015) and proliferative disorders (e.g., cancers), and other blood disorders.

### SUMMARY

In one aspect, the present disclosure provides, inter alia, compounds selected from the group consisting of

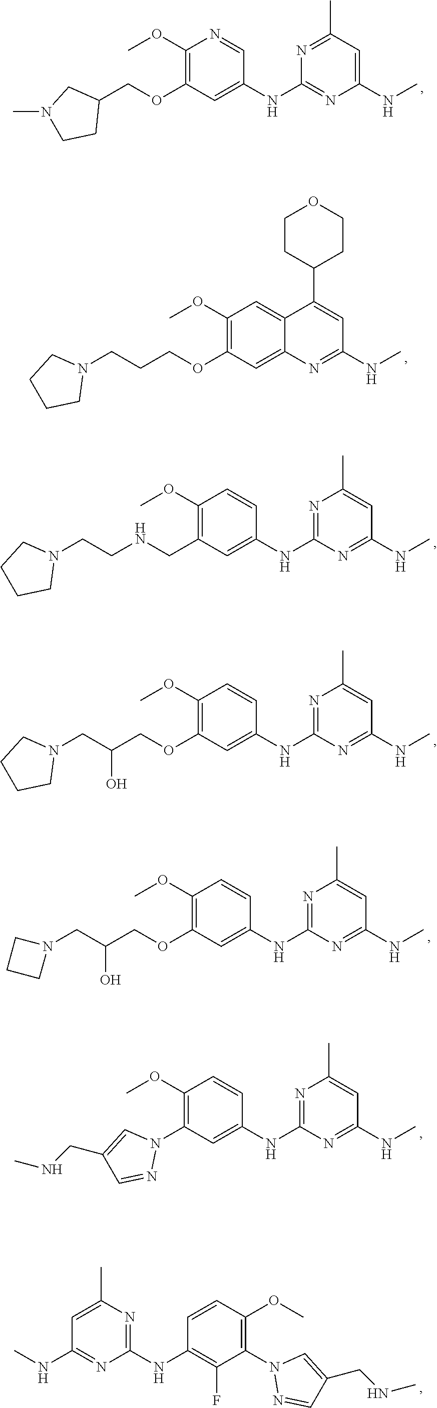

tautomers thereof, pharmaceutically acceptable salts thereof, and pharmaceutically acceptable salts of the tautomers.

In some aspects, the present disclosure features pharmaceutical compositions comprising one or more pharmaceutically acceptable carriers and one or more of the compounds of the present disclosure.

In some aspects, the present disclosure features a method of inhibiting one or more HMTs (e.g., EHMT1 and/or EHMT2). The method includes administering to a subject in need thereof a therapeutically effective amount of a compound of the present disclosure, or a tautomer thereof, or a pharmaceutically acceptable salt of the compound or the tautomer. In some embodiments, the subject has one or more disorders associated with the activity of one or more HMTs (e.g., EHMT1 and/or EHMT2), thereby benifiting from the inhibition of one or more HMTs (e.g., EHMT1 and/or EHMT2). In some embodiments, the subject has an EHMT-mediated disorder. In some embodiments, the subject has a disease, disorder, or condition that is mediated at least in part by the activity of one or both of EHMT1 and EHMT2.

In some aspects, the present disclosure features a method of preventing or treating an EHMT-mediated disorder. The method includes administering to a subject in need thereof a therapeutically effective amount of a compound of the present disclosure, or a tautomer thereof, or a pharmaceutically acceptable salt of the compound or the tautomer. The EHMT-mediated disorder is a disease, disorder, or condition that is mediated at least in part by the activity of EHMT1 or EHMT2 or both. In some embodiments, the EHMT-mediated disorder is a blood disease or disorder. In some embodiments, the EHMT-mediated disorder is selected from proliferative disorders (e.g., cancers such as leukemia, hepatocellular carcinoma, prostate carcinoma, and lung cancer), addiction (e.g., cocaine addiction), and mental retardation.

In some embodiments, the EHMT-mediated disease or disorder comprises a disorder that is associated with gene silencing by one or more HMTs (e.g., EHMT1 and/or EHMT2). In some embodiments, EHMT-mediated disease or disorder is a blood disease or disorder associated with gene silencing by EHMT2.

In some embodiments, the method comprises the step of administering to a subject having a disease or disorder associated with gene silencing by one or more HMTs (e.g., EHMT1 and/or EHMT2) a therapeutically effective amount of one or more compounds of the present disclosure, wherein the compound(s) inhibits histone methyltransferase activity of one or more HMTs (e.g., EHMT1 and/or EHMT2), thereby treating the disease or disorder.

In some embodiments, the blood disease or disorder is selected from the group consisting of sickle cell anemia and beta-thalassemia.

In some embodiments, the blood disease or disorder is hematological cancer.

In some embodiments, the hematological cancer is acute myeloid leukemia (AML) or chronic lymphocytic leukemia (CLL).

In some embodiments, the method further comprises the steps of performing an assay to detect the degree of histone methylation by one or more HMTs (e.g., EHMT1 and/or EHMT2) in a sample comprising blood cells from a subject in need thereof. In some embodiments, performing the assay to detect methylation of H3-K9 in the histone substrate comprises measuring incorporation of labeled methyl groups. In some embodiments, the labeled methyl groups are isotopically labeled methyl groups. In some embodiments, performing the assay to detect methylation of H3-K9 in the histone substrate comprises contacting the histone substrate with an antibody that binds specifically to dimethylated H3-K9.

Unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models. Methods described herein may be used to identify suitable candidates for treating or preventing EHMT-mediated disorders. For example, the disclosure also provides methods of identifying an inhibitor of EHMT1 or EHMT2 or both.

In some aspects, the present disclosure features a method of inhibiting conversion of H3-K9 to dimethylated H3-K9. The method comprises the step of contacting a mutant EHMT, the wild-type EHMT, or both, with a histone substrate comprising H3-K9 and an effective amount of a compound of the present disclosure, wherein the compound inhibits histone methyltransferase activity of EHMT, thereby inhibiting conversion of H3-K9 to dimethylated H3-K9.

In some aspects, the present disclosure features compounds disclosed herein for use in inhibiting one or both of EHMT1 and EHMT2 in a subject in need thereof.

In some aspects, the present disclosure features compounds disclosed herein for use in preventing or treating an EHMT-mediated disorder in a subject in need thereof.

In some aspects, the present disclosure features compounds disclosed herein for use in preventing or treating a blood disorder in a subject in need thereof.

In some aspects, the present disclosure features compounds disclosed herein for use in preventing or treating a cancer in a subject in need thereof.

In some aspects, the present disclosure features use of a compound of the present disclosure in the manufacture of a medicament for inhibiting one or both of EHMT1 and EHMT2 in a subject in need thereof.

In some aspects, the present disclosure features use of a compound of the present disclosure in the manufacture of a medicament for preventing or treating an EHMT-mediated disorder in a subject in need thereof.

In some aspects, the present disclosure features use of a compound of the present disclosure in the manufacture of a medicament for preventing or treating a blood disorder in a subject in need thereof.

In some aspects, the present disclosure features use of a compound of the present disclosure in the manufacture of a medicament for preventing or treating a cancer in a subject in need thereof.

Further, the compounds or methods described herein can be used for research (e.g., studying epigenetic enzymes) and other non-therapeutic purposes.

In some embodiments, the compounds of the present disclosure do not show significant inhibitory activity towards a kinase. Absence of significant kinase inhibition can be determined by measuring IC50 values for one or more kinases of interest, wherein IC50 values greater than a certain reference value are indicative of low or no inhibitory activity towards a given kinase. For example, in some embodiment, the compounds of the present disclosure inhibit a kinase with an enzyme inhibition IC₅₀ value of about 100 nM or greater, 1 μM or greater, 10 μM or greater, 100 μM or greater, or 1000 μM or greater.

In some embodiments, one or more of the compounds of the present disclosure inhibit a kinase with an enzyme inhibition IC₅₀ value of about 1 mM or greater.

In some embodiments, one or more of the compounds of the present disclosure inhibit a kinase with an enzyme inhibition IC₅₀ value of 1 μM or greater, 2 μM or greater, 5 μM or greater, or 10 μM or greater, wherein the kinase is one or more of the following: AbI, AurA, CHK1, MAP4K, IRAK4, JAK3, EphA2, FGFR3, KDR, Lck, MARK1, MNK2, PKCb2, SIK, and Src.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTIONS OF FIGURES

FIG. 1A shows XRPD pattern of Compound 1R freebase Type A.

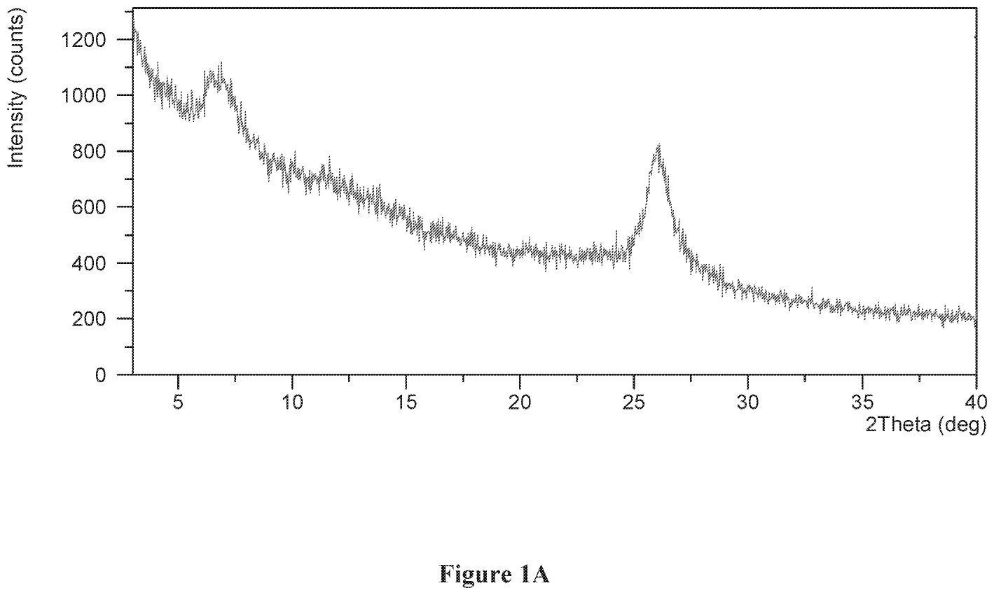

FIG. 1B shows XRPD pattern of Compound 1R freebase Type B.

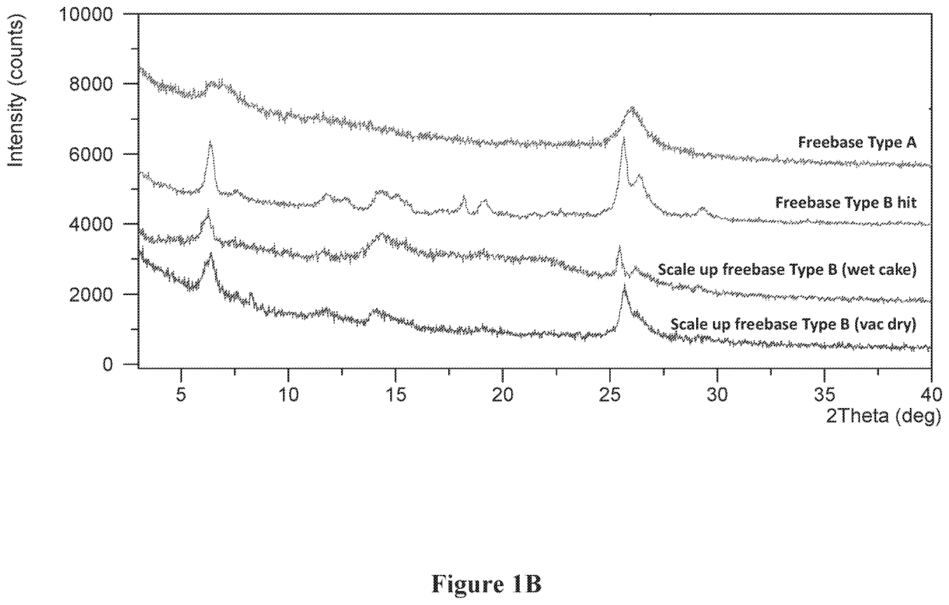

FIG. 2A shows XRPD pattern of Compound 2 freebase Type A.

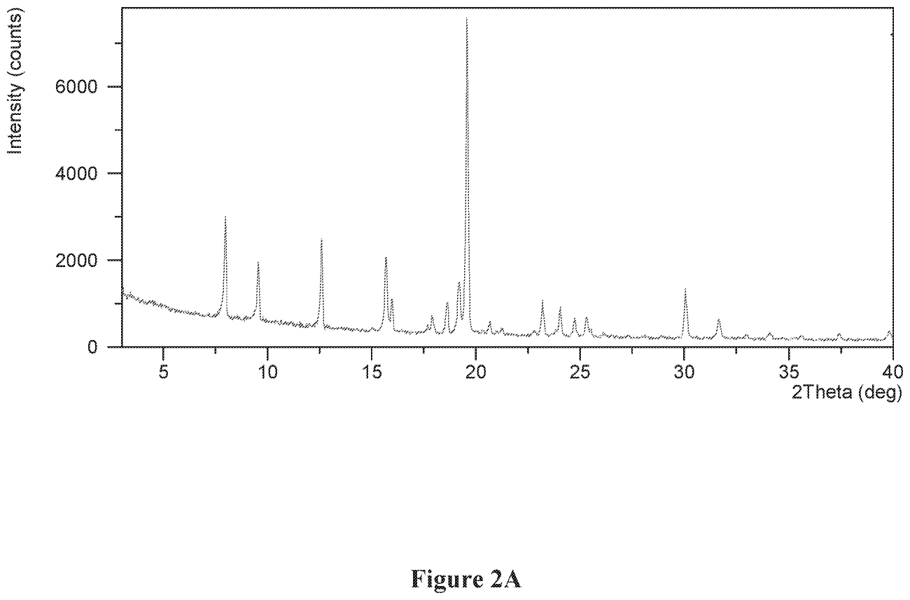

FIG. 2B shows XRPD overlay of Compound 2 freebase Type A before and after DVS.

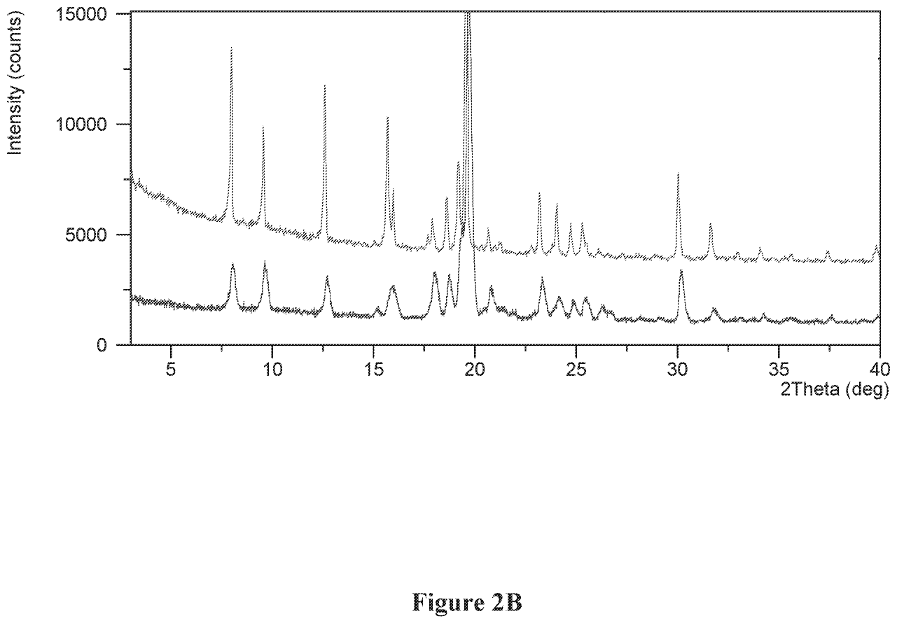

FIG. 3 shows XRPD pattern of Compound 3 freebase Type A.

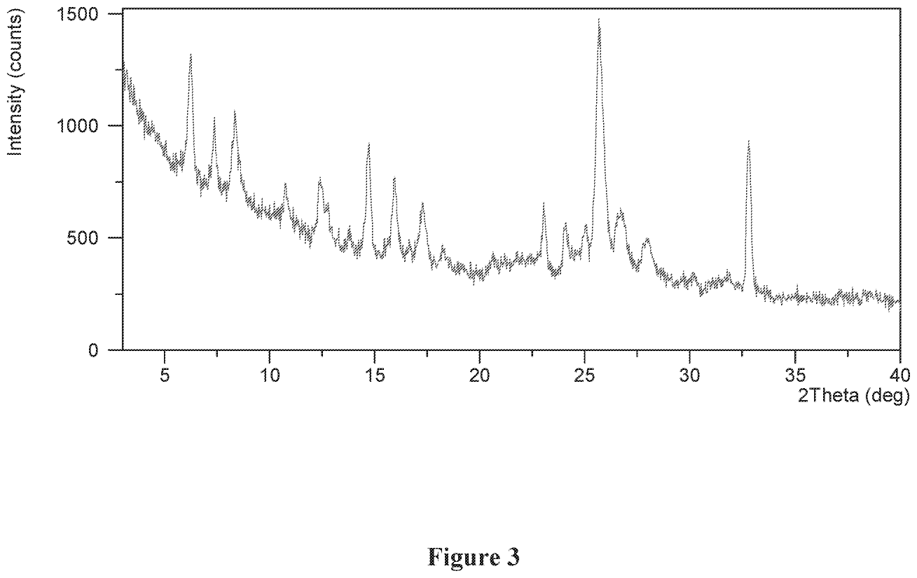

FIG. 4A shows XRPD pattern of Compound 4R freebase Type A.

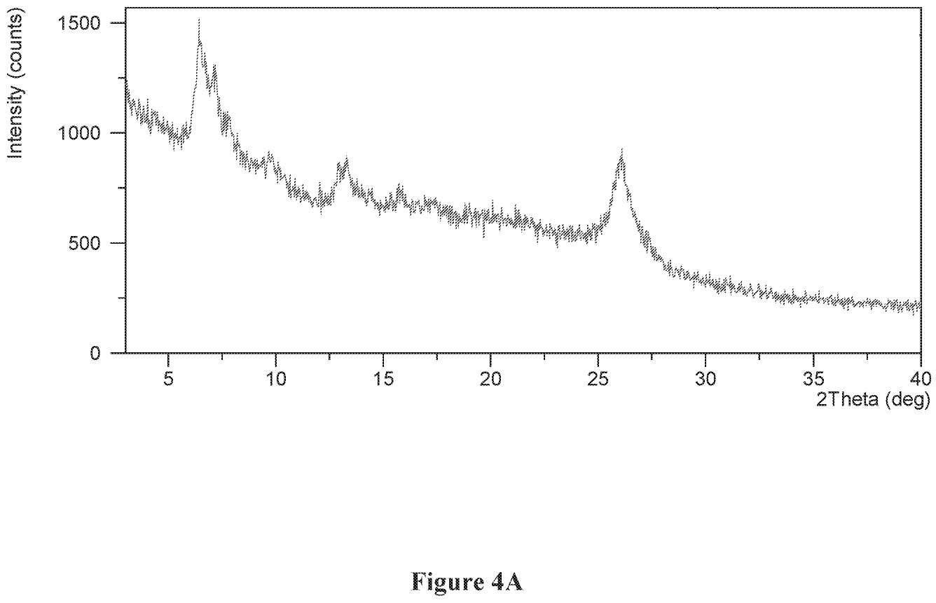

FIG. 4B shows XRPD pattern of Compound 4R freebase Type B.

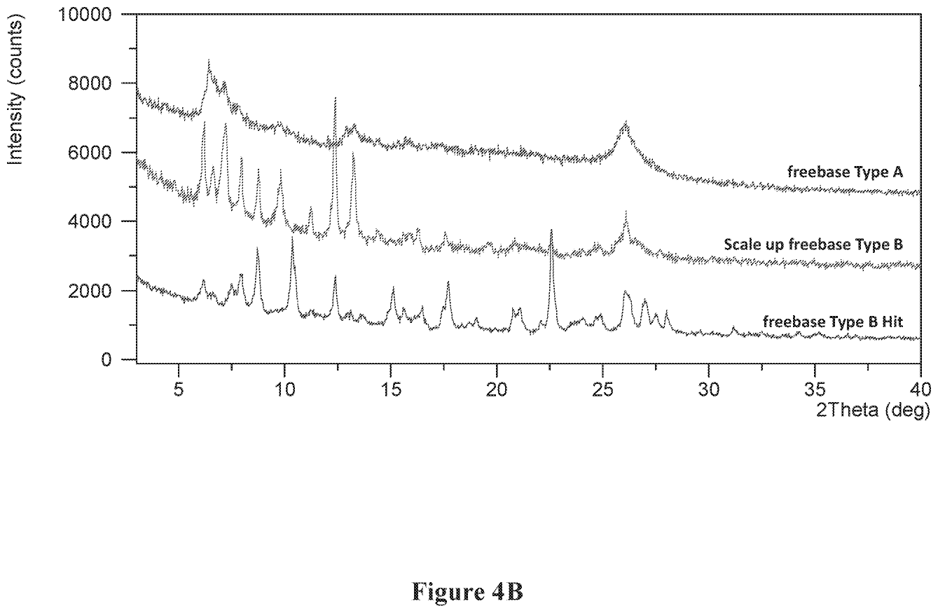

FIG. 5A shows XRPD pattern of Compound 5R freebase Type A.

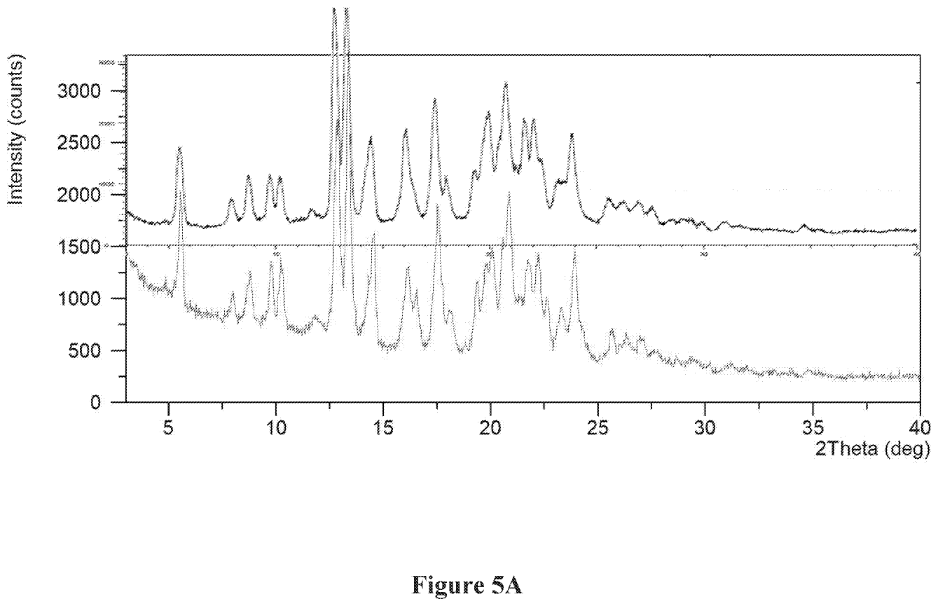

FIG. 5B shows XRPD of Compound 5R freebase Type A, and XRPD of the compound after heating to 130° C. (freebase Type B).

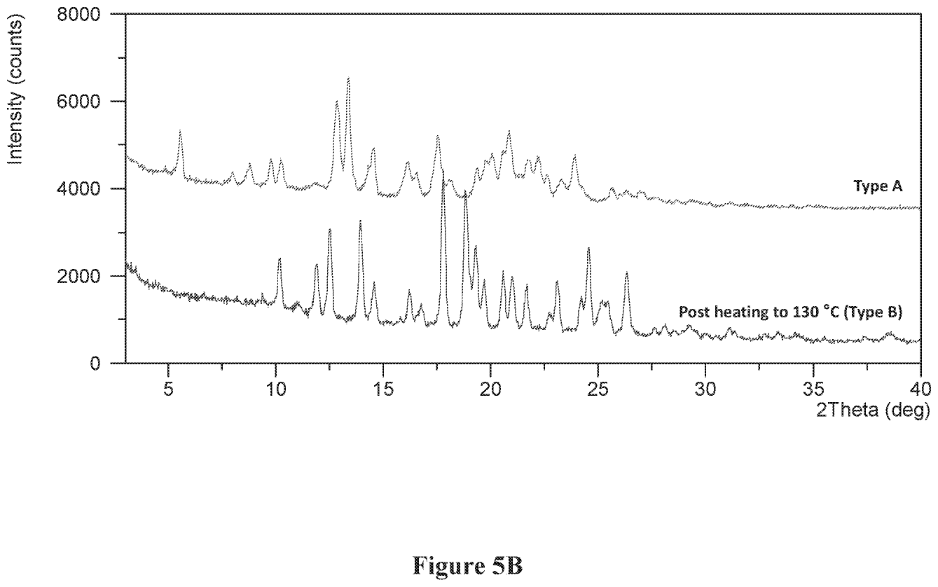

FIG. 5C shows XRPD pattern of Compound 5R freebase Type B.

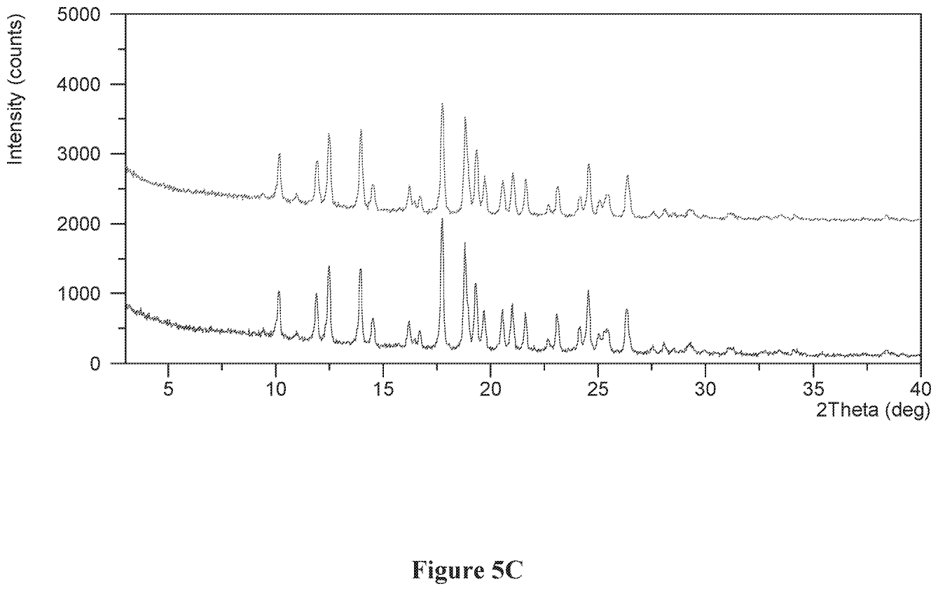

FIG. 5D shows XRPD overlay of Compound 5R freebase Type B before and after DVS.

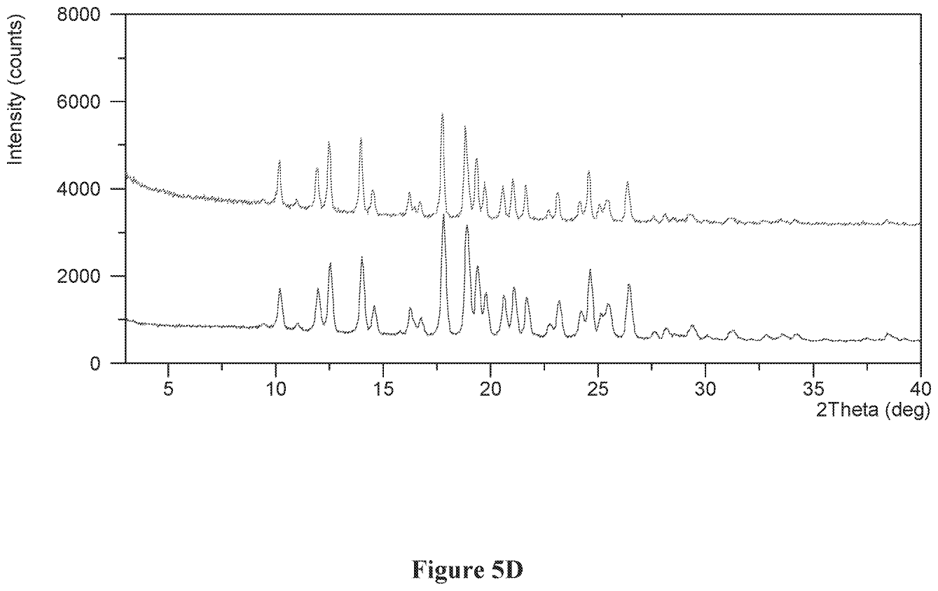

FIG. 6 shows XRPD pattern of Compound 6 freebase Type A.

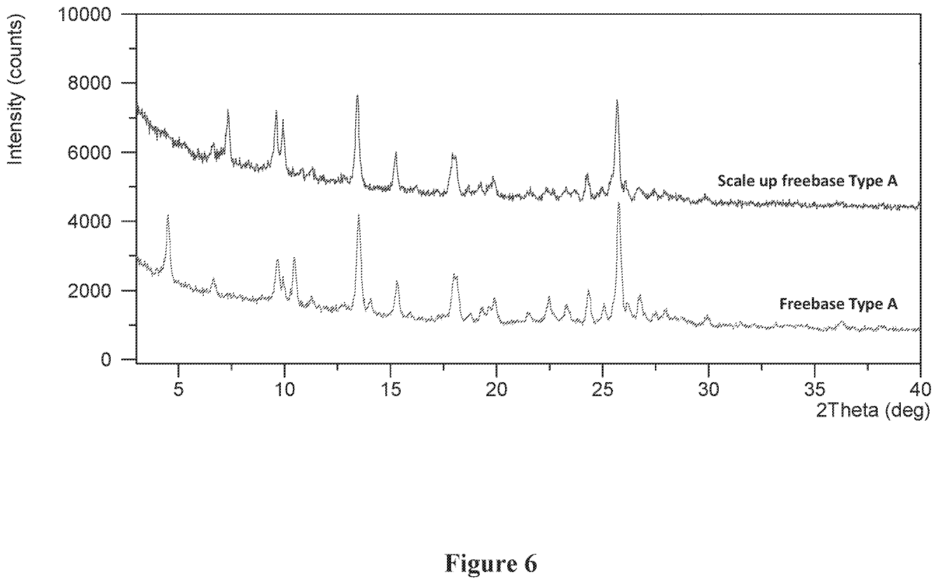

### DETAILED DESCRIPTION

The present disclosure provides novel amine-substituted heterocyclic compounds, synthetic methods for making the compounds, pharmaceutical compositions containing them and various uses of the compounds.

In one aspect, the present disclosure provides a compound selected from the group consisting of

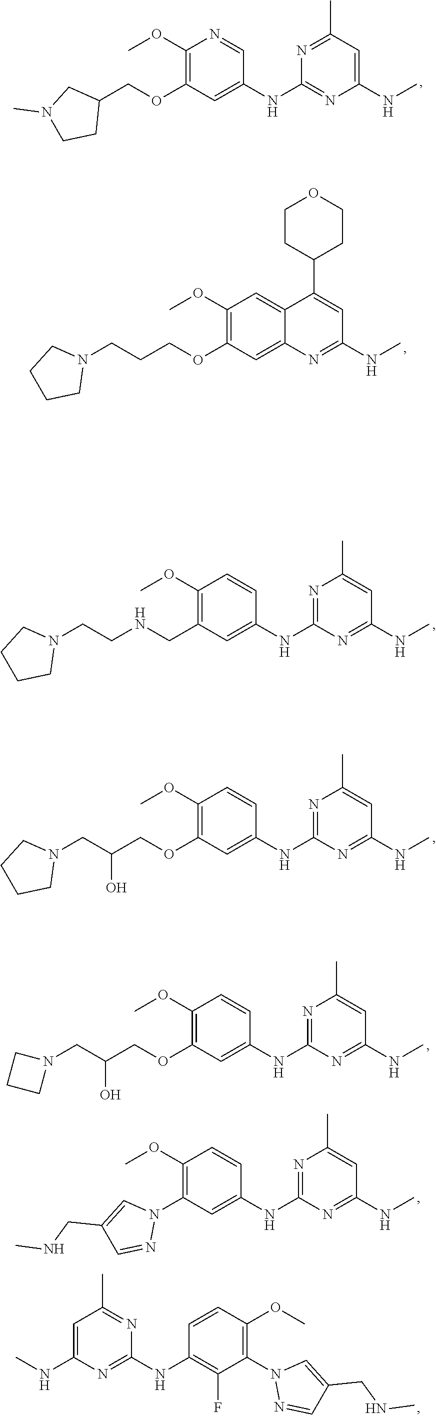

tautomers thereof, pharmaceutically acceptable salts thereof, and pharmaceutically acceptable salts of the tautomers.

In some embodiments, the compound is selected from the compounds listed in Table 1, pharmaceutically acceptable salts thereof, and pharmaceutically acceptable salts of the tautomers.

TABLE 1

| Compound No. | Structure |
|---|---|
| 1 | 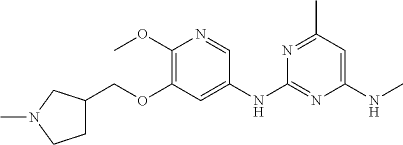 |
| 1R | 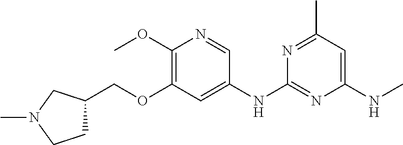 |
| 1S | 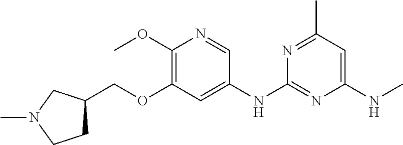 |
| 2 | 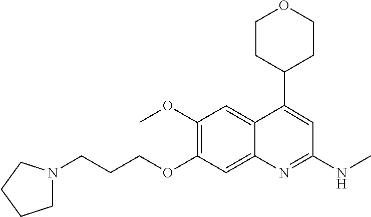 [SMILES: CNc1cc(C2CCOCC2)c2cc(OC)c(OCCCN3CCCC3)cc2n1] |
| 3 | 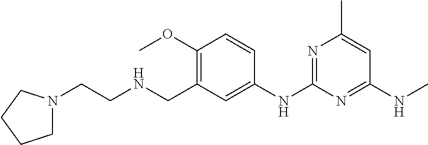 [SMILES: CNc1cc(C)nc(Nc2ccc(OC)c(CNCCN3CCCC3)c2)n1] |
| 4 | 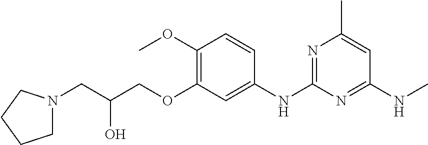 [SMILES: CNc1cc(C)nc(Nc2ccc(OC)c(OCC(O)CN3CCCC3)c2)n1] |
| 4R | 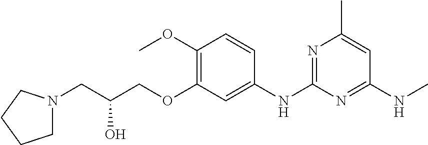 [SMILES: CNc1cc(C)nc(Nc2ccc(OC)c(OC[C@H](O)CN3CCCC3)c2)n1] |
| 4S | 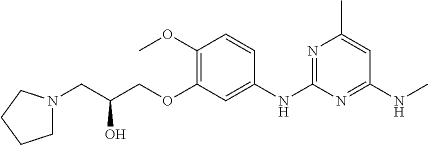 [SMILES: CNc1cc(C)nc(Nc2ccc(OC)c(OC[C@@H](O)CN3CCCC3)c2)n1] |
| 5 | 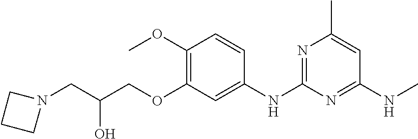 [SMILES: CNc1cc(C)nc(Nc2ccc(OC)c(OCC(O)CN3CCC3)c2)n1] |
| 5R | 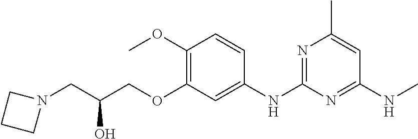 [SMILES: CNc1cc(C)nc(Nc2ccc(OC)c(OC[C@@H](O)CN3CCC3)c2)n1] |
| 5S | 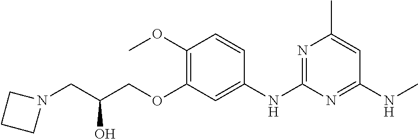 [SMILES: CNc1cc(C)nc(Nc2ccc(OC)c(OC[C@@H](O)CN3CCC3)c2)n1] |
| 6 | 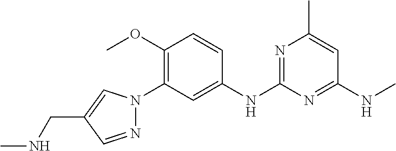 |
| 7 | 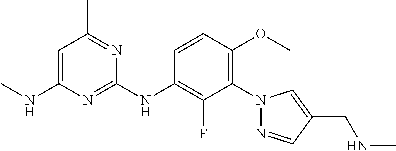 |

In some embodiments, the compound is selected from the compounds listed in Table 1.

In some embodiments, the compound is a crystalline form of any one of the compounds listed in Table 1.

In some embodiments, the compound (e.g., the crystalline form of any one of the compounds listed in Table 1) is an anhydrate (e.g., an anhydrate of any one of the compounds listed in Table 1).

In some embodiments, the compound is selected from pharmaceutically acceptable salts of the compounds listed in Table 1.

In some embodiments, the compound is a crystalline form of any one of the pharmaceutically acceptable salts of the compounds listed in Table 1.

In some embodiments, the compound (e.g., the crystalline form of any one of the pharmaceutically acceptable salts of the compounds listed in Table 1) is an anhydrate (e.g., an anhydrate of any one of the pharmaceutically acceptable salts of the compounds listed in Table 1).

In some embodiments, the compound is selected from hydrochloride salts, sulfate salts, glycolate salts, adipate salts, succinate salts, oxalate salts, phosphate salts, fumarate salts, hippurate salts, gentisate salts, and benzoate salts of the compounds listed in Table 1.

In some embodiments, the compound is selected from hydrochloride salts of the compounds listed in Table 1.

In some embodiments, the compound is a crystalline form of any one of the hydrochloride salts of the compounds listed in Table 1.

In some embodiments, the compound is selected from sulfate salts of the compounds listed in Table 1.

In some embodiments, the compound is a crystalline form of any one of the sulfate salts of the compounds listed in Table 1.

In some embodiments, the compound is selected from glycolate salts of the compounds listed in Table 1.

In some embodiments, the compound is a crystalline form of any one of the glycolate salts of the compounds listed in Table 1.

In some embodiments, the compound is selected from adipate salts of the compounds listed in Table 1.

In some embodiments, the compound is a crystalline form of any one of the adipate salts of the compounds listed in Table 1.

In some embodiments, the compound is selected from succinate salts of the compounds listed in Table 1.

In some embodiments, the compound is a crystalline form of any one of the succinate salts of the compounds listed in Table 1.

In some embodiments, the compound is selected from oxalate salts of the compounds listed in Table 1.

In some embodiments, the compound is a crystalline form of any one of the oxalate salts of the compounds listed in Table 1.

In some embodiments, the compound is selected from phosphate salts of the compounds listed in Table 1.

In some embodiments, the compound is a crystalline form of any one of the phosphate salts of the compounds listed in Table 1.

In some embodiments, the compound is selected from fumarate salts of the compounds listed in Table 1.

In some embodiments, the compound is a crystalline form of any one of the fumarate salts of the compounds listed in Table 1.

In some embodiments, the compound is selected from hippurate salts of the compounds listed in Table 1.

In some embodiments, the compound is a crystalline form of any one of the hippurate salts of the compounds listed in Table 1.

In some embodiments, the compound is selected from gentisate salts of the compounds listed in Table 1.

In some embodiments, the compound is a crystalline form of any one of the gentisate salts of the compounds listed in Table 1.

In some embodiments, the compound is selected from benzoate salts of the compounds listed in Table 1.

In some embodiments, the compound is a crystalline form of any one of the benzoate salts of the compounds listed in Table 1.

### Compound 1

In some embodiments, the compound is

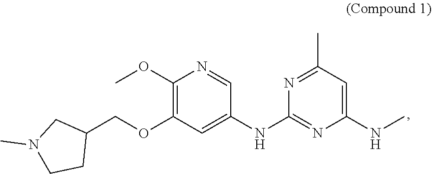

a tautomer thereof, a pharmaceutically acceptable salt thereof, or a pharmaceutically acceptable salt of the tautomer.

In some embodiments, the compound is Compound 1.

In some embodiments, the compound is

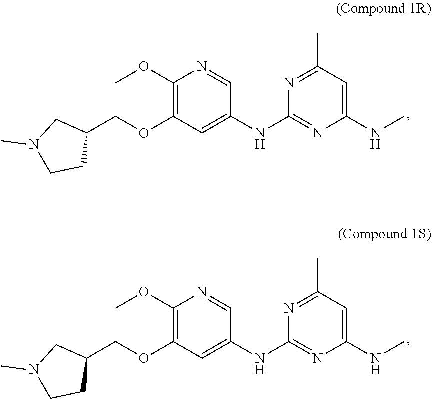

a tautomer thereof, a pharmaceutically acceptable salt thereof, or a pharmaceutically acceptable salt of the tautomer.

In some embodiments, the compound is Compound 1R or Compound 1S.

In some embodiments, the compound is Compound 1R, a tautomer thereof, a pharmaceutically acceptable salt thereof, or a pharmaceutically acceptable salt of the tautomer.

In some embodiments, the compound is Compound 1R.

In some embodiments, the compound is a crystalline form of Compound 1R.

In some embodiments, the crystalline form of Compound 1R is an anhydrate.

In some embodiments, the compound is a pharmaceutically acceptable salt of Compound 1R.

In some embodiments, the compound is a crystalline form of a pharmaceutically acceptable salt of Compound 1R.

In some embodiments, the crystalline form of the pharmaceutically acceptable salt of Compound 1R is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 1R.

### Compound 1R Freebase Type A

In some embodiments, the compound is Compound 1R.

In some embodiments, the compound is a crystalline form of Compound 1R.

In some embodiments, the compound (e.g., the crystalline form of Compound 1R) has an endothermic peak top temperature in differential scanning calorimeter (DSC) analysis at between about 65° C. and about 105° C., between about 70° C. and about 100° C., between about 75° C. and about 95° C., between about 84° C. and about 90° C., or between about 86° C. and about 88° C.

In some embodiments, the compound (e.g., the crystalline form of Compound 1R) has an endothermic peak top temperature in differential scanning calorimeter (DSC) analysis at at between about 180° C. and about 220° C., between about 185° C. and about 215° C., between about 190° C. and about 210° C., between about 195° C. and about 205° C., or between about 198° C. and about 200° C.

In some embodiments, the compound (e.g., the crystalline form of Compound 1R) has an endothermic peak top temperature in differential scanning calorimeter (DSC) analysis at about 86.9° C. and/or at about 199.1° C.

In some embodiments, the compound (e.g., the crystalline form of Compound 1R) has an endothermic peak top temperature in modulated differential scanning calorimeter (mDSC) analysis at between about 190° C. and about 230° C., between about 195° C. and about 225° C., between about 200° C. and about 220° C., between about 204° C. and about 212° C., between about 206° C. and about 210° C., or between about 207° C. and about 209° C.

In some embodiments, compound (e.g., the crystalline form of Compound 1R) has an endothermic peak top temperature in modulated differential scanning calorimeter (mDSC) analysis at about 208° C.

### Compound 1R Freebase Type B

In some embodiments, the compound is Compound 1R.

In some embodiments, the compound is a crystalline form of Compound 1R.

In some embodiments, the compound (e.g., the crystalline form of the hydrochloride salt of Compound 1R) is characterized by an XRPD pattern having at least one peak selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least two peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least three peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least four peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least five peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least six peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having at least seven peaks selected from 6.4±0.2, 11.8±0.2, 14.2±0.2, 18.2110.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having one peak selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having two peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having three peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having four peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having five peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having six peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having seven peaks selected from 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having a peak at 6.4±0.2, 11.8±0.2, 14.2±0.2, 18.21±0.2, 19.2±0.2, 25.7±0.2, 26.4±0.2, and 29.3±0.2 °2θ (e.g., 6.4±0.1, 11.8±0.1, 14.2±0.1, 18.21±0.1, 19.2±0.1, 25.7±0.1, 26.4±0.1, and 29.3±0.1 °2θ) using Cu Kα radiation.

In some embodiments, the compound (e.g., the crystalline form of the hydrochloride salt of Compound 1R) is characterized by an XRPD pattern having a peak at from about 6.2 to about 6.6, from about 25.5 to about 25.9, and from about 26.2 to about 26.6 °2θ using Cu Kα radiation.

In some embodiments, the compound (e.g., the crystalline form of the hydrochloride salt of Compound 1R) is characterized by an XRPD pattern having a peak at from about 6.2 to about 6.6, from about 14.0 to about 14.4, from about 25.5 to about 25.9, and from about 26.2 to about 26.6 °2θ using Cu Kα radiation.

In some embodiments, the compound (e.g., the crystalline form of the hydrochloride salt of Compound 1R) is characterized by an XRPD pattern having a peak at from about 6.2 to about 6.6, from about 14.0 to about 14.4, from about 18.0 to about 18.4, from about 25.5 to about 25.9, and from about 26.2 to about 26.6 °2θ using Cu Kα radiation.

In some embodiments, the compound (e.g., the crystalline form of the hydrochloride salt of Compound 1R) is characterized by an XRPD pattern having a peak at from about 6.2 to about 6.6, from about 14.0 to about 14.4, from about 18.0 to about 18.4, from about 19.0 to about 19.4, from about 25.5 to about 25.9, and from about 26.2 to about 26.6 °2θ using Cu Kα radiation.

In some embodiments, the compound (e.g., the crystalline form of the hydrochloride salt of Compound 1R) is characterized by an XRPD pattern having a peak at from about 6.2 to about 6.6, from about 11.6 to about 12.0, from about 14.0 to about 14.4, from about 18.0 to about 18.4, from about 19.0 to about 19.4, from about 25.5 to about 25.9, and from about 26.2 to about 26.6 °2θ using Cu Kα radiation.

In some embodiments, the compound (e.g., the crystalline form of the hydrochloride salt of Compound 1R) is characterized by an XRPD pattern having a peak at from about 6.2 to about 6.6, from about 11.6 to about 12.0, from about 14.0 to about 14.4, from about 18.0 to about 18.4, from about 19.0 to about 19.4, from about 25.5 to about 25.9, from about 26.2 to about 26.6, and from about 29.1 to about 29.5 °2θ using Cu Kα radiation.

In some embodiments, the compound (e.g., the crystalline form of the hydrochloride salt of Compound 1R) is characterized by an XRPD pattern having a peak at from about 6.3 to about 6.5, from about 11.7 to about 11.9, from about 14.1 to about 14.3, from about 18.1 to about 18.3, from about 19.1 to about 19.3, from about 25.6 to about 25.8, from about 26.3 to about 26.5, and from about 29.2 to about 29.4 °2θ using Cu Kα radiation.

In some embodiments, the compound (e.g., the crystalline form of the hydrochloride salt of Compound 1R) is characterized by an XRPD pattern having a peak at about 6.39, about 11.80, about 14.20, about 18.21, about 19.15, about 25.67, about 26.41, and about 29.31 °2θ using Cu Kα radiation.

In some embodiments, the compound (e.g., the crystalline form of Compound 1R) has an endothermic peak top temperature in differential scanning calorimeter (DSC) analysis at between about 200° C. and about 240° C., between about 205° C. and about 235° C., between about 210° C. and about 230° C., between about 215° C. and about 227° C., between about 219° C. and about 225° C., or between about 221° C. and about 223° C.

In some embodiments, the compound (e.g., the crystalline form of Compound 1R) has an endothermic peak top temperature in differential scanning calorimeter (DSC) analysis at about 222.1° C.

### Compound 1R Hydrochloride Salt Type A

In some embodiments, the compound is a hydrochloride salt of Compound 1R.

In some embodiments, the compound is a crystalline form of a hydrochloride salt of Compound 1R.

In some embodiments, the compound (e.g., the crystalline form of the hydrochloride salt of Compound 1R) is characterized by an XRPD pattern having at least one peak selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least two peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least three peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least four peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least five peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least six peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having at least seven peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having one peak selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having two peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having three peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having four peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having five peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having six peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having seven peaks selected from 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having a peak at 6.2±0.2, 7.2±0.2, 8.0±0.2, 8.8±0.2, 12.4±0.2, 13.3±0.2, 17.7±0.2, and 26.2±0.2 °2θ (e.g., 6.2±0.1, 7.2±0.1, 8.0±0.1, 8.8±0.1, 12.4±0.1, 13.3±0.1, 17.7±0.1, and 26.2±0.1 °2θ) using Cu Kα radiation.

In some embodiments, the compound (e.g., the crystalline form of the hydrochloride salt of Compound 1R) is characterized by an XRPD pattern having a peak at from about 7.8 to about 8.2 and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 7.8 to about 8.2, from about 8.6 to about 9.0, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 7.8 to about 8.2, from about 8.6 to about 9.0, from from about 12.2 to about 12.6, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 6.0 to about 6.4, from about 7.8 to about 8.2, from about 8.6 to about 9.0, from from about 12.2 to about 12.6, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 6.0 to about 6.4, from about 7.0 to about 7.4, from about 7.8 to about 8.2, from about 8.6 to about 9.0, from from about 12.2 to about 12.6, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 6.0 to about 6.4, from about 7.0 to about 7.4, from about 7.8 to about 8.2, from about 8.6 to about 9.0, from from about 12.2 to about 12.6, from about 13.0 to about 13.4, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 6.0 to about 6.4, from about 7.0 to about 7.4, from about 7.8 to about 8.2, from about 8.6 to about 9.0, from from about 12.2 to about 12.6, from about 13.0 to about 13.4, from about 17.4 to about 17.8, and from about 26.0 to about 26.4 °2θ using Cu Kα radiation.

In some embodiments, the compound (e.g., the crystalline form of the hydrochloride salt of Compound 1R) is characterized by an XRPD pattern having a peak at from about 6.1 to about 6.3, from about 7.1 to about 7.3, from about 7.9 to about 8.1, from about 8.7 to about 8.9, from from about 12.3 to about 12.5, from about 13.1 to about 13.3, from about 17.5 to about 17.7, and from about 26.1 to about 26.3 °2θ using Cu Kα radiation.

In some embodiments, the compound (e.g., the crystalline form of the hydrochloride salt of Compound 1R) is characterized by an XRPD pattern having a peak at about 6.19, about 7.22, about 8.00, about 8.83, about 12.42, about 13.26, about 17.65, and about 26.20 °2θ using Cu Kα radiation.

In some embodiments, the compound (e.g., the crystalline form of the hydrochloride salt of Compound 1R) has an endothermic peak top temperature in differential scanning calorimeter (DSC) analysis at between about 50° C. and about 90° C., between about 60° C. and about 80° C., between about 65° C. and about 78° C., between about 70° C. and about 75° C., or between about 72° C. and about 74° C.

In some embodiments, the compound (e.g., the crystalline form of the hydrochloride salt of Compound 1R) has an endothermic peak top temperature in differential scanning calorimeter (DSC) analysis at between about 230° C. and about 270° C., between about 235° C. and about 265° C., between about 240° C. and about 260° C., between about 245° C. and about 255° C., or between about 249° C. and about 251° C.

In some embodiments, the compound (e.g., the crystalline form of the hydrochloride salt of Compound 1R) has an endothermic peak top temperature in differential scanning calorimeter (DSC) analysis at about 72.7° C. and/or at about 249.6° C.

In some embodiments, the compound is Compound 1 S, a tautomer thereof, a pharmaceutically acceptable salt thereof, or a pharmaceutically acceptable salt of the tautomer.

In some embodiments, the compound is Compound 1S.

In some embodiments, the compound is a crystalline form of Compound 1S.

In some embodiments, the compound is a pharmaceutically acceptable salt of Compound 1S.

In some embodiments, the compound is a crystalline form of a pharmaceutically acceptable salt of Compound 1S.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 1S.

### Compound 2

In some embodiments, the compound is (Compound

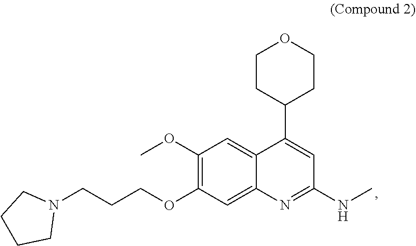 [SMILES: CNc1cc(C2CCOCC2)c2cc(OC)c(OCCCN3CCCC3)cc2n1]

a tautomer thereof, a pharmaceutically acceptable salt thereof, or a pharmaceutically acceptable salt of the tautomer.

In some embodiments, the compound is Compound 2.

In some embodiments, the compound is a crystalline form of Compound 2.

In some embodiments, the crystalline form of Compound 2 is an anhydrate.

In some embodiments, the compound is a pharmaceutically acceptable salt of Compound 2.

In some embodiments, the compound is a crystalline form of a pharmaceutically acceptable salt of Compound 2.

In some embodiments, the crystalline form of the pharmaceutically acceptable salt of Compound 2 is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 2.

### Compound 2 Freebase Type A

In some embodiments, the compound is Compound 2.

In some embodiments, the compound is a crystalline form of Compound 2.

In some embodiments, the compound (e.g., the crystalline form of Compound 2) is characterized by an XRPD pattern having at least one peak selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least two peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least three peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least four peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least five peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least six peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having at least seven peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having one peak selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having two peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having three peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having four peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having five peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having six peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and a 30.0±0.1 °2θ) using Cu Kα radiation.

In some embodiments, the compound (e.g., the crystalline form of Compound 2) is characterized by an XRPD pattern having seven peaks selected from 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having a peak at 8.0±0.2, 9.6±0.2, 12.6±0.2, 15.7±0.2, 16.0±0.2, 18.6±0.2, 19.2±0.2, 19.6±0.2, 23.2±0.2, and 30.0±0.2 °2θ (e.g., 8.0±0.1, 9.6±0.1, 12.6±0.1, 15.7±0.1, 16.0±0.1, 18.6±0.1, 19.2±0.1, 19.6±0.1, 23.2±0.1, and 30.0±0.1 °2θ) using Cu Kα radiation.

In some embodiments, the compound (e.g., the crystalline form of Compound 2) is characterized by an XRPD pattern having a peak at from about 7.8 to about 8.2, from about 12.4 to about 12.8, and from about 19.4 to about 19.8 °2θ using Cu Kα radiation.

In some embodiments, the compound (e.g., the crystalline form of Compound 2) is characterized by an XRPD pattern having a peak at from about 7.8 to about 8.2, from about 12.4 to about 12.8, from about 15.5 to about 15.9, and from about 19.4 to about 19.8 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, and from about 19.4 to about 19.8 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, from about 19.0 to about 19.4, and from about 19.4 to about 19.8 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, from about 19.0 to about 19.4, from about 19.4 to about 19.8, and from about 29.8 to about 30.2 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, from about 19.0 to about 19.4, from about 19.4 to about 19.8, from about 23.0 to about 23.4, and from about 29.8 to about 30.2 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, from about 15.8 to about 16.2, from about 19.0 to about 19.4, from about 19.4 to about 19.8, from about 23.0 to about 23.4, and from about 29.8 to about 30.2 °2θ using Cu Kα radiation.

In some embodiments, the compound (e.g., the crystalline form of Compound 2) is characterized by an XRPD pattern having a peak at from about 7.8 to about 8.2, from about 9.4 to about 9.8, from about 12.4 to about 12.8, from about 15.5 to about 15.9, from about 15.8 to about 16.2, from about 18.4 to about 18.8, from about 19.0 to about 19.4, from about 19.4 to about 19.8, from about 23.0 to about 23.4, and from about 29.8 to about 30.2 °2θ using Cu Kα radiation.

In some embodiments, the compound (e.g., the crystalline form of Compound 2) is characterized by an XRPD pattern having a peak at from about 7.9 to about 8.1, from about 9.5 to about 9.7, from about 12.5 to about 12.7, from about 15.6 to about 15.8, from about 15.9 to about 16.1, from about 18.5 to about 18.7, from about 19.1 to about 19.3, from about 19.5 to about 19.7, from about 23.1 to about 23.3, and from about 29.9 to about 30.1 °2θ using Cu Kα radiation.

In some embodiments, the compound (e.g., the crystalline form of Compound 2) is characterized by an XRPD pattern having a peak at about 7.98, about 9.56, about 12.59, about 15.68, about 15.97, about 18.62, about 19.18, about 19.57, about 23.19, and about 30.04 °2θ using Cu Kα radiation.

In some embodiments, the compound (e.g., the crystalline form of Compound 2) has an endothermic peak top temperature in differential scanning calorimeter (DSC) analysis at between about 160° C. and about 200° C., between about 165° C. and about 195° C., between about 170° C. and about 190° C., between about 175° C. and about 185° C., between about 177° C. and about 183° C., or between about 179° C. and about 181° C.

In some embodiments, the compound (e.g., the crystalline form of Compound 2) has an endothermic peak top temperature in differential scanning calorimeter (DSC) analysis at about 180.4° C.

### Compound 2 Hydrochloride Salt Type A

In some embodiments, the compound is a hydrochloride salt of Compound 2.

In some embodiments, the compound is a crystalline form of a hydrochloride salt of Compound 2.

In some embodiments, the compound (e.g., the crystalline form of the hydrochloride salt of Compound 2) is characterized by an XRPD pattern having at least one peak selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least two peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least three peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least four peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least five peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least six peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having at least seven peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having one peak selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having two peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having three peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having four peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having five peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having six peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having seven peaks selected from 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having a peak at 5.3±0.2, 8.3±0.2, 9.9±0.2, 16.7±0.2, 17.5±0.2, 20.3±0.2, 25.1±0.2, and 27.0±0.2 °2θ (e.g., 5.3±0.1, 8.3±0.1, 9.9±0.1, 16.7±0.1, 17.5±0.1, 20.3±0.1, 25.1±0.1, and 27.0±0.1 °2θ) using Cu Kα radiation.

In some embodiments, the compound (e.g., the crystalline form of the hydrochloride salt of Compound 2) is characterized by an XRPD pattern having a peak at from about 5.1 to about 5.4 and from about 17.3 to about 17.7 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 9.7 to about 10.1, and from about 17.3 to about 17.7 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 9.7 to about 10.1, from about 17.3 to about 17.7, and from about about 20.1 to about 20.5 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 8.1 to about 8.5, from about 9.7 to about 10.1, from about 17.3 to about 17.7, and from about about 20.1 to about 20.5 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 8.1 to about 8.5, from about 9.7 to about 10.1, from about 16.5 to about 16.9, from about 17.3 to about 17.7, and from about about 20.1 to about 20.5 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 8.1 to about 8.5, from about 9.7 to about 10.1, from about 16.5 to about 16.9, from about 17.3 to about 17.7, from about about 20.1 to about 20.5, and from about 26.8 to about 27.2 °2θ using Cu Kα radiation.

In some embodiments, the compound (e.g., the crystalline form of the hydrochloride salt of Compound 2) is characterized by an XRPD pattern having a peak at from about 5.1 to about 5.4, from about 8.1 to about 8.5, from about 9.7 to about 10.1, from about 16.5 to about 16.9, from about 17.3 to about 17.7, from about about 20.1 to about 20.5, from about 24.9 to about 25.3, and from about 26.8 to about 27.2 °2θ using Cu Kα radiation.

In some embodiments, the compound (e.g., the crystalline form of the hydrochloride salt of Compound 2) is characterized by an XRPD pattern having a peak at from about 5.2 to about 5.3, from about 8.2 to about 8.4, from about 9.8 to about 10.0, from about 16.6 to about 16.8, from about 17.4 to about 17.6, from about about 20.2 to about 20.4, from about 25.0 to about 25.2, and from about 26.9 to about 27.1 °2θ using Cu Kα radiation.

In some embodiments, the compound (e.g., the crystalline form of the hydrochloride salt of Compound 2) is characterized by an XRPD pattern having a peak at about 5.29, about 8.32, about 9.87, about 16.67, about 17.51, about 20.30, about 25.10, and about 27.04 °2θ using Cu Kα radiation.

### Compound 3

In some embodiments, the compound is

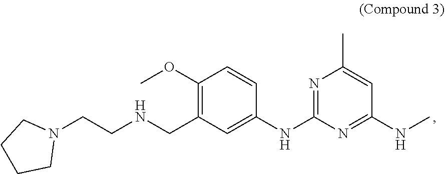 [SMILES: CNc1cc(C)nc(Nc2ccc(OC)c(CNCCN3CCCC3)c2)n1]

a tautomer thereof, a pharmaceutically acceptable salt thereof, or a pharmaceutically acceptable salt of the tautomer.

In some embodiments, the compound is Compound 3.

In some embodiments, the compound is a crystalline form of Compound 3.

In some embodiments, the crystalline form of Compound 3 is an anhydrate.

In some embodiments, the compound is a pharmaceutically acceptable salt of Compound 3.

In some embodiments, the compound is a crystalline form of a pharmaceutically acceptable salt of Compound 3.

In some embodiments, the crystalline form of the pharmaceutically acceptable salt of Compound 3 is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 3.

### Compound 3 Freebase Type A

In some embodiments, the compound is Compound 3.

In some embodiments, the compound is a crystalline form of Compound 3.

In some embodiments, the compound (e.g., the crystalline form of Compound 3) is characterized by an XRPD pattern having at least one peak selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least two peaks selected from 6.3±0.2, 8.3±0.2, 12.4±0.2, 14.7±0.2 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least three peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least four peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least five peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least six peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least seven peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having at least eight peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having one peak selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having two peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having three peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having four peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having five peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having six peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having seven peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having eight peaks selected from 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having a peak at 6.3±0.2, 8.3±0.2, 12.4±0.2, 14.7±0.2, 15.9±0.2, 17.3±0.2, 23.1±0.2, 25.6±0.2, and 32.7±0.2 °2θ (e.g., 6.3±0.1, 8.3±0.1, 12.4±0.1, 14.7±0.1, 15.9±0.1, 17.3±0.1, 23.1±0.1, 25.6±0.1, and 32.7±0.1 °2θ) using Cu Kα radiation.

In some embodiments, the compound (e.g., the crystalline form of Compound 3) is characterized by an XRPD pattern having a peak at from about 6.2 to about 6.4, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 14.6 to about 14.8, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 8.33 to about 8.35, from about 14.6 to about 14.8, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 8.33 to about 8.35, from about 14.6 to about 14.8, from about 15.8 to about 16.0, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 8.33 to about 8.35, from about 12.3 to about 12.5, from about 14.6 to about 14.8, from about 15.8 to about 16.0, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 8.33 to about 8.35, from about 12.3 to about 12.5, from about 14.6 to about 14.8, from about 15.8 to about 16.0, from about 23.0 to about 23.2, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at from about 6.2 to about 6.4, from about 8.33 to about 8.35, from about 12.3 to about 12.5, from about 14.6 to about 14.8, from about 15.8 to about 16.0, from about 17.2 to about 17.4, from about 23.0 to about 23.2, from about 25.5 to about 25.7, and from about 32.6 to about 32.8 °2θ using Cu Kα radiation.

In some embodiments, the compound (e.g., the crystalline form of Compound 3) is characterized by an XRPD pattern having a peak at about 6.27, about 8.34, about 12.41, about 14.73, about 15.94, about 17.28, about 23.07, about 25.64, and about 32.74 °2θ using Cu Kα radiation.

In some embodiments, the compound (e.g., the crystalline form of Compound 3) has an endothermic peak top temperature in differential scanning calorimeter (DSC) analysis at between about 70° C. and about 110° C., between about 75° C. and about 105° C., between about 80° C. and about 100° C., between about 90° C. and about 96° C., or between about 92° C. and about 94° C.

In some embodiments, the compound (e.g., the crystalline form of Compound 3) has an endothermic peak top temperature in differential scanning calorimeter (DSC) analysis at between about 130° C. and about 170° C., between about 135° C. and about 165° C., between about 140° C. and about 160° C., between about 148° C. and about 155° C., or between about 150° C. and about 153° C.

In some embodiments, the compound (e.g., the crystalline form of Compound 3) has an endothermic peak top temperature in differential scanning calorimeter (DSC) analysis at about 93.2° C. and/or at about 151.6° C.

### Compound 3 Hydrochloride Salt Type A

In some embodiments, the compound is a hydrochloride salt of Compound 3.

In some embodiments, the compound is a crystalline form of a hydrochloride salt of Compound 3.

In some embodiments, the compound (e.g., the crystalline form of the hydrochloride salt of Compound 3) is characterized by an XRPD pattern having at least one peak selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least two peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least three peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least four peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least five peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least six peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having at least seven peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having one peak selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having two peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having three peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having four peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having five peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having six peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having seven peaks selected from 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having a peak at 6.8±0.2, 9.0±0.2, 11.8±0.2, 16.3±0.2, 25.1±0.2, 25.6±0.2, 26.3±0.2, and 27.6±0.2 °2θ (e.g., 6.8±0.1, 9.0±0.1, 11.8±0.1, 16.3±0.1, 25.1±0.1, 25.6±0.1, 26.3±0.1, and 27.6±0.1 °2θ) using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, and from about 25.4 to about 25.8 °2θ using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, from about 25.4 to about 25.8, and from about 27.4 to about 27.8 °2θ using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, from about 24.9 to about 25.3, from about 25.4 to about 25.8, and from about 27.4 to about 27.8 °2θ using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, from about 24.9 to about 25.3, from about 25.4 to about 25.8, from about 26.1 to about 26.5, and from about 27.4 to about 27.8 °2θ using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, from about 16.1 to about 16.5, from about 24.9 to about 25.3, from about 25.4 to about 25.8, from about 26.1 to about 26.5, and from about 27.4 to about 27.8 °2θ using Cu Kα radiation.

In some embodiments, the compound (e.g., the crystalline form of the hydrochloride salt of Compound 3) is characterized by an XRPD pattern having a peak at from about 6.6 to about 7.0, from about 8.8 to about 9.2, from about 11.6 to about 12.0, from about 16.1 to about 16.5, from about 24.9 to about 25.3, from about 25.4 to about 25.8, from about 26.1 to about 26.5, and from about 27.4 to about 27.8 °2θ using Cu Kα radiation.

In some embodiments, the compound (e.g., the crystalline form of the hydrochloride salt of Compound 3) is characterized by an XRPD pattern having a peak at from about 6.7 to about 6.9, from about 8.9 to about 9.1, from about 11.7 to about 11.9, from about 16.2 to about 16.4, from about 25.0 to about 25.2, from about 25.5 to about 25.7, from about 26.2 to about 26.4, and from about 27.5 to about 27.7 °2θ using Cu Kα radiation.

In some embodiments, the compound (e.g., the crystalline form of the hydrochloride salt of Compound 3) is characterized by an XRPD pattern having a peak at about 6.82, about 9.00, about 11.80, about 16.30, about 25.05, about 25.56, about 26.33, and about 27.61 °2θ using Cu Kα radiation.

In some embodiments, the compound (e.g., the crystalline form of the hydrochloride salt of Compound 3) has an endothermic peak top temperature in differential scanning calorimeter (DSC) analysis at between about 75° C. and about 115° C., between about 80° C. and about 100° C., between about 85° C. and about 105° C., between about 90° C. and about 100° C., or between about 95° C. and about 96° C.

In some embodiments, the compound (e.g., the crystalline form of the hydrochloride salt of Compound 3) has an endothermic peak top temperature in differential scanning calorimeter (DSC) analysis at about 95.5° C.

### Compound 3 Hydrochloride Salt Type B

In some embodiments, the compound is a hydrochloride salt of Compound 3.

In some embodiments, the compound is a crystalline form of a hydrochloride salt of Compound 3.

In some embodiments, the compound (e.g., the crystalline form of the hydrochloride salt of Compound 3) is characterized by an XRPD pattern having at least one peak selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least two peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least three peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least four peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least five peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least six peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least seven peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least eight peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having at least nine peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having one peak selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having two peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having three peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having four peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having five peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having six peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having seven peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having eight peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having nine peaks selected from 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having a peak at 11.8±0.2, 12.3±0.2, 16.9±0.2, 22.3±0.2, 23.1±0.2, 23.6±0.2, 25.3±0.2, 27.5±0.2, 28.1±0.2, and 30.1±0.2 °2θ (e.g., 11.8±0.1, 12.3±0.1, 16.9±0.1, 22.3±0.1, 23.1±0.1, 23.6±0.1, 25.3±0.1, 27.5±0.1, 28.1±0.1, and 30.1±0.1 °2θ) using Cu Kα radiation.

In some embodiments, the compound (e.g., the crystalline form of the hydrochloride salt of Compound 3) is characterized by an XRPD pattern having a peak at from about 12.1 to about 12.5, from about 16.7 to about 17.0, and from about 25.1 to about 25.5 °2θ using Cu Kα radiation.

In some embodiments, the compound (e.g., the crystalline form of the hydrochloride salt of Compound 3) is characterized by an XRPD pattern having a peak at from about 12.1 to about 12.5, from about 16.7 to about 17.0, from about 25.1 to about 25.5, and from about 27.3 to about 27.7 °2θ using Cu Kα radiation.

In some embodiments, the compound (e.g., the crystalline form of the hydrochloride salt of Compound 3) is characterized by an XRPD pattern having a peak at from about 12.1 to about 12.5, from about 16.7 to about 17.0, from about 22.1 to about 22.5, from about 25.1 to about 25.5, and from about 27.3 to about 27.7 °2θ using Cu Kα radiation.

In some embodiments, the compound (e.g., the crystalline form of the hydrochloride salt of Compound 3) is characterized by an XRPD pattern having a peak at from 11.5 to about 11.9, from about 12.1 to about 12.5, from about 16.7 to about 17.0, from about 22.1 to about 22.5, from about 25.1 to about 25.5, and from about 27.3 to about 27.7 °2θ using Cu Kα radiation.

In some embodiments, the compound (e.g., the crystalline form of the hydrochloride salt of Compound 3) is characterized by an XRPD pattern having a peak at from 11.5 to about 11.9, from about 12.1 to about 12.5, from about 16.7 to about 17.0, from about 22.1 to about 22.5, from about 25.1 to about 25.5, from about 27.3 to about 27.7, and from about 29.8 to about 30.2 °2θ using Cu Kα radiation.

In some embodiments, the compound (e.g., the crystalline form of the hydrochloride salt of Compound 3) is characterized by an XRPD pattern having a peak at from 11.5 to about 11.9, from about 12.1 to about 12.5, from about 16.7 to about 17.0, from about 22.1 to about 22.5, from about 23.4 to about 23.8, from about 25.1 to about 25.5, from about 27.3 to about 27.7, and from about 29.8 to about 30.2 °2θ using Cu Kα radiation.

In some embodiments, the compound (e.g., the crystalline form of the hydrochloride salt of Compound 3) is characterized by an XRPD pattern having a peak at from 11.5 to about 11.9, from about 12.1 to about 12.5, from about 16.7 to about 17.0, from about 22.1 to about 22.5, from about 23.4 to about 23.8, from about 25.1 to about 25.5, from about 27.3 to about 27.7, from about 27.9 to about 28.3, and from about 29.8 to about 30.2 °2θ using Cu Kα radiation.

In some embodiments, the compound (e.g., the crystalline form of the hydrochloride salt of Compound 3) is characterized by an XRPD pattern having a peak at from 11.5 to about 11.9, from about 12.1 to about 12.5, from about 16.7 to about 17.0, from about 22.1 to about 22.5, from about 22.9 to about 23.3, from about 23.4 to about 23.8, from about 25.1 to about 25.5, from about 27.3 to about 27.7, from about 27.9 to about 28.3, and from about 29.8 to about 30.2 °2θ using Cu Kα radiation.

In some embodiments, the compound (e.g., the crystalline form of the hydrochloride salt of Compound 3) is characterized by an XRPD pattern having a peak at from 11.6 to about 11.8, from about 12.2 to about 12.4, from about 16.8 to about 16.9, from about 22.2 to about 22.4, from about 23.0 to about 23.2, from about 23.5 to about 23.7, from about 25.2 to about 25.4, from about 27.4 to about 27.6, from about 28.0 to about 28.2, and from about 29.9 to about 30.1 °2θ using Cu Kα radiation.

In some embodiments, the compound (e.g., the crystalline form of the hydrochloride salt of Compound 3) is characterized by an XRPD pattern having a peak at about 11.75, about 12.30, about 16.85, about 22.33, about 23.06, about 23.57, about 25.33, about 27.50, about 28.05, and about 30.06 °2θ using Cu Kα radiation.

In some embodiments, the compound (e.g., the crystalline form of the hydrochloride salt of Compound 3) has an endothermic peak top temperature in differential scanning calorimeter (DSC) analysis at between about 235° C. and about 275° C., between about 240° C. and about 270° C., between about 245° C. and about 265° C., between about 250° C. and about 260° C., or between about 255° C. and about 257° C.

In some embodiments, the compound (e.g., the crystalline form of the hydrochloride salt of Compound 3) has an endothermic peak top temperature in differential scanning calorimeter (DSC) analysis at about 256° C.

### Compound 3 Sulfate Salt Type A

In some embodiments, the compound is a sulfate salt of Compound 3.

In some embodiments, the compound is a crystalline form of a sulfate salt of Compound 3.

In some embodiments, the compound (e.g., the crystalline form of the sulfate salt of Compound 3) is characterized by an XRPD pattern having at least one peak selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least two peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least three peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least four peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least five peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least six peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least seven peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least eight peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having at least nine peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having one peak selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having two peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having three peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having four peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having five peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having six peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having seven peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having eight peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having nine peaks selected from 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having a peak at 5.2±0.2, 10.9±0.2, 14.6±0.2, 18.3±0.2, 19.6±0.2, 20.9±0.2, 22.5±0.2, 24.2±0.2, 25.6±0.2, and 28.0±0.2 °2θ (e.g., 5.2±0.1, 10.9±0.1, 14.6±0.1, 18.3±0.1, 19.6±0.1, 20.9±0.1, 22.5±0.1, 24.2±0.1, 25.6±0.1, and 28.0±0.1 °2θ) using Cu Kα radiation.

In some embodiments, the compound (e.g., the crystalline form of the sulfate salt of Compound 3) is characterized by an XRPD pattern having a peak at from about 5.0 to about 5.4, from about 14.4 to about 14.8, and from about 25.4 to about 25.8 °2θ using Cu Kα radiation.

In some embodiments, the compound (e.g., the crystalline form of the sulfate salt of Compound 3) is characterized by an XRPD pattern having a peak at from about 5.0 to about 5.4, from about 14.4 to about 14.8, from about 20.7 to about 21.0, and from about 25.4 to about 25.8 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 20.7 to about 21.0, and from about 25.4 to about 25.8 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 18.1 to about 18.4, from about 20.7 to about 21.0, and from about 25.4 to about 25.8 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 18.1 to about 18.4, from about 20.7 to about 21.0, from about 25.4 to about 25.8, and from about 27.8 to about 28.2 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 18.1 to about 18.4, from about 19.4 to about 19.8, from about 20.7 to about 21.0, from about 25.4 to about 25.8, and from about 27.8 to about 28.2 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 18.1 to about 18.4, from about 19.4 to about 19.8, from about 20.7 to about 21.0, from about 24.0 to about 24.4, from about 25.4 to about 25.8, and from about 27.8 to about 28.2 °2θ using Cu Kα radiation.

In some embodiments, the compound (e.g., the crystalline form of the sulfate salt of Compound 3) is characterized by an XRPD pattern having a peak at from about 5.0 to about 5.4, from about 10.7 to about 11.0, from about 14.4 to about 14.8, from about 18.1 to about 18.4, from about 19.4 to about 19.8, from about 20.7 to about 21.0, from about 22.3 to about 22.7, from about 24.0 to about 24.4, from about 25.4 to about 25.8, and from about 27.8 to about 28.2 °2θ using Cu Kα radiation.

In some embodiments, the compound (e.g., the crystalline form of the sulfate salt of Compound 3) is characterized by an XRPD pattern having a peak at from about 5.1 to about 5.3, from about 10.8 to about 10.9, from about 14.5 to about 14.7, from about 18.2 to about 18.3, from about 19.5 to about 19.7, from about 20.8 to about 20.9, from about 22.4 to about 22.6, from about 24.1 to about 24.3, from about 25.5 to about 25.7, and from about 27.9 to about 28.1 °2θ using Cu Kα radiation.

In some embodiments, the compound (e.g., the crystalline form of the sulfate salt of Compound 3) is characterized by an XRPD pattern having a peak at about 5.22, about 10.85, about 14.60, about 18.25, about 19.63, about 20.88, about 22.52, about 24.24, about 25.58, and about 27.97 °2θ using Cu Kα radiation.

In some embodiments, the compound (e.g., the crystalline form of the sulfate salt of Compound 3) has an endothermic peak top temperature in differential scanning calorimeter (DSC) analysis at between about 150° C. and about 190° C., between about 155° C. and about 185° C., between about 160° C. and about 180° C., between about 165° C. and about 175° C., or between about 170° C. and about 172° C.;

In some embodiments, the compound (e.g., the crystalline form of the sulfate salt of Compound 3) has an endothermic peak top temperature in differential scanning calorimeter (DSC) analysis at between about 200° C. and about 235° C., between about 205° C. and about 230° C., between about 210° C. and about 225° C., between about 215° C. and about 220° C., or between about 217° C. and about 218° C.;

In some embodiments, the compound (e.g., the crystalline form of the sulfate salt of Compound 3) has an endothermic peak top temperature in differential scanning calorimeter (DSC) analysis at between about 205° C. and about 245° C., between about 210° C. and about 240° C., between about 215° C. and about 235° C., between about 220° C. and about 230° C., or between about 225° C. and about 227° C.

In some embodiments, the compound (e.g., the crystalline form of the sulfate salt of Compound 3) has an endothermic peak top temperature in differential scanning calorimeter (DSC) analysis at between about 255° C. and about 295° C., between about 260° C. and about 290° C., between about 265° C. and about 285° C., between about 270° C. and about 280° C., or between about 275° C. and about 276° C.

In some embodiments, the compound (e.g., the crystalline form of the sulfate salt of Compound 3) has an endothermic peak top temperature in differential scanning calorimeter (DSC) analysis at about 170.9° C., at about 217.3° C., at about 226.4° C., and/or at about 275.3° C.

### Compound 3 Glycolate Salt Type A

In some embodiments, the compound is a glycolate salt of Compound 3.

In some embodiments, the compound is a crystalline form of a glycolate salt of Compound 3.

In some embodiments, the compound (e.g., the crystalline form of the glycolate salt of Compound 3) is characterized by an XRPD pattern having at least one peak selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least two peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least three peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least four peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least five peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least six peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least seven peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least eight peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having at least nine peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having one peak selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having two peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having three peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having four peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having five peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having six peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having seven peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having eight peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having nine peaks selected from 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having a peak at 6.8±0.2, 9.0±0.2, 11.8±0.2, 13.2±0.2, 16.3±0.2, 20.4±0.2, 23.6±0.2, 25.0±0.2, 25.5±0.2, and 27.6±0.2 °2θ (e.g., 6.8±0.1, 9.0±0.1, 11.8±0.1, 13.2±0.1, 16.3±0.1, 20.4±0.1, 23.6±0.1, 25.0±0.1, 25.5±0.1, and 27.6±0.1 °2θ) using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, and from about 24.8 to about 25.2 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, and from about 24.8 to about 25.2 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 24.8 to about 25.2, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 16.1 to about 16.5, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 16.1 to about 16.5, from about 23.4 to about 23.8, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 12.9 to about 13.3, from about 16.1 to about 16.5, from about 23.4 to about 23.8, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 12.9 to about 13.3, from about 16.1 to about 16.5, from about 20.2 to about 20.6, from about 23.4 to about 23.8, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.6 to about 7.0, from about 8.8 to about 9.2, from about 11.6 to about 12.0, from about 12.9 to about 13.3, from about 16.1 to about 16.5, from about 20.2 to about 20.6, from about 23.4 to about 23.8, from about 24.8 to about 25.2, from about 25.3 to about 25.7, and from about 27.4 to about 27.8 °2θ using Cu Kα radiation.

In some embodiments, the compound (e.g., the crystalline form of the glycolate salt of Compound 3) is characterized by an XRPD pattern having a peak at from about 6.7 to about 6.9, from about 8.9 to about 9.1, from about 11.7 to about 11.9, from about 13.0 to about 13.2, from about 16.2 to about 16.4, from about 20.3 to about 20.5, from about 23.5 to about 23.7, from about 24.9 to about 25.1, from about 25.4 to about 25.6, and from about 27.5 to about 27.7 °2θ using Cu Kα radiation.

In some embodiments, the compound (e.g., the crystalline form of the glycolate salt of Compound 3) is characterized by an XRPD pattern having a peak at about 6.81, about 9.00, about 11.77, about 13.15, about 16.28, about 20.44, about 23.63, about 25.02, about 25.52, and about 27.59 °2θ using Cu Kα radiation.

In some embodiments, the compound (e.g., the crystalline form of the glycolate salt of Compound 3) has an endothermic peak top temperature in differential scanning calorimeter (DSC) analysis at between about 80° C. and about 115° C., between about 85° C. and about 110° C., between about 90° C. and about 105° C., between about 95° C. and about 100° C., or between about 97° C. and about 98° C.

In some embodiments, the compound (e.g., the crystalline form of the glycolate salt of Compound 3) has an endothermic peak top temperature in differential scanning calorimeter (DSC) analysis at between about 90° C. and about 130° C., between about 95° C. and about 125° C., between about 100° C. and about 120° C., between about 105° C. and about 115° C., or between about 111° C. and about 112° C.

In some embodiments, the compound (e.g., the crystalline form of the glycolate salt of Compound 3) has an endothermic peak top temperature in differential scanning calorimeter (DSC) analysis at between about 165° C. and about 205° C., between about 170° C. and about 200° C., between about 175° C. and about 195° C., between about 180° C. and about 190° C., or between about 184° C. and about 185° C.

In some embodiments, the compound (e.g., the crystalline form of the glycolate salt of Compound 3) has an endothermic peak top temperature in differential scanning calorimeter (DSC) analysis at between about 235° C. and about 275° C., between about 240° C. and about 270° C., between about 245° C. and about 265° C., between about 250° C. and about 260° C., or between about 254° C. and about 255° C.

In some embodiments, the compound (e.g., the crystalline form of the glycolate salt of Compound 3) has an endothermic peak top temperature in differential scanning calorimeter (DSC) analysis at about 97.4° C., at about 111.5° C., at about 184.7° C., and/or at about 254.4° C.

In some embodiments, the compound (e.g., the crystalline form of the glycolate salt of Compound 3) has an endothermic peak top temperature in differential scanning calorimeter (DSC) analysis at between about 85° C. and about 125° C., between about 90° C. and about 120° C., between about 95° C. and about 115° C., between about 100° C. and about 110° C., or between about 103° C. and about 105° C.

In some embodiments, the compound (e.g., the crystalline form of the glycolate salt of Compound 3) has an endothermic peak top temperature in differential scanning calorimeter (DSC) analysis at between about 115° C. and about 150° C., between about 120° C. and about 145° C., between about 125° C. and about 140° C., between about 130° C. and about 135° C., or between about 132° C. and about 133° C.

In some embodiments, the compound (e.g., the crystalline form of the glycolate salt of Compound 3) has an endothermic peak top temperature in differential scanning calorimeter (DSC) analysis at between about 210° C. and about 250° C., between about 215° C. and about 245° C., between about 220° C. and about 240° C., between about 225° C. and about 235° C., or between about 231° C. and about 233° C.

In some embodiments, the compound (e.g., the crystalline form of the glycolate salt of Compound 3) has an endothermic peak top temperature in differential scanning calorimeter (DSC) analysis at about 103.9° C., at about 132.6° C., and/or at about 231.9° C.

### Compound 3 Succinate Salt Type A

In some embodiments, the compound is a succinate salt of Compound 3.

In some embodiments, the compound is a crystalline form of a succinate salt of Compound 3.

In some embodiments, the compound (e.g., the crystalline form of the succinate salt of Compound 3) is characterized by an XRPD pattern having at least one peak selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least two peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least three peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least four peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least five peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least six peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least seven peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least eight peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having at least nine peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having one peak selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having two peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having three peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having four peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having five peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having six peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having seven peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having eight peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having nine peaks selected from 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having a peak at 6.8±0.2, 7.6±0.2, 9.0±0.2, 11.8±0.2, 14.8±0.2, 22.1±0.2, 23.3±0.2, 25.7±0.2, 27.3±0.2, and 32.7±0.2 °2θ (e.g., 6.8±0.1, 7.6±0.1, 9.0±0.1, 11.8±0.1, 14.8±0.1, 22.1±0.1, 23.3±0.1, 25.7±0.1, 27.3±0.1, and 32.7±0.1 °2θ) using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, and from about 25.5 to about 25.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 25.5 to about 25.9, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 11.6 to about 12.0, from about 25.5 to about 25.9, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 11.6 to about 12.0, from about 23.1 to about 23.5, from about 25.5 to about 25.9, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 8.8 to about 9.2, from about 11.6 to about 12.0, from about 23.1 to about 23.5, from about 25.5 to about 25.9, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 8.8 to about 9.2, from about 11.6 to about 12.0, from about 23.1 to about 23.5, from about 25.5 to about 25.9, from about 27.1 to about 27.5, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 8.8 to about 9.2, from about 11.6 to about 12.0, from about 14.6 to about 15.0, from about 23.1 to about 23.5, from about 25.5 to about 25.9, from about 27.1 to about 27.5, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.6 to about 7.0, from about 7.4 to about 7.8, from about 8.8 to about 9.2, from about 11.6 to about 12.0, from about 14.6 to about 15.0, from about 21.9 to about 22.2, from about 23.1 to about 23.5, from about 25.5 to about 25.9, from about 27.1 to about 27.5, and from about 32.5 to about 32.9 °2θ using Cu Kα radiation.

In some embodiments, the compound (e.g., the crystalline form of the succinate salt of Compound 3) is characterized by an XRPD pattern having a peak at from about 6.7 to about 6.9, from about 7.5 to about 7.7, from about 8.9 to about 9.1, from about 11.7 to about 11.9, from about 14.7 to about 14.9, from about 22.0 to about 22.1, from about 23.2 to about 23.4, from about 25.6 to about 25.8, from about 27.2 to about 27.4, and from about 32.6 to about 32.8 °2θ using Cu Kα radiation.

In some embodiments, the compound (e.g., the crystalline form of the succinate salt of Compound 3) is characterized by an XRPD pattern having a peak at about 6.84, about 7.56, about 8.98, about 11.77, about 14.79, about 22.05, about 23.31, about 25.69, about 27.32, and about 32.74 °2θ using Cu Kα radiation.

In some embodiments, the compound (e.g., the crystalline form of the succinate salt of Compound 3) has an endothermic peak top temperature in differential scanning calorimeter (DSC) analysis at between about 75° C. and about 110° C., between about 80° C. and about 105° C., between about 85° C. and about 100° C., between about 90° C. and about 95° C., or between about 92° C. and about 93° C.

In some embodiments, the compound (e.g., the crystalline form of the succinate salt of Compound 3) has an endothermic peak top temperature in differential scanning calorimeter (DSC) analysis at between about 165° C. and about 200° C., between about 170° C. and about 195° C., between about 175° C. and about 190° C., between about 180° C. and about 185° C., or between about 182° C. and about 183° C.

In some embodiments, the compound (e.g., the crystalline form of the succinate salt of Compound 3) has an endothermic peak top temperature in differential scanning calorimeter (DSC) analysis at about 92.4° C. and/or at about 182.2° C.

### Compound 4

In some embodiments, the compound is

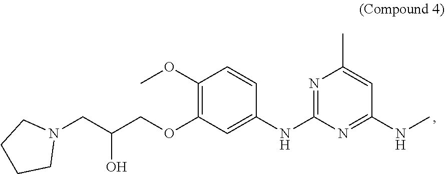 [SMILES: CNc1cc(C)nc(Nc2ccc(OC)c(OCC(O)CN3CCCC3)c2)n1]

a tautomer thereof, a pharmaceutically acceptable salt thereof, or a pharmaceutically acceptable salt of the tautomer.

In some embodiments, the compound is Compound 4.

In some embodiments, the compound is

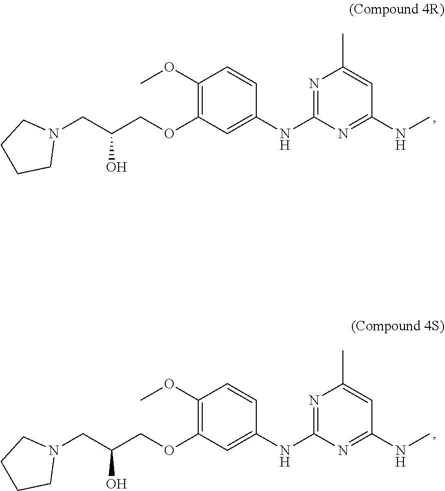 [SMILES: CNc1cc(C)nc(Nc2ccc(OC)c(OC[C@H](O)CN3CCCC3)c2)n1 ; CNc1cc(C)nc(Nc2ccc(OC)c(OC[C@@H](O)CN3CCCC3)c2)n1]

a tautomer thereof, a pharmaceutically acceptable salt thereof, or a pharmaceutically acceptable salt of the tautomer.

In some embodiments, the compound is Compound 4R or Compound 4S.

In some embodiments, the compound is Compound 4R, a tautomer thereof, a pharmaceutically acceptable salt thereof, or a pharmaceutically acceptable salt of the tautomer.

In some embodiments, the compound is 4R.

In some embodiments, the compound is a crystalline form of Compound 4R.

In some embodiments, the crystalline form of Compound 4R is an anhydrate.

In some embodiments, the compound is a pharmaceutically acceptable salt of Compound 4R.

In some embodiments, the compound is a crystalline form of a pharmaceutically acceptable salt of Compound 4R.

In some embodiments, the crystalline form of the pharmaceutically acceptable salt of Compound 4R is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 4R.

### Compound 4R Freebase Type A

In some embodiments, the compound is 4R.

In some embodiments, the compound is a crystalline form of Compound 4R.

In some embodiments, the compound (e.g., the crystalline form of Compound 4R) is characterized by an XRPD pattern having at least one peak selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having at least two peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having at least three peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having at least four peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having at least five peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having one peak selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having two peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having three peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having four peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having five peaks selected from 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having a peak at 6.4±0.2, 7.2±0.2, 9.9±0.2, 13.3±0.2, 15.7±0.2, and 26.1±0.2 °2θ (e.g., 6.4±0.1, 7.2±0.1, 9.9±0.1, 13.3±0.1, 15.7±0.1, and 26.1±0.1 °2θ) using Cu Kα radiation.

In some embodiments, the compound (e.g., the crystalline form of Compound 4R) is characterized by an XRPD pattern having a peak at from about 6.2 to about 6.6, from about 7.0 to about 7.4, and from about 25.9 to about 26.3 °2θ using Cu Kα radiation.

In some embodiments, the compound (e.g., the crystalline form of Compound 4R) is characterized by an XRPD pattern having a peak at from about 6.2 to about 6.6, from about 7.0 to about 7.4, from about 13.1 to about 13.5, and from about 25.9 to about 26.3 °2θ using Cu Kα radiation.

In some embodiments, the compound (e.g., the crystalline form of Compound 4R) is characterized by an XRPD pattern having a peak at from about 6.2 to about 6.6, from about 7.0 to about 7.4, from about 13.1 to about 13.5, from about 15.5 to about 15.9, and from about 25.9 to about 26.3 °2θ using Cu Kα radiation.

In some embodiments, the compound (e.g., the crystalline form of Compound 4R) is characterized by an XRPD pattern having a peak at from about 6.2 to about 6.6, from about 7.0 to about 7.4, from about 9.7 to about 10.1, from about 13.1 to about 13.5, from about 15.5 to about 15.9, and from about 25.9 to about 26.3 °2θ using Cu Kα radiation.

In some embodiments, the compound (e.g., the crystalline form of Compound 4R) is characterized by an XRPD pattern having a peak at from about 6.3 to about 6.5, from about 7.1 to about 7.3, from about 9.8 to about 10.0, from about 13.2 to about 13.4, from about 15.6 to about 15.8, and from about 26.0 to about 26.2 °2θ using Cu Kα radiation.

In some embodiments, the compound (e.g., the crystalline form of Compound 4R) is characterized by an XRPD pattern having a peak at about 6.40, about 7.17, about 9.86, about 13.31, about 15.71, and about 26.10 °2θ using Cu Kα radiation.

In some embodiments, the compound (e.g., the crystalline form of Compound 4R) has an endothermic peak top temperature in differential scanning calorimeter (DSC) analysis at between about 60° C. and about 100° C., between about 65° C. and about 95° C., between about 70° C. and about 90° C., between about 74° C. and about 82° C., or between about 77° C. and about 79° C.

In some embodiments, the compound (e.g., the crystalline form of Compound 4R) has an endothermic peak top temperature in differential scanning calorimeter (DSC) analysis at between about 210° C. and about 250° C., between about 215° C. and about 245° C., between about 220° C. and about 240° C., between about 225° C. and about 233° C., or between about 228° C. and about 230° C.

In some embodiments, the compound (e.g., the crystalline form of Compound 4R) has an endothermic peak top temperature in differential scanning calorimeter (DSC) analysis at about 77.8° C. and/or at about 229.2° C.

In some embodiments, the compound (e.g., the crystalline form of Compound 4R) has an endothermic peak top temperature in modulated differential scanning calorimeter (mDSC) analysis at between about 200° C. and about 240° C., between about 205° C. and about 235° C., between about 210° C. and about 230° C., between about 215° C. and about 225° C., or between about 218° C. and about 220° C.

In some embodiments, the compound (e.g., the crystalline form of Compound 4R) has an endothermic peak top temperature in modulated differential scanning calorimeter (mDSC) analysis at about 219.2° C.

### Compound 4R Freebase Type B

In some embodiments, the compound is 4R.

In some embodiments, the compound is a crystalline form of Compound 4R.

In some embodiments, the compound (e.g., the crystalline form of Compound 4R) is characterized by an XRPD pattern having at least one peak selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least two peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least three peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least four peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least five peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least six peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least seven peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least eight peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having at least nine peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having one peak selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having two peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having three peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having four peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having five peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having six peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having seven peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having eight peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having nine peaks selected from 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having a peak at 6.3±0.2, 6.7±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.7±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of Compound 4R) is characterized by an XRPD pattern having a peak at from about 6.1 to about 6.5, from about 6.5 to about 6.9, and from about 9.0 to about 9.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, and from about 12.9 to about 13.3 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.9 to about 13.3, and from about 26.0 to about 26.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.9 to about 13.3, from about 19.9 to about 20.3, and from about 26.0 to about 26.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.5 to about 12.9, from about 12.9 to about 13.3, from about 19.9 to about 20.3, and from about 26.0 to about 26.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.5 to about 12.9, from about 12.9 to about 13.3, from about 14.2 to about 14.6, from about 19.9 to about 20.3, and from about 26.0 to about 26.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.5 to about 12.9, from about 12.9 to about 13.3, from about 14.2 to about 14.6, from about 19.9 to about 20.3, from about 21.8 to about 22.2, and from about 26.0 to about 26.4 °2θ using Cu Kα radiation.

In some embodiments, the compound (e.g., the crystalline form of Compound 4R) is characterized by an XRPD pattern having a peak at from about 6.1 to about 6.5, from about 6.5 to about 6.9, from about 9.0 to about 9.4, from about 12.5 to about 12.9, from about 12.9 to about 13.3, from about 14.2 to about 14.6, from about 19.9 to about 20.3, from about 21.8 to about 22.2, from about 26.0 to about 26.4, and from about 26.9 to about 27.3 °2θ using Cu Kα radiation.

In some embodiments, the compound (e.g., the crystalline form of Compound 4R) is characterized by an XRPD pattern having a peak at from about 6.2 to about 6.4, from about 6.6 to about 6.8, from about 9.1 to about 9.3, from about 12.6 to about 12.8, from about 13.0 to about 13.2, from about 14.3 to about 14.5, from about 20.0 to about 20.2, from about 21.9 to about 22.1, from about 26.1 to about 26.3, and from about 27.0 to about 27.2 °2θ using Cu Kα radiation.

In some embodiments, the compound (e.g., the crystalline form of Compound 4R) is characterized by an XRPD pattern having a peak at about 6.31, about 6.73, about 9.24, about 12.66, about 13.13, about 14.37, about 20.08, about 22.0, about 26.15, and about 27.05 °2θ using Cu Kα radiation.

In some embodiments, the compound (e.g., the crystalline form of Compound 4R) has an endothermic peak top temperature in differential scanning calorimeter (DSC) analysis at between about 60° C. and about 100° C., between about 65° C. and about 95° C., between about 70° C. and about 90° C., between about 74° C. and about 82° C., or between about 77° C. and about 79° C.

In some embodiments, the compound (e.g., the crystalline form of Compound 4R) has an endothermic peak top temperature in differential scanning calorimeter (DSC) analysis at between about 185° C. and about 225° C., between about 190° C. and about 220° C., between about 195° C. and about 215° C., between about 200° C. and about 210° C., or between about 203° C. and about 206° C.

In some embodiments, the compound (e.g., the crystalline form of Compound 4R) has an endothermic peak top temperature in differential scanning calorimeter (DSC) analysis at about 77.5° C. and/or at about 204.6° C.

### Compound 4R Freebase Type C

In some embodiments, the compound is 4R.

In some embodiments, the compound is a crystalline form of Compound 4R.

In some embodiments, the compound (e.g., the crystalline form of Compound 4R) is characterized by an XRPD pattern having at least one peak selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having at least two peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having at least three peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having at least four peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having at least five peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having at least six peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having one peak selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having two peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having three peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having four peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having five peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having six peaks selected from 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having a peak at 7.3±0.2, 8.0±0.2, 8.8±0.2, 9.8±0.2, 12.4±0.2, 13.3±0.2, and 26.2±0.2 °2θ (e.g., 7.3±0.1, 8.0±0.1, 8.8±0.1, 9.8±0.1, 12.4±0.1, 13.3±0.1, and 26.2±0.1 °2θ) using Cu Kα radiation.

In some embodiments, the compound (e.g., the crystalline form of Compound 4R) is characterized by an XRPD pattern having a peak at from about 7.1 to about 7.5, from about about 13.1 to about 13.5, and from about 26.0 to about 26.4 °2θ using Cu Kα radiation.

In some embodiments, the compound (e.g., the crystalline form of Compound 4R) is characterized by an XRPD pattern having a peak at from about 7.1 to about 7.5, from about 9.6 to about 10.0, from about about 13.1 to about 13.5, and from about 26.0 to about 26.4 °2θ using Cu Kα radiation.

In some embodiments, the compound (e.g., the crystalline form of Compound 4R) is characterized by an XRPD pattern having a peak at from about 7.1 to about 7.5, from about 7.8 to about 8.2, from about 9.6 to about 10.0, from about about 13.1 to about 13.5, and from about 26.0 to about 26.4 °2θ using Cu Kα radiation.

In some embodiments, the compound (e.g., the crystalline form of Compound 4R) is characterized by an XRPD pattern having a peak at from about 7.1 to about 7.5, from about 7.8 to about 8.2, from about 9.6 to about 10.0, from about 12.2 to about 12.6, from about about 13.1 to about 13.5, and from about 26.0 to about 26.4 °2θ using Cu Kα radiation.

In some embodiments, the compound (e.g., the crystalline form of Compound 4R) is characterized by an XRPD pattern having a peak at from about 7.1 to about 7.5, from about 7.8 to about 8.2, from about 8.7 to about 9.1, from about 9.6 to about 10.0, from about 12.2 to about 12.6, from about about 13.1 to about 13.5, and from about 26.0 to about 26.4 °2θ using Cu Kα radiation.

In some embodiments, the compound (e.g., the crystalline form of Compound 4R) is characterized by an XRPD pattern having a peak at from about 7.2 to about 7.4, from about 7.9 to about 8.1, from about 8.8 to about 9.0, from about 9.7 to about 9.9, from about 12.3 to about 12.5, from about about 13.2 to about 13.4, and from about 26.1 to about 26.3 °2θ using Cu Kα radiation.

In some embodiments, the compound (e.g., the crystalline form of Compound 4R) is characterized by an XRPD pattern having a peak at about 7.26, about 7.96, about 8.80, about 9.82, about 12.40, about 13.31, and about 26.18 °2θ using Cu Kao radiation.

In some embodiments, the compound (e.g., the crystalline form of Compound 4R) has an endothermic peak top temperature in differential scanning calorimeter (DSC) analysis at between about 230° C. and about 270° C., between about 235° C. and about 265° C., between about 240° C. and about 260° C., between about 245° C. and about 255° C., or between about 247° C. and about 249° C.

In some embodiments, the compound (e.g., the crystalline form of Compound 4R) has an endothermic peak top temperature in differential scanning calorimeter (DSC) analysis at about 248.0° C.

### Compound 4R Hydrochloride Salt Type A

In some embodiments, the compound is a hydrochloride salt of Compound 4R.

In some embodiments, the compound is a crystalline form of a hydrochloride salt of Compound 4R.

In some embodiments, the compound (e.g., the crystalline form of the hydrochloride salt of Compound 4R) is characterized by an XRPD pattern having at least one peak selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least two peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least three peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least four peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least five peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least six peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having at least seven peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having one peak selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having two peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having three peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having four peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having five peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having six peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having seven peaks selected from 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having a peak at 6.3±0.2, 11.8±0.2, 14.5±0.2, 15.5±0.2, 19.4±0.2, 25.5±0.2, 26.3±0.2, and 29.4±0.2 °2θ (e.g., 6.3±0.1, 11.8±0.1, 14.5±0.1, 15.5±0.1, 19.4±0.1, 25.5±0.1, 26.3±0.1, and 29.4±0.1 °2θ) using Cu Kα radiation.

In some embodiments, the compound (e.g., the crystalline form of the hydrochloride salt of Compound 4R) is characterized by an XRPD pattern having a peak at from about 6.1 to about 6.5, from about 14.3 to about 14.7, and from about 25.3 to about 25.7 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 14.3 to about 14.7, and from about 25.3 to about 25.7 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 14.3 to about 14.7, from about 25.3 to about 25.7, and from about 26.1 to about 26.5 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 11.6 to about 12.0, from about 14.3 to about 14.7, from about 25.3 to about 25.7, and from about 26.1 to about 26.5 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 11.6 to about 12.0, from about 14.3 to about 14.7, from about 15.3 to about 15.7, from about 25.3 to about 25.7, and from about 26.1 to about 26.5 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 11.6 to about 12.0, from about 14.3 to about 14.7, from about 15.3 to about 15.7, from about 25.3 to about 25.7, from about 26.1 to about 26.5, and from about 29.2 to about 29.6 °2θ using Cu Kα radiation.

In some embodiments, the compound (e.g., the crystalline form of the hydrochloride salt of Compound 4R) is characterized by an XRPD pattern having a peak at from about 6.1 to about 6.5, from about 11.6 to about 12.0, from about 14.3 to about 14.7, from about 15.3 to about 15.7, from about 19.2 to about 19.6, from about 25.3 to about 25.7, from about 26.1 to about 26.5, and from about 29.2 to about 29.6 °2θ using Cu Kα radiation.

In some embodiments, the compound (e.g., the crystalline form of the hydrochloride salt of Compound 4R) is characterized by an XRPD pattern having a peak at from about 6.2 to about 6.4, from about 11.7 to about 11.9, from about 14.4 to about 14.6, from about 15.4 to about 15.6, from about 19.3 to about 19.5, from about 25.4 to about 25.6, from about 26.2 to about 26.4, and from about 29.3 to about 29.5 °2θ using Cu Kα radiation.

In some embodiments, the compound (e.g., the crystalline form of the hydrochloride salt of Compound 4R) is characterized by an XRPD pattern having a peak at about 6.34, about 11.80, about 14.50, about 15.51, about 19.36, about 25.50, about 26.28, and about 29.38 °2θ using Cu Kα radiation.

In some embodiments, the compound (e.g., the crystalline form of the hydrochloride salt of Compound 4R) has an endothermic peak top temperature in differential scanning calorimeter (DSC) analysis at between about 55° C. and about 95° C., between about 60° C. and about 90° C., between about 65° C. and about 85° C., between about 70° C. and about 80° C., or between about 75° C. and about 76° C.

In some embodiments, the compound (e.g., the crystalline form of the hydrochloride salt of Compound 4R) has an endothermic peak top temperature in differential scanning calorimeter (DSC) analysis at between about 180° C. and about 220° C., between about 185° C. and about 215° C., between about 190° C. and about 210° C., between about 195° C. and about 205° C., or between about 198° C. and about 199° C.

In some embodiments, the compound (e.g., the crystalline form of the hydrochloride salt of Compound 4R) has an endothermic peak top temperature in differential scanning calorimeter (DSC) analysis at about 75.7° C. and/or at about 198.7° C.

### Compound 4R Hydrochloride Salt Type B

In some embodiments, the compound is a hydrochloride salt of Compound 4R.

In some embodiments, the compound is a crystalline form of a hydrochloride salt of Compound 4R.

In some embodiments, the compound (e.g., the crystalline form of the hydrochloride salt of Compound 4R) is characterized by an XRPD pattern having at least one peak selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least two peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least three peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least four peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least five peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least six peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least seven peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having at least eight peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having one peak selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having two peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having three peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having four peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having five peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having six peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having seven peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having eight peaks selected from 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having a peak at 7.2±0.2, 8.0±0.2, 8.8±0.2, 9.8±0.2, 12.4±0.2, 13.3±0.2, 14.4±0.2, 17.6±0.2, and 26.2±0.2 °2θ (e.g., 7.2±0.1, 8.0±0.1, 8.8±0.1, 9.8±0.1, 12.4±0.1, 13.3±0.1, 14.4±0.1, 17.6±0.1, and 26.2±0.1 °2θ) using Cu Kα radiation.

In some embodiments, the compound (e.g., the crystalline form of the hydrochloride salt of Compound 4R) is characterized by an XRPD pattern having a peak at from about 7.0 to about 7.4, from about 12.2 to about 12.6, and from about 13.1 to about 13.5 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 9.6 to about 10.0, from about 12.2 to about 12.6, and from about 13.1 to about 13.5 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 7.8 to about 8.2, from about 9.6 to about 10.0, from about 12.2 to about 12.6, and from about 13.1 to about 13.5 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 7.8 to about 8.2, from about 8.6 to about 9.0, from about 9.6 to about 10.0, from about 12.2 to about 12.6, and from about 13.1 to about 13.5 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 7.8 to about 8.2, from about 8.6 to about 9.0, from about 9.6 to about 10.0, from about 12.2 to about 12.6, from about 13.1 to about 13.5, and from about 26.0 to about 26.4 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 7.8 to about 8.2, from about 8.6 to about 9.0, from about 9.6 to about 10.0, from about 12.2 to about 12.6, from about 13.1 to about 13.5, from about 17.4 to about 17.8, and from about 26.0 to about 26.4 °2θ using Cu Kα radiation.

In some embodiments, the compound (e.g., the crystalline form of the hydrochloride salt of Compound 4R) is characterized by an XRPD pattern having a peak at from about 7.0 to about 7.4, from about 7.8 to about 8.2, from about 8.6 to about 9.0, from about 9.6 to about 10.0, from about 12.2 to about 12.6, from about 13.1 to about 13.5, about 14.2 to about 14.6, from about 17.4 to about 17.8, and from about 26.0 to about 26.4 °2θ using Cu Kα radiation.

In some embodiments, the compound (e.g., the crystalline form of the hydrochloride salt of Compound 4R) is characterized by an XRPD pattern having a peak at from about 7.1 to about 7.3, from about 7.9 to about 8.1, from about 8.7 to about 8.9, from about 9.7 to about 9.9, from about 12.3 to about 12.5, from about 13.2 to about 13.4, about 14.3 to about 14.5, from about 17.5 to about 17.7, and from about 26.1 to about 26.3 °2θ using Cu Kat radiation.

In some embodiments, the compound (e.g., the crystalline form of the hydrochloride salt of Compound 4R) is characterized by an XRPD pattern having a peak at about 7.20, about 7.95, about 8.77, about 9.78, about 12.37, about 13.26, about 14.41, about 17.60, and about 26.22 °2θ using Cu Kα radiation.

In some embodiments, the compound (e.g., the crystalline form of the hydrochloride salt of Compound 4R) has an endothermic peak top temperature in differential scanning calorimeter (DSC) analysis at between about 80° C. and about 120° C., between about 85° C. and about 115° C., between about 90° C. and about 110° C., between about 95° C. and about 105° C., or between about 99° C. and about 101° C.

In some embodiments, the compound (e.g., the crystalline form of the hydrochloride salt of Compound 4R) has an endothermic peak top temperature in differential scanning calorimeter (DSC) analysis at between about 220° C. and about 260° C., between about 225° C. and about 255° C., between about 230° C. and about 250° C., between about 235° C. and about 245° C., or between about 239° C. and about 240° C.

In some embodiments, the compound (e.g., the crystalline form of the hydrochloride salt of Compound 4R) has an endothermic peak top temperature in differential scanning calorimeter (DSC) analysis at about 100° C. and/or at about 239.2° C.

### Compound 41R Succinate Salt Type A

In some embodiments, the compound is a succinate salt of Compound 4R.

In some embodiments, the compound is a crystalline form of a succinate salt of Compound 4R.

In some embodiments, the compound (e.g., the crystalline form of the succinate salt of Compound 4R) is characterized by an XRPD pattern having at least one peak selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least two peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least three peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least four peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least five peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least six peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least seven peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least eight peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having at least nine peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having one peak selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having two peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having three peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having four peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having five peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having six peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having seven peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having eight peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having nine peaks selected from 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having a peak at 6.3±0.2, 6.8±0.2, 9.2±0.2, 12.7±0.2, 13.1±0.2, 14.4±0.2, 20.1±0.2, 22.0±0.2, 26.2±0.2, and 27.1±0.2 °2θ (e.g., 6.3±0.1, 6.8±0.1, 9.2±0.1, 12.7±0.1, 13.1±0.1, 14.4±0.1, 20.1±0.1, 22.0±0.1, 26.2±0.1, and 27.1±0.1 °2θ) using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, and from about 9.0 to about 9.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, and from about 12.9 to about 13.3 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.9 to about 13.3, and from about 26.0 to about 26.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.5 to about 12.9, from about 12.9 to about 13.3, and from about 26.0 to about 26.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.5 to about 12.9, from about 12.9 to about 13.3, from about 19.9 to about 20.3, and from about 26.0 to about 26.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.5 to about 12.9, from about 12.9 to about 13.3, from about 14.2 to about 14.6, from about 19.9 to about 20.3, and from about 26.0 to about 26.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.5 to about 12.9, from about 12.9 to about 13.3, from about 14.2 to about 14.6, from about 19.9 to about 20.3, from about 21.8 to about 22.2, and from about 26.0 to about 26.4 °2θ using Cu Kα radiation.

In some embodiments, the compound (e.g., the crystalline form of the succinate salt of Compound 4R) is characterized by an XRPD pattern having a peak at from about 6.1 to about 6.5, from about 6.6 to about 7.0, from about 9.0 to about 9.4, from about 12.5 to about 12.9, from about 12.9 to about 13.3, from about 14.2 to about 14.6, from about 19.9 to about 20.3, from about 21.8 to about 22.2, from about 26.0 to about 26.4, and from about 26.9 to about 27.3 °2θ using Cu Kα radiation.

In some embodiments, the compound (e.g., the crystalline form of the succinate salt of Compound 4R) is characterized by an XRPD pattern having a peak at from about 6.2 to about 6.4, from about 6.7 to about 6.9, from about 9.1 to about 9.3, from about 12.6 to about 12.8, from about 13.0 to about 13.2, from about 14.3 to about 14.5, from about 20.0 to about 20.2, from about 21.9 to about 22.1, from about 26.1 to about 26.3, and from about 27.0 to about 27.2 °2θ using Cu Kα radiation.

In some embodiments, the compound (e.g., the crystalline form of the succinate salt of Compound 4R) is characterized by an XRPD pattern having a peak at about 6.31, about 6.79, about 9.24, about 12.66, about 13.13, about 14.37, about 20.08, about 22.00, about 26.15, and about 27.05 °2θ using Cu Kα radiation.

In some embodiments, the compound (e.g., the crystalline form of the succinate salt of Compound 4R) has an endothermic peak top temperature in differential scanning calorimeter (DSC) analysis at between about 70° C. and about 110° C., between about 75° C. and about 105° C., between about 80° C. and about 100° C., between about 85° C. and about 95° C., or between about 88° C. and about 89° C.

In some embodiments, the compound (e.g., the crystalline form of the succinate salt of Compound 4R) has an endothermic peak top temperature in differential scanning calorimeter (DSC) analysis at between about 125° C. and about 165° C., between about 130° C. and about 160° C., between about 135° C. and about 155° C., between about 140° C. and about 150° C., or between about 146° C. and about 148° C.

In some embodiments, the compound (e.g., the crystalline form of the succinate salt of Compound 4R) has an endothermic peak top temperature in differential scanning calorimeter (DSC) analysis at between about 175° C. and about 215° C., between about 180° C. and about 210° C., between about 185° C. and about 205° C., between about 190° C. and about 200° C., or between about 193° C. and about 194° C.

In some embodiments, the compound (e.g., the crystalline form of the succinate salt of Compound 4R) has an endothermic peak top temperature in differential scanning calorimeter (DSC) analysis at between about 210° C. and about 250° C., between about 215° C. and about 245° C., between about 220° C. and about 240° C., between about 225° C. and about 235° C., or between about 231° C. and about 233° C.

In some embodiments, the compound (e.g., the crystalline form of the succinate salt of Compound 4R) has an endothermic peak top temperature in differential scanning calorimeter (DSC) analysis at about 88.7° C., at about 147.0° C., at about 193.6° C., and/or at about 232.0° C.

In some embodiments, the compound is Compound 4S, a tautomer thereof, a pharmaceutically acceptable salt thereof, or a pharmaceutically acceptable salt of the tautomer.

In some embodiments, the compound is Compound 4S.

In some embodiments, the compound is a crystalline form of Compound 4S.

In some embodiments, the compound is a pharmaceutically acceptable salt of Compound 4S.

In some embodiments, the compound is a crystalline form of a pharmaceutically acceptable salt of Compound 4S.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 4S.

In some embodiments, the compound is a hydrochloride salt of Compound 4S.

In some embodiments, the compound is a crystalline form of a hydrochloride salt of Compound 4S.

In some embodiments, the compound is a succinate salt of Compound 4R.

In some embodiments, the compound is a crystalline form of a succinate salt of Compound 4R.

### Compound 5

In some embodiments, the compound is

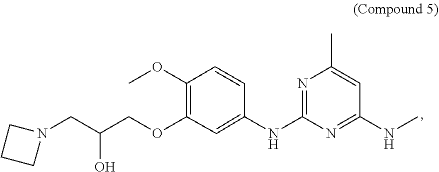 [SMILES: CNc1cc(C)nc(Nc2ccc(OC)c(OCC(O)CN3CCC3)c2)n1]

a tautomer thereof, a pharmaceutically acceptable salt thereof, or a pharmaceutically acceptable salt of the tautomer.

In some embodiments, the compound is Compound 5.

In some embodiments, the compound is

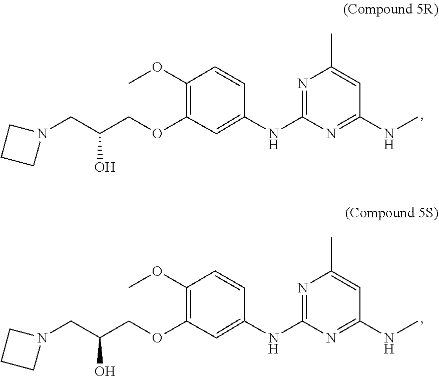 [SMILES: CNc1cc(C)nc(Nc2ccc(OC)c(OC[C@H](O)CN3CCC3)c2)n1 ; CNc1cc(C)nc(Nc2ccc(OC)c(OC[C@@H](O)CN3CCC3)c2)n1]

a tautomer thereof, a pharmaceutically acceptable salt thereof, or a pharmaceutically acceptable salt of the tautomer.

In some embodiments, the compound is Compound 5R or Compound 5S.

In some embodiments, the compound is Compound 5R, a tautomer thereof, a pharmaceutically acceptable salt thereof, or a pharmaceutically acceptable salt of the tautomer.

In some embodiments, the compound is Compound 5R.

In some embodiments, the compound is a crystalline form of Compound 5R.

In some embodiments, the crystalline form of Compound 5R is an anhydrate.

In some embodiments, the compound is a pharmaceutically acceptable salt of Compound 5R.

In some embodiments, the compound is a crystalline form of a pharmaceutically acceptable salt of Compound 5R.

In some embodiments, the crystalline form of the pharmaceutically acceptable salt of Compound 5R is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 5R.

### Compound 5R Freebase Type A

In some embodiments, the compound is Compound 5R.

In some embodiments, the compound is a crystalline form of Compound 5R.

In some embodiments, the compound (e.g., the crystalline form of Compound 5R) is characterized by an XRPD pattern having at least one peak selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having at least two peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having at least three peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having at least four peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having at least five peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having one peak selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having two peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having three peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having four peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having five peaks selected from 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having a peak at 12.8±0.2, 13.4±0.2, 14.6±0.2, 17.6±0.2, 20.9±0.2, and 23.9±0.2 °2θ (e.g., 12.8±0.1, 13.4±0.1, 14.6±0.1, 17.6±0.1, 20.9±0.1, and 23.9±0.1 °2θ) using Cu Kα radiation.

In some embodiments, the compound (e.g., the crystalline form of Compound 5R) is characterized by an XRPD pattern having a peak at from about 12.6 to about 13.0, from about 13.1 to about 13.6, and from about 20.7 to about 30.1 °2θ using Cu Kα radiation.

In some embodiments, the compound (e.g., the crystalline form of Compound 5R) is characterized by an XRPD pattern having a peak at from about 12.6 to about 13.0, from about 13.1 to about 13.6, from about 17.4 to about 17.8, and from about 20.7 to about 30.1 °2θ using Cu Kα radiation.

In some embodiments, the compound (e.g., the crystalline form of Compound 5R) is characterized by an XRPD pattern having a peak at from about 12.6 to about 13.0, from about 13.1 to about 13.6, from about 17.4 to about 17.8, from about 20.7 to about 30.1, and from about 23.8 to about 24.0 °2θ using Cu Kα radiation.

In some embodiments, the compound (e.g., the crystalline form of Compound 5R) is characterized by an XRPD pattern having a peak at from about 12.6 to about 13.0, from about 13.1 to about 13.6, from about 14.4 to about 14.8, from about 17.4 to about 17.8, from about 20.7 to about 30.1, and from about 23.8 to about 24.0 °2θ using Cu Kα radiation.

In some embodiments, the compound (e.g., the crystalline form of Compound 5R) is characterized by an XRPD pattern having a peak at from about 12.7 to about 12.9, from about 13.3 to about 13.5, from about 14.5 to about 14.7, from about 17.5 to about 17.7, from about 20.8 to about 30.0, and from about 23.7 to about 24.1 °2θ using Cu Kao radiation.

In some embodiments, the compound (e.g., the crystalline form of Compound 5R) is characterized by an XRPD pattern having a peak at about 12.81, about 13.39, about 14.57, about 17.55, about 20.85, and about 23.91 °2θ using Cu Kα radiation.

In some embodiments, the compound (e.g., the crystalline form of Compound 5R) has an endothermic peak top temperature in differential scanning calorimeter (DSC) analysis at between about 90° C. and about 130° C., between about 95° C. and about 125° C., between about 100° C. and about 120° C., between about 105° C. and about 115° C., or between about 109° C. and about 112° C.

In some embodiments, the compound (e.g., the crystalline form of Compound 5R) has an endothermic peak top temperature in differential scanning calorimeter (DSC) analysis at between about 120° C. and about 160° C., between about 125° C. and about 155° C., between about 130° C. and about 150° C., between about 135° C. and about 145° C., or between about 140° C. and about 142° C.

In some embodiments, the compound (e.g., the crystalline form of Compound 5R) has an endothermic peak top temperature in differential scanning calorimeter (DSC) analysis at about 110.5° C. and/or at about 141.0° C.

### Compound 5R Freebase Type B

In some embodiments, the compound is Compound 5R.

In some embodiments, the compound is a crystalline form of Compound 5R.

In some embodiments, the compound (e.g., the crystalline form of Compound 5R) is characterized by an XRPD pattern having at least one peak selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having at least two peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having at least three peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having at least four peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having at least five peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having at least six peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having one peak selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having two peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having three peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having four peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having five peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having six peaks selected from 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having a peak at 10.2±0.2, 12.5±0.2, 14.0±0.2, 17.8±0.2, 18.8±0.2, 19.3±0.2, and 24.6±0.2 °2θ (e.g., 10.2±0.1, 12.5±0.1, 14.0±0.1, 17.8±0.1, 18.8±0.1, 19.3±0.1, and 24.6±0.1 °2θ) using Cu Kα radiation.

In some embodiments, the compound (e.g., the crystalline form of Compound 5R) is characterized by an XRPD pattern having a peak at from about 13.8 to about 14.2, from about 17.6 to about 18.0, and from about 18.6 to about 19.0 °2θ using Cu Kα radiation.

In some embodiments, the compound (e.g., the crystalline form of Compound 5R) is characterized by an XRPD pattern having a peak at from about 12.3 to about 12.7, from about 13.8 to about 14.2, from about 17.6 to about 18.0, and from about 18.6 to about 19.0 °2θ using Cu Kα radiation.

In some embodiments, the compound (e.g., the crystalline form of Compound 5R) is characterized by an XRPD pattern having a peak at from about 12.3 to about 12.7, from about 13.8 to about 14.2, from about 17.6 to about 18.0, from about 18.6 to about 19.0, and about from about 19.1 to about 19.5 °2θ using Cu Kα radiation.

In some embodiments, the compound (e.g., the crystalline form of Compound 5R) is characterized by an XRPD pattern having a peak at from about 12.3 to about 12.7, from about 13.8 to about 14.2, from about 17.6 to about 18.0, from about 18.6 to about 19.0, from about 19.1 to about 19.5, and from about 24.4 to about 24.8 °2θ using Cu Kα radiation.

In some embodiments, the compound (e.g., the crystalline form of Compound 5R) is characterized by an XRPD pattern having a peak at from about 10.0 to about 10.4, from about 12.3 to about 12.7, from about 13.8 to about 14.2, from about 17.6 to about 18.0, from about 18.6 to about 19.0, from about 19.1 to about 19.5, and from about 24.4 to about 24.8 °2θ using Cu Kα radiation.

In some embodiments, the compound (e.g., the crystalline form of Compound 5R) is characterized by an XRPD pattern having a peak at from about 10.1 to about 10.3, from about 12.4 to about 12.6, from about 13.9 to about 14.1, from about 17.7 to about 17.9, from about 18.7 to about 18.9, from about 19.2 to about 19.4, and from about 24.5 to about 24.7 °2θ using Cu Kα radiation.

In some embodiments, the compound (e.g., the crystalline form of Compound 5R) is characterized by an XRPD pattern having a peak at about 10.17, about 12.49, about 13.97, about 17.75, about 18.82, about 19.34, and about 24.56 °2θ using Cu Kα radiation.

In some embodiments, the compound (e.g., the crystalline form of Compound 5R) has an endothermic peak top temperature in differential scanning calorimeter (DSC) analysis at between about 120° C. and about 160° C., between about 125° C. and about 155° C., between about 130° C. and about 150° C., between about 135° C. and about 145° C., or between about 138° C. and about 141° C.

In some embodiments, the compound (e.g., the crystalline form of Compound 5R) has an endothermic peak top temperature in differential scanning calorimeter (DSC) analysis at about 139.5° C.

### Compound 5R Freebase Type C

In some embodiments, the compound is Compound 5R.

In some embodiments, the compound is a crystalline form of Compound 5R.

In some embodiments, the compound (e.g., the crystalline form of Compound 5R) is characterized by an XRPD pattern having at least one peak selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least two peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least three peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least four peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least five peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1 and 24.8±0.1 °2θ) using Cu Kα radiation.

In some embodiments, the compound (e.g., the crystalline form of Compound 5R) is characterized by an XRPD pattern having at least six peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least seven peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least eight peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having at least nine peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having one peak selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having two peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having three peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having four peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having five peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having six peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having seven peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having eight peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having nine peaks selected from 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having a peak at 8.5±0.2, 12.9±0.2, 13.6±0.2, 15.4±0.2, 16.0±0.2, 18.1±0.2, 21.3±0.2, 21.6±0.2, 22.9±0.2, and 24.8±0.2 °2θ (e.g., 8.5±0.1, 12.9±0.1, 13.6±0.1, 15.4±0.1, 16.0±0.1, 18.1±0.1, 21.3±0.1, 21.6±0.1, 22.9±0.1, and 24.8±0.1 °2θ) using Cu Kα radiation.

In some embodiments, the compound (e.g., the crystalline form of Compound 5R) is characterized by an XRPD pattern having a peak at from about 8.3 to about 8.7, from about 12.7 to about 13.1, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, from about 17.9 to about 18.3, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, from about 17.9 to about 18.3, from about 21.1 to about 21.5, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, from about 15.8 to about 16.2, from about 17.9 to about 18.3, from about 21.1 to about 21.5, and from about 21.4 to about 21.8 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, from about 15.8 to about 16.2, from about 17.9 to about 18.3, from about 21.1 to about 21.5, from about 21.4 to about 21.8, and from about 22.7 to about 23.1 °2θ using Cu Kα radiation.

In some embodiments, the compound (e.g., the crystalline form of Compound 5R) is characterized by an XRPD pattern having a peak at from about 8.3 to about 8.7, from about 12.7 to about 13.1, from about 13.4 to about 13.8, from about 15.2 to about 15.6, from about 15.8 to about 16.2, from about 17.9 to about 18.3, from about 21.1 to about 21.5, from about 21.4 to about 21.8, from about 22.7 to about 23.1, and from about 24.6 to about 25.0 °2θ using Cu Kα radiation.

In some embodiments, the compound (e.g., the crystalline form of Compound 5R) is characterized by an XRPD pattern having a peak at from about 8.4 to about 8.6, from about 12.8 to about 13.0, from about 13.5 to about 13.7, from about 15.3 to about 15.5, from about 15.9 to about 16.1, from about 18.0 to about 18.2, from about 21.2 to about 21.4, from about 21.5 to about 21.7, from about 22.8 to about 23.0, and from about 24.7 to about 24.9 °2θ using Cu Kα radiation.

In some embodiments, the compound (e.g., the crystalline form of Compound 5R) is characterized by an XRPD pattern having a peak at about 8.48, about 12.86, about 13.55, about 15.41, about 16.01, about 18.14, about 21.32, about 21.63, about 22.87, and about 24.84 °2θ using Cu Kα radiation.

### Compound 5R Sulfate Salt Type A

In some embodiments, the compound is a sulfate salt of Compound 5R.

In some embodiments, the compound is a crystalline form of a sulfate salt of Compound 5R.

In some embodiments, the compound (e.g., the crystalline form of the sulfate salt of Compound 5R) is characterized by an XRPD pattern having at least one peak selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having at least two peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having at least three peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having at least four peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having at least five peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having at least six peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having one peak selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having two peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having three peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having four peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having five peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having six peaks selected from 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having a peak at 6.8±0.2, 8.7±0.2, 14.0±0.2, 16.4±0.2, 23.5±0.2, 25.3±0.2, and 26.5±0.2 °2θ (e.g., 6.8±0.1, 8.7±0.1, 14.0±0.1, 16.4±0.1, 23.5±0.1, 25.3±0.1, and 26.5±0.1 °2θ) using Cu Kα radiation.

In some embodiments, the compound (e.g., the crystalline form of the sulfate salt of Compound 5R) is characterized by an XRPD pattern having a peak at from about 8.5 to about 8.9, from about 13.8 to about 14.2, and from about 16.2 to about 16.6 °2θ using Cu Kα radiation.

In some embodiments, the compound (e.g., the crystalline form of the sulfate salt of Compound 5R) is characterized by an XRPD pattern having a peak at from about 8.5 to about 8.9, from about 13.8 to about 14.2, from about 16.2 to about 16.6, and from about 26.3 to about 26.7 °2θ using Cu Kα radiation.

In some embodiments, the compound (e.g., the crystalline form of the sulfate salt of Compound 5R) is characterized by an XRPD pattern having a peak at from about 8.5 to about 8.9, from about 13.8 to about 14.2, from about 16.2 to about 16.6, from about 25.1 to about 25.5, and from about 26.3 to about 26.7 °2θ using Cu Kα radiation.

In some embodiments, the compound (e.g., the crystalline form of the sulfate salt of Compound 5R) is characterized by an XRPD pattern having a peak at from about 6.6 to about 7.0, from about 8.5 to about 8.9, from about 13.8 to about 14.2, from about 16.2 to about 16.6, from about 25.1 to about 25.5, and from about 26.3 to about 26.7 °2θ using Cu Kα radiation.

In some embodiments, the compound (e.g., the crystalline form of the sulfate salt of Compound 5R) is characterized by an XRPD pattern having a peak at from about 6.6 to about 7.0, from about 8.5 to about 8.9, from about 13.8 to about 14.2, from about 16.2 to about 16.6, from about 23.3 to about 23.7, from about 25.1 to about 25.5, and from about 26.3 to about 26.7 °2θ using Cu Kα radiation.

In some embodiments, the compound (e.g., the crystalline form of the sulfate salt of Compound 5R) is characterized by an XRPD pattern having a peak at from about 6.7 to about 6.9, from about 8.6 to about 8.8, from about 13.9 to about 14.1, from about 16.3 to about 16.5, from about 23.4 to about 23.6, from about 25.2 to about 25.4, and from about 26.4 to about 26.6 °2θ using Cu Kα radiation.

In some embodiments, the compound (e.g., the crystalline form of the sulfate salt of Compound 5R) is characterized by an XRPD pattern having a peak at about 6.77, about 8.65, about 13.95, about 16.42, about 23.49, about 25.29, and about 26.50 °2θ using Cu Kα radiation.

### Compound 5R Glycolate Salt Type A

In some embodiments, the compound is a glycolate salt of Compound 5R.

In some embodiments, the compound is a crystalline form of a glycolate salt of Compound 5R.

In some embodiments, the compound (e.g., the crystalline form of the glycolate salt of Compound 5R) is characterized by an XRPD pattern having at least one peak selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having at least two peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having at least three peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having at least four peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having at least five peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having at least six peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having one peak selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having two peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having three peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having four peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having five peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having six peaks selected from 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having a peak at 6.5±0.2, 14.1±0.2, 17.8±0.2, 18.9±0.2, 24.7±0.2, 25.7±0.2, and 26.5±0.2 °2θ (e.g., 6.5±0.1, 14.1±0.1, 17.8±0.1, 18.9±0.1, 24.7±0.1, 25.7±0.1, and 26.5±0.1 °2θ) using Cu Kα radiation.

In some embodiments, the compound (e.g., the crystalline form of the glycolate salt of Compound 5R) is characterized by an XRPD pattern having a peak at from about 6.3 to about 6.7, from about 17.6 to about 18.0, and from about 18.7 to about 19.1 °2θ using Cu Kα radiation.

In some embodiments, the compound (e.g., the crystalline form of the glycolate salt of Compound 5R) is characterized by an XRPD pattern having a peak at from about 6.3 to about 6.7, from about 17.6 to about 18.0, from about 18.7 to about 19.1, and from about 26.3 to about 26.7 °2θ using Cu Kα radiation.

In some embodiments, the compound (e.g., the crystalline form of the glycolate salt of Compound 5R) is characterized by an XRPD pattern having a peak at from about 6.3 to about 6.7, from about 17.6 to about 18.0, from about 18.7 to about 19.1, from about 25.5 to about 25.9, and from about 26.3 to about 26.7 °2θ using Cu Kα radiation.

In some embodiments, the compound (e.g., the crystalline form of the glycolate salt of Compound 5R) is characterized by an XRPD pattern having a peak at from about 6.3 to about 6.7, from about 17.6 to about 18.0, from about 18.7 to about 19.1, from about 24.5 to about 24.9, from about 25.5 to about 25.9, and from about 26.3 to about 26.7 °2θ using Cu Kα radiation.

In some embodiments, the compound (e.g., the crystalline form of the glycolate salt of Compound 5R) is characterized by an XRPD pattern having a peak at from about 6.3 to about 6.7, from about 13.9 to about 14.3, from about 17.6 to about 18.0, from about 18.7 to about 19.1, from about 24.5 to about 24.9, from about 25.5 to about 25.9, and from about 26.3 to about 26.7 °2θ using Cu Kα radiation.

In some embodiments, the compound (e.g., the crystalline form of the glycolate salt of Compound 5R) is characterized by an XRPD pattern having a peak at from about 6.4 to about 6.6, from about 14.0 to about 14.2, from about 17.7 to about 17.9, from about 18.8 to about 19.0, from about 24.6 to about 24.8, from about 25.6 to about 25.8, and from about 26.4 to about 26.6 °2θ using Cu Kα radiation.

In some embodiments, the compound (e.g., the crystalline form of the glycolate salt of Compound 5R) is characterized by an XRPD pattern having a peak at about 6.52, about 14.06, about 17.83, about 18.94, about 24.69, about 25.67, and about 26.49 °2θ using Cu Kα radiation.

### Compound 5R Fumarate Salt Type A

In some embodiments, the compound is a fumarate salt of Compound 5R.

In some embodiments, the compound is a crystalline form of a fumarate salt of Compound 5R.

In some embodiments, the compound (e.g., the crystalline form of the fumarate salt of Compound 5R) is characterized by an XRPD pattern having at least one peak selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least two peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least three peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least four peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least five peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least six peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having at least seven peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having one peak selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kao radiation.

In some embodiments, the compound (e.g., the crystalline form of the fumarate salt of Compound 5R) is characterized by an XRPD pattern having two peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having three peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having four peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having five peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having six peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having seven peaks selected from 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having a peak at 5.9±0.2, 7.7±0.2, 11.3±0.2, 11.9±0.2, 15.4±0.2, 18.4±0.2, 25.8±0.2, and 26.5±0.2 °2θ (e.g., 5.9±0.1, 7.7±0.1, 11.3±0.1, 11.9±0.1, 15.4±0.1, 18.4±0.1, 25.8±0.1, and 26.5±0.1 °2θ) using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, from about 15.2 to about 15.6, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, from about 15.2 to about 15.6, from about 25.6 to about 26.0, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, from about 15.2 to about 15.6, from about 18.2 to about 18.6, from about 25.6 to about 26.0, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, from about 11.7 to about 12.1, from about 15.2 to about 15.6, from about 18.2 to about 18.6, from about 25.6 to about 26.0, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.7 to about 6.1, from about 7.5 to about 7.9, from about 11.1 to about 11.5, from about 11.7 to about 12.1, from about 15.2 to about 15.6, from about 18.2 to about 18.6, from about 25.6 to about 26.0, and from about 26.3 to about 26.7 °2θ using Cu Kα radiation.

In some embodiments, the compound (e.g., the crystalline form of the fumarate salt of Compound 5R) is characterized by an XRPD pattern having a peak at from about 5.8 to about 6.0, from about 7.6 to about 7.8, from about 11.2 to about 11.4, from about 11.8 to about 12.0, from about 15.3 to about 15.5, from about 18.3 to about 18.5, from about 25.7 to about 25.9, and from about 26.4 to about 26.6 °2θ using Cu Kα radiation.

In some embodiments, the compound (e.g., the crystalline form of the fumarate salt of Compound 5R) is characterized by an XRPD pattern having a peak at about 5.94, about 7.66, about 11.31, about 11.88, about 15.40, about 18.41, about 25.84, and about 26.47 °2θ using Cu Kα radiation.

### Compound 5R Hippurate Salt Type A

In some embodiments, the compound is a hippurate salt of Compound 5R.

In some embodiments, the compound is a crystalline form of a hippurate salt of Compound 5R.

In some embodiments, the compound (e.g., the crystalline form of the hippurate salt of Compound 5R) is characterized by an XRPD pattern having at least one peak selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having at least two peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having at least three peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having at least four peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having at least five peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having at least six peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having one peak selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having two peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having three peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having four peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having five peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having six peaks selected from 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having a peak at 6.5±0.2, 9.7±0.2, 11.0±0.2, 13.0±0.2, 19.4±0.2, 23.6±0.2, and 26.1±0.2 °2θ (e.g., 6.5±0.1, 9.7±0.1, 11.0±0.1, 13.0±0.1, 19.4±0.1, 23.6±0.1, and 26.1±0.1 °2θ) using Cu Kα radiation.

In some embodiments, the compound (e.g., the crystalline form of the hippurate salt of Compound 5R) is characterized by an XRPD pattern having a peak at from about 6.3 to about 6.7, from about 12.8 to about 13.2, and from about 25.9 to about 26.3 °2θ using Cu Kα radiation.

In some embodiments, the compound (e.g., the crystalline form of the hippurate salt of Compound 5R) is characterized by an XRPD pattern having a peak at from about 6.3 to about 6.7, from about 12.8 to about 13.2, from about 19.2 to about 19.6, and from about 25.9 to about 26.3 °2θ using Cu Kα radiation.

In some embodiments, the compound (e.g., the crystalline form of the hippurate salt of Compound 5R) is characterized by an XRPD pattern having a peak at from about 6.3 to about 6.7, from about 12.8 to about 13.2, from about 19.2 to about 19.6, from about 23.4 to about 23.8, and from about 25.9 to about 26.3 °2θ using Cu Kα radiation.

In some embodiments, the compound (e.g., the crystalline form of the hippurate salt of Compound 5R) is characterized by an XRPD pattern having a peak at from about 6.3 to about 6.7, from about 9.5 to about 9.9, from about 12.8 to about 13.2, from about 19.2 to about 19.6, from about 23.4 to about 23.8, and from about 25.9 to about 26.3 °2θ using Cu Kα radiation.

In some embodiments, the compound (e.g., the crystalline form of the hippurate salt of Compound 5R) is characterized by an XRPD pattern having a peak at from about 6.3 to about 6.7, from about 9.5 to about 9.9, from about 10.8 to about 11.2, from about 12.8 to about 13.2, from about 19.2 to about 19.6, from about 23.4 to about 23.8, and from about 25.9 to about 26.3 °2θ using Cu Kα radiation.

In some embodiments, the compound (e.g., the crystalline form of the hippurate salt of Compound 5R) is characterized by an XRPD pattern having a peak at from about 6.4 to about 6.6, from about 9.6 to about 9.8, from about 10.9 to about 11.1, from about 12.9 to about 13.1, from about 19.3 to about 19.5, from about 23.5 to about 23.7, and from about 26.0 to about 26.2 °2θ using Cu Kα radiation.

In some embodiments, the compound (e.g., the crystalline form of the hippurate salt of Compound 5R) is characterized by an XRPD pattern having a peak at about 6.49, about 9.70, about 10.98, about 12.96, about 19.44, about 23.62, and about 26.07 °2θ using Cu Kα radiation.

### Compound 5R Adipate Salt Type A

In some embodiments, the compound is an adipate salt of Compound 5R.

In some embodiments, the compound is a crystalline form of an adipate salt of Compound 5R.

In some embodiments, the compound (e.g., the crystalline form of the adipate salt of Compound 5R) is characterized by an XRPD pattern having at least one peak selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least two peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least three peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least four peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least five peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least six peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having at least seven peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having one peak selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having two peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having three peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having four peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having five peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having six peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having seven peaks selected from 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having a peak at 10.7±0.2, 13.1±0.2, 17.8±0.2, 18.8±0.2, 21.6±0.2, 22.9±0.2, 24.6±0.2, and 25.5±0.2 °2θ (e.g., 10.7±0.1, 13.1±0.1, 17.8±0.1, 18.8±0.1, 21.6±0.1, 22.9±0.1, 24.6±0.1, and 25.5±0.1 °2θ) using Cu Kα radiation.

In some embodiments, the compound (e.g., the crystalline form of the adipate salt of Compound 5R) is characterized by an XRPD pattern having a peak at from about 21.4 to about 21.8, from about 22.7 to about 23.1, and from about 25.3 to about 25.7 °2θ using Cu Kα radiation.

In some embodiments, the compound (e.g., the crystalline form of the adipate salt of Compound 5R) is characterized by an XRPD pattern having a peak at from about 12.9 to about 13.3, from about 21.4 to about 21.8, from about 22.7 to about 23.1, and from about 25.3 to about 25.7 °2θ using Cu Kα radiation.

In some embodiments, the compound (e.g., the crystalline form of the adipate salt of Compound 5R) is characterized by an XRPD pattern having a peak at from about 12.9 to about 13.3, from about 17.6 to about 18.0, from about 21.4 to about 21.8, from about 22.7 to about 23.1, and from about 25.3 to about 25.7 °2θ using Cu Kα radiation.

In some embodiments, the compound (e.g., the crystalline form of the adipate salt of Compound 5R) is characterized by an XRPD pattern having a peak at from about 10.5 to about 10.9, from about 12.9 to about 13.3, from about 17.6 to about 18.0, from about 21.4 to about 21.8, from about 22.7 to about 23.1, and from about 25.3 to about 25.7 °2θ using Cu Kα radiation.

In some embodiments, the compound (e.g., the crystalline form of the adipate salt of Compound 5R) is characterized by an XRPD pattern having a peak at from about 10.5 to about 10.9, from about 12.9 to about 13.3, from about 17.6 to about 18.0, from about 18.6 to about 19.0, from about 21.4 to about 21.8, from about 22.7 to about 23.1, and from about 25.3 to about 25.7 °2θ using Cu Kα radiation.

In some embodiments, the compound (e.g., the crystalline form of the adipate salt of Compound 5R) is characterized by an XRPD pattern having a peak at from about 10.5 to about 10.9, from about 12.9 to about 13.3, from about 17.6 to about 18.0, from about 18.6 to about 19.0, from about 21.4 to about 21.8, from about 22.7 to about 23.1, from about 24.4 to about 24.8, and from about 25.3 to about 25.7 °2θ using Cu Kao radiation.

In some embodiments, the compound (e.g., the crystalline form of the adipate salt of Compound 5R) is characterized by an XRPD pattern having a peak at from about 10.6 to about 10.8, from about 13.0 to about 13.2, from about 17.7 to about 17.9, from about 18.7 to about 18.9, from about 21.5 to about 21.7, from about 22.8 to about 23.0, from about 24.5 to about 24.7, and from about 25.4 to about 25.6 °2θ using Cu Kα radiation.

In some embodiments, the compound (e.g., the crystalline form of the adipate salt of Compound 5R) is characterized by an XRPD pattern having a peak at about 10.66, about 13.06, about 17.78, about 18.84, about 21.55, about 22.89, about 24.55, and about 25.45 °2θ using Cu Kα radiation.

### Compound 5R Gentisate Salt Type A

In some embodiments, the compound is a gentisate salt of Compound 5R.

In some embodiments, the compound is a crystalline form of a gentisate salt of Compound 5R.

In some embodiments, the compound (e.g., the crystalline form of the gentisate salt of Compound 5R) is characterized by an XRPD pattern having at least one peak selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least two peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least three peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least four peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least five peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least six peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least seven peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having at least eight peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having one peak selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having two peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having three peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having four peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having five peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having six peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having seven peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having eight peaks selected from 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having a peak at 5.3±0.2, 7.7±0.2, 8.8±0.2, 9.3±0.2, 15.0±0.2, 16.2±0.2, 17.2±0.2, 21.2±0.2, and 25.3±0.2 °2θ (e.g., 5.3±0.1, 7.7±0.1, 8.8±0.1, 9.3±0.1, 15.0±0.1, 16.2±0.1, 17.2±0.1, 21.2±0.1, and 25.3±0.1 °2θ) using Cu Kα radiation.

In some embodiments, the compound (e.g., the crystalline form of the gentisate salt of Compound 5R) is characterized by an XRPD pattern having a peak at from about 5.1 to about 5.5, from about 9.1 to about 9.5, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 8.6 to about 9.0, from about 9.1 to about 9.5, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 7.5 to about 7.9, from about 8.6 to about 9.0, from about 9.1 to about 9.5, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 7.5 to about 7.9, from about 8.6 to about 9.0, from about 9.1 to about 9.5, from about 16.0 to about 16.4, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 7.5 to about 7.9, from about 8.6 to about 9.0, from about 9.1 to about 9.5, from about 16.0 to about 16.4, from about 17.0 to about 17.4, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 7.5 to about 7.9, from about 8.6 to about 9.0, from about 9.1 to about 9.5, from about 14.8 to about 15.2, from about 16.0 to about 16.4, from about 17.0 to about 17.4, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.1 to about 5.5, from about 7.5 to about 7.9, from about 8.6 to about 9.0, from about 9.1 to about 9.5, from about 14.8 to about 15.2, from about 16.0 to about 16.4, from about 17.0 to about 17.4, from about 21.1 to about 21.5, and from about 25.1 to about 25.5 °2θ using Cu Kα radiation.

In some embodiments, the compound (e.g., the crystalline form of the gentisate salt of Compound 5R) is characterized by an XRPD pattern having a peak at from about 5.2 to about 5.4, from about 7.6 to about 7.8, from about 8.7 to about 8.9, from about 9.2 to about 9.4, from about 14.9 to about 15.1, from about 16.1 to about 16.3, from about 17.1 to about 17.3, from about 21.2 to about 21.4, and from about 25.2 to about 25.4 °2θ using Cu Kα radiation.

In some embodiments, the compound (e.g., the crystalline form of the gentisate salt of Compound 5R) is characterized by an XRPD pattern having a peak at about 5.25, about 7.66, about 8.84, about 9.34, about 14.97, about 16.22, about 17.15, about 21.25, and about 25.26 °2θ using Cu Kα radiation.

### Compound 5R Gentisate Salt Type E

In some embodiments, the compound is a gentisate salt of Compound 5R.

In some embodiments, the compound is a crystalline form of a gentisate salt of Compound 5R.

In some embodiments, the compound (e.g., the crystalline form of the gentisate salt of Compound 5R) is characterized by an XRPD pattern having at least one peak selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least two peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least three peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least four peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least five peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least six peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least seven peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having at least eight peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having one peak selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having two peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having three peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having four peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having five peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having six peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having seven peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having eight peaks selected from 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having a peak at 6.0±0.2, 9.1±0.2, 15.0±0.2, 17.7±0.2, 18.4±0.2, 20.7±0.2, 23.8±0.2, 25.8±0.2, and 26.6±0.2 °2θ (e.g., 6.0±0.1, 9.1±0.1, 15.0±0.1, 17.7±0.1, 18.4±0.1, 20.7±0.1, 23.8±0.1, 25.8±0.1, and 26.6±0.1 °2θ) using Cu Kα radiation.

In some embodiments, the compound (e.g., the crystalline form of the gentisate salt of Compound 5R) is characterized by an XRPD pattern having a peak at from about 5.8 to about 6.2, from about 14.8 to about 15.2, and from about 18.2 to about 18.6 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, and from about 18.2 to about 18.6 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, from about 18.2 to about 18.6, and from about 20.5 to about 20.9 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, from about 18.2 to about 18.6, from about 20.5 to about 20.9, and from about 26.4 to about 26.8 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, from about 17.5 to about 17.9, from about 18.2 to about 18.6, from about 20.5 to about 20.9, and from about 26.4 to about 26.8 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, from about 17.5 to about 17.9, from about 18.2 to about 18.6, from about 20.5 to about 20.9, from about 25.6 to about 26.0, and from about 26.4 to about 26.8 °2θ using Cu Kα radiation.

In some embodiments, the compound (e.g., the crystalline form of the gentisate salt of Compound 5R) is characterized by an XRPD pattern having a peak at from about 5.8 to about 6.2, from about 8.9 to about 9.3, from about 14.8 to about 15.2, from about 17.5 to about 17.9, from about 18.2 to about 18.6, from about 20.5 to about 20.9, from about 23.6 to about 24.0, from about 25.6 to about 26.0, and from about 26.4 to about 26.8 °2θ using Cu Kα radiation.

In some embodiments, the compound (e.g., the crystalline form of the gentisate salt of Compound 5R) is characterized by an XRPD pattern having a peak at from about 5.9 to about 6.1, from about 9.0 to about 9.2, from about 14.9 to about 15.1, from about 17.6 to about 17.8, from about 18.3 to about 18.5, from about 20.6 to about 20.8, from about 23.7 to about 23.9, from about 25.7 to about 25.9, and from about 26.5 to about 26.7 °2θ using Cu Kα radiation.

In some embodiments, the compound (e.g., the crystalline form of the gentisate salt of Compound 5R) is characterized by an XRPD pattern having a peak at about 6.01, about 9.13, about 15.02, about 17.74, about 18.41, about 20.72, about 23.77, about 25.84, and about 26.62 °2θ using Cu Kα radiation.

In some embodiments, the compound (e.g., the crystalline form of Compound 6) has an endothermic peak top temperature in differential scanning calorimeter (DSC) analysis at about 160° C. and about 200° C., between about 165° C. and about 195° C., between about 170° C. and about 190° C., between about 174° C. and about 185° C., or between about 178° C. and about 180° C.

In some embodiments, the compound (e.g., the crystalline form of the gentisate salt of Compound 5R) has an endothermic peak top temperature in differential scanning calorimeter (DSC) analysis at about 179° C.

### Compound 5R Benzoate Salt Type A

In some embodiments, the compound is a benzoate salt of Compound 5R.

In some embodiments, the compound is a crystalline form of a benzoate salt of Compound 5R.

In some embodiments, the compound (e.g., the crystalline form of the benzoate salt of Compound 5R) is characterized by an XRPD pattern having at least one peak selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least two peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least three peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least four peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least five peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least six peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 ° 28) using Cu Kα radiation.

In some embodiments, the compound (e.g., the crystalline form of the benzoate salt of Compound 5R) is characterized by an XRPD pattern having at least seven peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having at least eight peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having one peak selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having two peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having three peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having four peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having five peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having six peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having seven peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having eight peaks selected from 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having a peak at 5.2±0.2, 9.7±0.2, 15.5±0.2, 18.3±0.2, 19.0±0.2, 21.3±0.2, 22.9±0.2, 23.7±0.2, and 26.9±0.2 °2θ (e.g. 5.2±0.1, 9.7±0.1, 15.5±0.1, 18.3±0.1, 19.0±0.1, 21.3±0.1, 22.9±0.1, 23.7±0.1, and 26.9±0.1 °2θ) using Cu Kα radiation.

In some embodiments, the compound (e.g., the crystalline form of the benzoate salt of Compound 5R) is characterized by an XRPD pattern having a peak at from about 5.1 to about 5.5, from about 15.3 to about 15.7, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 15.3 to about 15.7, from about 18.8 to about 19.2, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 15.3 to about 15.7, from about 18.8 to about 19.2, from about 21.1 to about 21.5, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 9.5 to about 9.9, from about 15.3 to about 15.7, from about 18.8 to about 19.2, from about 21.1 to about 21.5, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 9.5 to about 9.9, from about 15.3 to about 15.7, from about 18.8 to about 19.2, from about 21.1 to about 21.5, from about 22.7 to about 23.1, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 9.5 to about 9.9, from about 15.3 to about 15.7, from about 18.8 to about 19.2, from about 21.1 to about 21.5, from about 22.7 to about 23.1, from about 23.5 to about 23.9, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.1 to about 5.5, from about 9.5 to about 9.9, from about 15.3 to about 15.7, from about 18.1 to about 18.5, from about 18.8 to about 19.2, from about 21.1 to about 21.5, from about 22.7 to about 23.1, from about 23.5 to about 23.9, and from about 26.7 to about 27.1 °2θ using Cu Kα radiation.

In some embodiments, the compound (e.g., the crystalline form of the benzoate salt of Compound 5R) is characterized by an XRPD pattern having a peak at from about 5.2 to about 5.4, from about 9.6 to about 9.8, from about 15.4 to about 15.6, from about 18.2 to about 18.4, from about 18.9 to about 19.1, from about 21.2 to about 21.4, from about 22.8 to about 23.0, from about 23.6 to about 23.8, and from about 26.8 to about 27.0 °2θ using Cu Kα radiation.

In some embodiments, the compound (e.g., the crystalline form of the benzoate salt of Compound 5R) is characterized by an XRPD pattern having a peak at about 5.28, about 9.66, about 15.51, about 18.25, about 19.03, about 21.27, about 22.91, about 23.73, and about 26.93 °2θ using Cu Kα radiation.

### Compound 5R Benzoate Salt Type B

In some embodiments, the compound is a benzoate salt of Compound 5R.

In some embodiments, the compound is a crystalline form of a benzoate salt of Compound 5R.

In some embodiments, the compound (e.g., the crystalline form of the benzoate salt of Compound 5R) is characterized by an XRPD pattern having at least one peak selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having at least two peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having at least three peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having at least four peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having at least five peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having one peak selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having two peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having three peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having four peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having five peaks selected from 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having a peak at 7.9±0.2, 10.1±0.2, 11.7±0.2, 17.2±0.2, 24.4±0.2, and 25.1±0.2 °2θ (e.g., 7.9±0.1, 10.1±0.1, 11.7±0.1, 17.2±0.1, 24.4±0.1, and 25.1±0.1 °2θ) using Cu Kα radiation.

In some embodiments, the compound (e.g., the crystalline form of the benzoate salt of Compound 5R) is characterized by an XRPD pattern having a peak at from about 17.0 to about 17.4, from about 24.2 to about 24.6, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 7.7 to about 8.1, from about 17.0 to about 17.4, from about 24.2 to about 24.6, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 7.7 to about 8.1, from about 11.5 to about 11.9, from about 17.0 to about 17.4, from about 24.2 to about 24.6, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 7.7 to about 8.1, from about 9.9 to about 10.3, from about 11.5 to about 11.9, from about 17.0 to about 17.4, from about 24.2 to about 24.6, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 7.8 to about 8.0, from about 10.0 to about 10.2, from about 11.6 to about 11.8, from about 17.1 to about 17.3, from about 24.3 to about 24.5, and from about 25.0 to about 25.2 °2θ using Cu Kα radiation.

In some embodiments, the compound (e.g., the crystalline form of the benzoate salt of Compound 5R) is characterized by an XRPD pattern having a peak at about 7.90, about 10.08, about 11.71, about 17.19, about 24.44, and about 25.13 °2θ using Cu Kα radiation.

### Compound 5R Benzoate Salt Type C

In some embodiments, the compound is a benzoate salt of Compound 5R.

In some embodiments, the compound is a crystalline form of a benzoate salt of Compound 5R.

In some embodiments, the compound (e.g., the crystalline form of the benzoate salt of Compound 5R) is characterized by an XRPD pattern having at least one peak selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least two peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least three peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least four peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least five peaks selected from 5.5±0.2, 11.1±0.2, 14.3±0.2, 15.9±0.2, 16.7±0.2, 17.0±0.2, 17.5±0.2, 19.1±0.2, 24.4±0.2 and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least six peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least seven peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least eight peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having at least nine peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having one peak selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having two peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having three peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having four peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having five peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having six peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having seven peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having eight peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having nine peaks selected from 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having a peak at 5.5±0.2, 11.1±0.2, 14.3±0.2, 15.9±0.2, 16.7±0.2, 17.0±0.2, 17.5±0.2, 19.1±0.2, 24.4±0.2, and 24.9±0.2 °2θ (e.g., 5.5±0.1, 11.1±0.1, 14.3±0.1, 15.9±0.1, 16.7±0.1, 17.0±0.1, 17.5±0.1, 19.1±0.1, 24.4±0.1, and 24.9±0.1 °2θ) using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, from about 24.2 to about 24.6, and from about 24.7 to about 25.1 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, from about 18.9 to about 19.3, from about 24.2 to about 24.6, and from about 24.7 to about 25.1 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, from about 17.3 to about 17.7, from about 18.9 to about 19.3, from about 24.2 to about 24.6, and from about 24.7 to about 25.1 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, from about 16.8 to about 17.2, from about 17.3 to about 17.7, from about 18.9 to about 19.3, from about 24.2 to about 24.6, and from about 24.7 to about 25.1 °2θ using Cu Kα radiation.

In some embodiments, the compound (e.g., the crystalline form of the benzoate salt of Compound 5R) is characterized by an XRPD pattern having a peak at from about 5.3 to about 5.7, from about 10.9 to about 11.3, from about 14.1 to about 14.5, from about 15.7 to about 16.1, from about 16.5 to about 16.9, from about 16.8 to about 17.2, from about 17.3 to about 17.7, from about 18.9 to about 19.3, from about 24.2 to about 24.6, and from about 24.7 to about 25.1 °2θ using Cu Kα radiation.

In some embodiments, the compound (e.g., the crystalline form of the benzoate salt of Compound 5R) is characterized by an XRPD pattern having a peak at from about 5.4 to about 5.6, from about 11.0 to about 11.2, from about 14.2 to about 14.4, from about 15.8 to about 16.0, from about 16.6 to about 16.8, from about 16.9 to about 17.1, from about 17.4 to about 17.6, from about 19.0 to about 19.2, from about 24.3 to about 24.5, and from about 24.8 to about 25.0 °2θ using Cu Kα radiation.

In some embodiments, the compound (e.g., the crystalline form of the benzoate salt of Compound 5R) is characterized by an XRPD pattern having a peak at about 5.51, about 11.10, about 14.33, about 15.93, about 16.74, about 17.04, about 17.45, about 19.14, about 24.44, and about 24.86 °2θ using Cu Kα radiation.

### Compound 5R Benzoate Salt Type E

In some embodiments, the compound is a benzoate salt of Compound 5R.

In some embodiments, the compound is a crystalline form of a benzoate salt of Compound 5R.

In some embodiments, the compound (e.g., the crystalline form of the benzoate salt of Compound 5R) is characterized by an XRPD pattern having at least one peak selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having at least two peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having at least three peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having at least four peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having one peak selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having two peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having three peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having four peaks selected from 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having a peak at 5.7±0.2, 6.2±0.2, 12.6±0.2, 15.4±0.2, and 25.1±0.2 °2θ (e.g., 5.7±0.1, 6.2±0.1, 12.6±0.1, 15.4±0.1, and 25.1±0.1 °2θ) using Cu Kα radiation.

In some embodiments, the compound (e.g., the crystalline form of the benzoate salt of Compound 5R) is characterized by an XRPD pattern having a peak at from about 5.5 to about 5.9, from about 6.1 to about 6.5, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 5.5 to about 5.9, from about 6.1 to about 6.5, from about 12.4 to about 12.8, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 5.5 to about 5.9, from about 6.1 to about 6.5, from about 12.4 to about 12.8, from about 15.2 to about 15.6, and from about 24.9 to about 25.3 °2θ using Cu Kα radiation.

In some embodiments, the compound (e.g., the crystalline form of the benzoate salt of Compound 5R) is characterized by an XRPD pattern having a peak at from about 5.6 to about 5.8, from about 6.2 to about 6.4, from about 12.5 to about 12.7, from about 15.3 to about 15.5, and from about 25.0 to about 25.2 °2θ using Cu Kα radiation.

In some embodiments, the compound (e.g., the crystalline form of the benzoate salt of Compound 5R) is characterized by an XRPD pattern having a peak at about 5.69, about 6.25, about 12.57, about 15.36, and about 25.11 °2θ using Cu Kα radiation.

### Compound 5R Benzoate Salt Type F

In some embodiments, the compound is a benzoate salt of Compound 5R.

In some embodiments, the compound is a crystalline form of a benzoate salt of Compound 5R.

In some embodiments, the compound (e.g., the crystalline form of the benzoate salt of Compound 5R) is characterized by an XRPD pattern having at least one peak selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least two peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least three peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least four peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least five peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least six peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least seven peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having at least eight peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having one peak selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having two peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having three peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having four peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having five peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having six peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having seven peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having eight peaks selected from 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having a peak at 6.1±0.2, 12.3±0.2, 16.3±0.2, 18.3±0.2, 21.2±0.2, 22.2±0.2, 23.1±0.2, 24.4±0.2, and 26.3±0.2 °2θ (e.g., 6.1±0.1, 12.3±0.1, 16.3±0.1, 18.3±0.1, 21.2±0.1, 22.2±0.1, 23.1±0.1, 24.4±0.1, and 26.3±0.1 °2θ) using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, from about 18.1 to about 18.5, and from about 24.2 to about 24.6 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, from about 18.1 to about 18.5, from about 24.2 to about 24.6, and from about 26.1 to about 26.5 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, from about 18.1 to about 18.5, from about 21.0 to about 21.4, from about 24.2 to about 24.6, and from about 26.1 to about 26.5 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, from about 18.1 to about 18.5, from about 21.0 to about 21.4, from about 22.9 to about 23.3, from about 24.2 to about 24.6, and from about 26.1 to about 26.5 °2θ using Cu Kα radiation.

In some embodiments, the compound (e.g., the crystalline form of the benzoate salt of Compound 5R) is characterized by an XRPD pattern having a peak at from about 5.9 to about 6.3, from about 12.1 to about 12.5, from about 16.1 to about 16.5, from about 18.1 to about 18.5, from about 21.0 to about 21.4, from about 22.0 to about 22.4, from about 22.9 to about 23.3, from about 24.2 to about 24.6, and from about 26.1 to about 26.5 °2θ using Cu Kα radiation.

In some embodiments, the compound (e.g., the crystalline form of the benzoate salt of Compound 5R) is characterized by an XRPD pattern having a peak at from about 6.0 to about 6.2, from about 12.2 to about 12.4, from about 16.2 to about 16.4, from about 18.2 to about 18.4, from about 21.1 to about 21.3, from about 22.1 to about 22.3, from about 23.0 to about 23.2, from about 24.3 to about 24.5, and from about 26.2 to about 26.4 °2θ using Cu Kα radiation.

In some embodiments, the compound (e.g., the crystalline form of the benzoate salt of Compound 5R) is characterized by an XRPD pattern having a peak at about 6.08, about 12.29, about 16.27, about 18.34, about 21.22, about 22.16, about 23.10, about 24.41, and about 26.25 °2θ using Cu Kα radiation.

In some embodiments, the compound is Compound 5S, a tautomer thereof, a pharmaceutically acceptable salt thereof, or a pharmaceutically acceptable salt of the tautomer.

In some embodiments, the compound is Compound 5S.

In some embodiments, the compound is a crystalline form of Compound 5S.

In some embodiments, the compound is a pharmaceutically acceptable salt of Compound 5S.

In some embodiments, the compound is a crystalline form of a pharmaceutically acceptable salt of Compound 5S.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 5S.

In some embodiments, the compound is a hydrochloride salt of Compound 5S.

In some embodiments, the compound is a crystalline form of a hydrochloride salt of Compound 5S.

### Compound 6

In some embodiments, the compound is

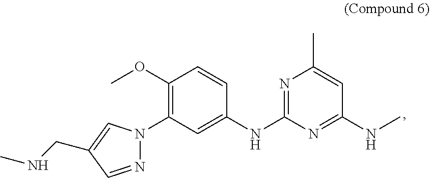

a tautomer thereof, a pharmaceutically acceptable salt thereof, or a pharmaceutically acceptable salt of the tautomer.

In some embodiments, the compound is Compound 6.

In some embodiments, the compound is a crystalline form of Compound 6.

In some embodiments, the crystalline form of Compound 6 is an anhydrate.

In some embodiments, the compound is a pharmaceutically acceptable salt of Compound 6.

In some embodiments, the compound is a crystalline form of a pharmaceutically acceptable salt of Compound 6.

In some embodiments, the crystalline form of the pharmaceutically acceptable salt of Compound 6 is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 6.

### Compound 6 Freebase Type A

In some embodiments, the compound is Compound 6.

In some embodiments, the compound is a crystalline form of Compound 6.

In some embodiments, the compound (e.g., the crystalline form of Compound 4R) is characterized by an XRPD pattern having at least one peak selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least two peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least three peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least four peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least five peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least six peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having at least seven peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having one peak selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having two peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having three peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having four peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having five peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having six peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having seven peaks selected from 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having a peak at 4.5±0.2, 9.7±0.2, 10.5±0.2, 13.5±0.2, 15.3±0.2, 18.1±0.2, 24.3±0.2, and 25.8±0.2 °2θ (e.g., 4.5±0.1, 9.7±0.1, 10.5±0.1, 13.5±0.1, 15.3±0.1, 18.1±0.1, 24.3±0.1, and 25.8±0.1 °2θ) using Cu Kα radiation.

In some embodiments, the compound (e.g., the crystalline form of Compound 4R) is characterized by an XRPD pattern having a peak at from about 4.3 to about 4.7, from about 13.3 to about 13.7, and from about 25.6 to about 26.0 °2θ using Cu Kα radiation.

In some embodiments, the compound (e.g., the crystalline form of Compound 4R) is characterized by an XRPD pattern having a peak at from about 4.3 to about 4.7, from about 10.3 to about 10.7, from about 13.3 to about 13.7, and from about 25.6 to about 26.0 °2θ using Cu Kα radiation.

In some embodiments, the compound (e.g., the crystalline form of Compound 4R) is characterized by an XRPD pattern having a peak at from about 4.3 to about 4.7, from about 9.5 to about 9.9, from about 10.3 to about 10.7, from about 13.3 to about 13.7, and from about 25.6 to about 26.0 °2θ using Cu Kα radiation.

In some embodiments, the compound (e.g., the crystalline form of Compound 4R) is characterized by an XRPD pattern having a peak at from about 4.3 to about 4.7, from about 9.5 to about 9.9, from about 10.3 to about 10.7, from about 13.3 to about 13.7, from about 17.9 to about 18.3, and from about 25.6 to about 26.0 °2θ using Cu Kα radiation.

In some embodiments, the compound (e.g., the crystalline form of Compound 4R) is characterized by an XRPD pattern having a peak at from about 4.3 to about 4.7, from about 9.5 to about 9.9, from about 10.3 to about 10.7, from about 13.3 to about 13.7, from about 15.1 to about 15.5, from about 17.9 to about 18.3, and from about 25.6 to about 26.0 °2θ using Cu Kα radiation.

In some embodiments, the compound (e.g., the crystalline form of Compound 4R) is characterized by an XRPD pattern having a peak at from about 4.3 to about 4.7, from about 9.5 to about 9.9, from about 10.3 to about 10.7, from about 13.3 to about 13.7, from about 15.1 to about 15.5, from about 17.9 to about 18.3, from about 24.1 to about 24.5, and from about 25.6 to about 26.0 °2θ using Cu Kα radiation.

In some embodiments, the compound (e.g., the crystalline form of Compound 4R) is characterized by an XRPD pattern having a peak at from about 4.4 to about 4.6, from about 9.6 to about 9.8, from about 10.4 to about 10.6, from about 13.4 to about 13.6, from about 15.2 to about 15.4, from about 18.0 to about 18.2, from about 24.2 to about 24.4, and from about 25.7 to about 25.9 °2θ using Cu Kao radiation.

In some embodiments, the compound (e.g., the crystalline form of Compound 4R) is characterized by an XRPD pattern having a peak at about 4.50, about 9.67, about 10.47, about 13.49, about 15.31, about 18.05, about 24.33, and about 25.77 °2θ using Cu Kα radiation.

In some embodiments, the compound (e.g., the crystalline form of Compound 6) has an endothermic peak top temperature in differential scanning calorimeter (DSC) analysis at between about 175° C. and about 215° C., between about 180° C. and about 210° C., between about 185° C. and about 205° C., between about 190° C. and about 200° C., or between about 192° C. and about 195° C.

In some embodiments, the compound (e.g., the crystalline form of Compound 6) has an endothermic peak top temperature in differential scanning calorimeter (DSC) analysis at about 200° C. and about 240° C., between about 205° C. and about 235° C., between about 210° C. and about 230° C., between about 214° C. and about 225° C., or between about 216° C. and about 219° C.

In some embodiments, the compound (e.g., the crystalline form of Compound 6) has an endothermic peak top temperature in differential scanning calorimeter (DSC) analysis at about 193.6° C. and/or at about 217.6° C.

### Compound 6 Hydrochloride Salt Type A

In some embodiments, the compound is a hydrochloride salt of Compound 6.

In some embodiments, the compound is a crystalline form of a hydrochloride salt of Compound 6.

In some embodiments, the compound (e.g., the crystalline form of the hydrochloride salt of Compound 6) is characterized by an XRPD pattern having at least one peak selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least two peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least three peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least four peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least five peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least six peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least seven peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least eight peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having at least nine peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having one peak selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having two peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having three peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having four peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having five peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having six peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having seven peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having eight peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having nine peaks selected from 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having a peak at 5.3±0.2, 9.9±0.2, 10.8±0.2, 11.5±0.2, 19.7±0.2, 21.5±0.2, 24.1±0.2, 25.1±0.2, 27.1±0.2, and 27.6±0.2 °2θ (e.g., 5.3±0.1, 9.9±0.1, 10.8±0.1, 11.5±0.1, 19.7±0.1, 21.5±0.1, 24.1±0.1, 25.1±0.1, 27.1±0.1, and 27.6±0.1 °2θ) using Cu Kα radiation.

In some embodiments, the compound (e.g., the crystalline form of the hydrochloride salt of Compound 6) is characterized by an XRPD pattern having a peak at from about 9.7 to about 10.1, from about 10.6 to about 11.0, and from about 24.9 to about 25.3 °2θ using Cu Kα radiation.

In some embodiments, the compound (e.g., the crystalline form of the hydrochloride salt of Compound 6) is characterized by an XRPD pattern having a peak at from about 9.7 to about 10.1, from about 10.6 to about 11.0, from about 24.9 to about 25.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 9.7 to about 10.1, from about 10.6 to about 11.0, from about 24.9 to about 25.3, from about 26.9 to about 27.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 9.7 to about 10.1, from about 10.6 to about 11.0, from about 11.3 to about 11.7, from about 24.9 to about 25.3, from about 26.9 to about 27.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 9.7 to about 10.1, from about 10.6 to about 11.0, from about 11.3 to about 11.7, from about 23.9 to about 24.3, from about 24.9 to about 25.3, from about 26.9 to about 27.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 5.0 to about 5.4, from about 9.7 to about 10.1, from about 10.6 to about 11.0, from about 11.3 to about 11.7, from about 23.9 to about 24.3, from about 24.9 to about 25.3, from about 26.9 to about 27.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 5.0 to about 5.4, from about 9.7 to about 10.1, from about 10.6 to about 11.0, from about 11.3 to about 11.7, from about 21.3 to about 21.7, from about 23.9 to about 24.3, from about 24.9 to about 25.3, from about 26.9 to about 27.3, and from about 27.4 to about 27.8 °2θ using Cu Kao radiation.

In some embodiments, the compound (e.g., the crystalline form of the hydrochloride salt of Compound 6) is characterized by an XRPD pattern having a peak at from about 5.0 to about 5.4, from about 9.7 to about 10.1, from about 10.6 to about 11.0, from about 11.3 to about 11.7, from about 19.5 to about 19.9, from about 21.3 to about 21.7, from about 23.9 to about 24.3, from about 24.9 to about 25.3, from about 26.9 to about 27.3, and from about 27.4 to about 27.8 °2θ using Cu Kα radiation.

In some embodiments, the compound (e.g., the crystalline form of the hydrochloride salt of Compound 6) is characterized by an XRPD pattern having a peak at from about 5.1 to about 5.3, from about 9.8 to about 10.0, from about 10.7 to about 10.9, from about 11.4 to about 11.6, from about 19.6 to about 19.8, from about 21.4 to about 21.6, from about 24.0 to about 24.2, from about 25.0 to about 25.2, from about 27.0 to about 27.2, and from about 27.5 to about 27.7 °2θ using Cu Kα radiation.

In some embodiments, the compound (e.g., the crystalline form of the hydrochloride salt of Compound 6) is characterized by an XRPD pattern having a peak at about 5.24, about 9.85, about 10.75, about 11.48, about 19.67, about 21.48, about 24.09, about 25.12, about 27.05, and about 27.62 °2θ using Cu Kα radiation.

In some embodiments, the compound (e.g., the crystalline form of the hydrochloride salt of Compound 6) has an endothermic peak top temperature in differential scanning calorimeter (DSC) analysis at between about 140° C. and about 180° C., between about 145° C. and about 175° C., between about 150° C. and about 170° C., between about 155° C. and about 165° C., or between about 159° C. and about 160° C.

In some embodiments, the compound (e.g., the crystalline form of the hydrochloride salt of Compound 6) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 207° C. and about 208° C.

In some embodiments, the compound (e.g., the crystalline form of the hydrochloride salt of Compound 6) has an endothermic peak top temperature in differential scanning calorimeter (DSC) analysis at between about 195° C. and about 235° C., between about 200° C. and about 230° C., between about 205° C. and about 225° C., between about 210° C. and about 220° C., or between about 216° C. and about 218° C.

In some embodiments, the compound (e.g., the crystalline form of the hydrochloride salt of Compound 6) has an endothermic peak top temperature in differential scanning calorimeter (DSC) analysis at between about 260° C. and about 300° C., between about 265° C. and about 295° C., between about 270° C. and about 290° C., between about 275° C. and about 285° C., or between about 277° C. and about 279° C.

In some embodiments, the compound (e.g., the crystalline form of the hydrochloride salt of Compound 6) has an endothermic peak top temperature in differential scanning calorimeter (DSC) analysis at about 159.5° C., at about 207.3° C., at about 216.9° C., and/or at about 278.1° C.

### Compound 6 Glycolate Salt Type A

In some embodiments, the compound is a glycolate salt of Compound 6.

In some embodiments, the compound is a crystalline form of a glycolate salt of Compound 6.

In some embodiments, the compound (e.g., the crystalline form of the glycolate salt of Compound 6) is characterized by an XRPD pattern having at least one peak selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least two peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least three peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least four peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least five peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least six peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having at least seven peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having one peak selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having two peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having three peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having four peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kao radiation.

In some embodiments, the compound (e.g., the crystalline form of the glycolate salt of Compound 6) is characterized by an XRPD pattern having five peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having six peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having seven peaks selected from 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kα radiation.

In some embodiments, the compound (e.g., the crystalline form of the glycolate salt of Compound 6) is characterized by an XRPD pattern having a peak at 5.7±0.2, 7.0±0.2, 10.3±0.2, 15.1±0.2, 16.1±0.2, 21.6±0.2, 25.8±0.2, and 27.7±0.2 °2θ (e.g., 5.7±0.1, 7.0±0.1, 10.3±0.1, 15.1±0.1, 16.1±0.1, 21.6±0.1, 25.8±0.1, and 27.7±0.1 °2θ) using Cu Kao radiation.

In some embodiments, the compound (e.g., the crystalline form of the glycolate salt of Compound 6) is characterized by an XRPD pattern having a peak at from about 5.5 to about 5.9, from about 6.8 to about 7.2, and from about 25.6 to about 26.0 °2θ using Cu Kα radiation.

In some embodiments, the compound (e.g., the crystalline form of the glycolate salt of Compound 6) is characterized by an XRPD pattern having a peak at from about 5.5 to about 5.9, from about 6.8 to about 7.2, from about 25.6 to about 26.0, from about 27.5 to about 27.9 °2θ using Cu Kα radiation.

In some embodiments, the compound (e.g., the crystalline form of the glycolate salt of Compound 6) is characterized by an XRPD pattern having a peak at from about 5.5 to about 5.9, from about 6.8 to about 7.2, from about 10.1 to about 10.5, from about 25.6 to about 26.0, from about 27.5 to about 27.9 °2θ using Cu Kα radiation.

In some embodiments, the compound (e.g., the crystalline form of the glycolate salt of Compound 6) is characterized by an XRPD pattern having a peak at from about 5.5 to about 5.9, from about 6.8 to about 7.2, from about 10.1 to about 10.5, from about 21.4 to about 21.8, from about 25.6 to about 26.0, and from about 27.5 to about 27.9 °2θ using Cu Kα radiation.

In some embodiments, the compound (e.g., the crystalline form of the glycolate salt of Compound 6) is characterized by an XRPD pattern having a peak at from about 5.5 to about 5.9, from about 6.8 to about 7.2, from about 10.1 to about 10.5, from about 14.9 to about 15.3, from about 21.4 to about 21.8, from about 25.6 to about 26.0, and from about 27.5 to about 27.9 °2θ using Cu Kα radiation.

In some embodiments, the compound (e.g., the crystalline form of the glycolate salt of Compound 6) is characterized by an XRPD pattern having a peak at from about 5.5 to about 5.9, from about 6.8 to about 7.2, from about 10.1 to about 10.5, from about 14.9 to about 15.3, from about 15.9 to about 16.3, from about 21.4 to about 21.8, from about 25.6 to about 26.0, and from about 27.5 to about 27.9 °2θ using Cu Kα radiation.

In some embodiments, the compound (e.g., the crystalline form of the glycolate salt of Compound 6) is characterized by an XRPD pattern having a peak at from about 5.6 to about 5.8, from about 6.9 to about 7.1, from about 10.2 to about 10.4, from about 15.0 to about 15.2, from about 16.0 to about 16.2, from about 21.5 to about 21.7, from about 25.7 to about 25.9, and from about 27.6 to about 27.8 °2θ using Cu Kα radiation.

In some embodiments, the compound (e.g., the crystalline form of the glycolate salt of Compound 6) is characterized by an XRPD pattern having a peak at about 5.71, about 7.04, about 10.25, about 15.12, about 16.07, about 21.64, about 25.79, and about 27.68 °2θ using Cu Kα radiation.

### Compound 6 Adipate Salt Type A

In some embodiments, the compound is an adipate salt of Compound 6.

In some embodiments, the compound is a crystalline form of an adipate salt of Compound 6.

In some embodiments, the compound (e.g., the crystalline form of the adipate salt of Compound 6) is characterized by an XRPD pattern having at least one peak selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least two peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least three peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least four peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least five peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least six peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having at least seven peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having one peak selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having two peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having three peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having four peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having five peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having six peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having seven peaks selected from 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having a peak at 5.8±0.2, 7.8±0.2, 10.5±0.2, 11.3±0.2, 14.4±0.2, 24.6±0.2, 25.6±0.2, and 26.3±0.2 °2θ (e.g., 5.8±0.1, 7.8±0.1, 10.5±0.1, 11.3±0.1, 14.4±0.1, 24.6±0.1, 25.6±0.1, and 26.3±0.1 °2θ) using Cu Kα radiation.

In some embodiments, the compound (e.g., the crystalline form of the adipate salt of Compound 6) is characterized by an XRPD pattern having a peak at from about 7.6 to about 8.0, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 7.6 to about 8.0, from about 11.1 to about 11.5, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 7.6 to about 8.0, from about 10.3 to about 10.7, from about 11.1 to about 11.5, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 7.6 to about 8.0, from about 10.3 to about 10.7, from about 11.1 to about 11.5, from about 14.2 to about 14.6, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 5.6 to about 6.0, from about 7.6 to about 8.0, from about 10.3 to about 10.7, from about 11.1 to about 11.5, from about 14.2 to about 14.6, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 5.6 to about 6.0, from about 7.6 to about 8.0, from about 10.3 to about 10.7, from about 11.1 to about 11.5, from about 14.2 to about 14.6, from about 24.4 to about 24.8, from about 25.4 to about 25.8, and from about 26.1 to about 26.5 °2θ using Cu Kα radiation.

In some embodiments, the compound (e.g., the crystalline form of the adipate salt of Compound 6) is characterized by an XRPD pattern having a peak at from about 5.7 to about 5.9, from about 7.7 to about 7.9, from about 10.4 to about 10.6, from about 11.2 to about 11.4, from about 14.3 to about 14.5, from about 24.5 to about 24.7, from about 25.5 to about 25.7, and from about 26.2 to about 26.4 °2θ using Cu Kα radiation.

In some embodiments, the compound (e.g., the crystalline form of the adipate salt of Compound 6) is characterized by an XRPD pattern having a peak at about 5.82, about 7.76, about 10.51, about 11.26, about 14.35, about 24.63, about 25.59, and about 26.28 °2θ using Cu Kα radiation.

In some embodiments, the compound (e.g., the crystalline form of the adipate salt of Compound 6) has an endothermic peak top temperature in differential scanning calorimeter (DSC) analysis at between about 75° C. and about 115° C., between about 80° C. and about 110° C., between about 85° C. and about 105° C., between about 90° C. and about 100° C., or between about 96° C. and about 97° C.

In some embodiments, the compound (e.g., the crystalline form of the adipate salt of Compound 6) has an endothermic peak top temperature in differential scanning calorimeter (DSC) analysis at between about 150° C. and about 190° C., between about 155° C. and about 185° C., between about 160° C. and about 180° C., between about 165° C. and about 175° C., or between about 171° C. and about 173° C.

In some embodiments, the compound (e.g., the crystalline form of the adipate salt of Compound 6) has an endothermic peak top temperature in differential scanning calorimeter (DSC) analysis at between about 175° C. and about 215° C., between about 180° C. and about 210° C., between about 185° C. and about 205° C., between about 190° C. and about 200° C., or between about 194° C. and about 196° C.

In some embodiments, the compound (e.g., the crystalline form of the adipate salt of Compound 6) has an endothermic peak top temperature in differential scanning calorimeter (DSC) analysis at about 96.5° C., at about 172.2° C., and/or at about 195.2° C.

### Compound 6 Adipate Salt Type B

In some embodiments, the compound is an adipate salt of Compound 6.

In some embodiments, the compound is a crystalline form of an adipate salt of Compound 6.

In some embodiments, the compound (e.g., the crystalline form of the adipate salt of Compound 6) is characterized by an XRPD pattern having at least one peak selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least two peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least three peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least four peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least five peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least six peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least seven peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least eight peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having at least nine peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having one peak selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having two peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having three peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having four peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having five peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having six peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having seven peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having eight peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having nine peaks selected from 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having a peak at 5.3±0.2, 6.0±0.2, 8.1±0.2, 11.6±0.2, 11.9±0.2, 14.7±0.2, 21.6±0.2, 24.0±0.2, 25.5±0.2, and 26.4±0.2 °2θ (e.g., 5.3±0.1, 6.0±0.1, 8.1±0.1, 11.6±0.1, 11.9±0.1, 14.7±0.1, 21.6±0.1, 24.0±0.1, 25.5±0.1, and 26.4±0.1 °2θ) using Cu Kα radiation.

In some embodiments, the compound (e.g., the crystalline form of the adipate salt of Compound 6) is characterized by an XRPD pattern having a peak at from about 8.0 to about 8.2, from about 11.5 to about 11.7, and from about 25.4 to about 25.6 °2θ using Cu Kα radiation.

In some embodiments, the compound (e.g., the crystalline form of the adipate salt of Compound 6) is characterized by an XRPD pattern having a peak at from about 8.0 to about 8.2, from about 11.5 to about 11.7, from about 11.8 to about 12.0, and from about 25.4 to about 25.6 °2θ using Cu Kα radiation.

In some embodiments, the compound (e.g., the crystalline form of the adipate salt of Compound 6) is characterized by an XRPD pattern having a peak at from about 5.9 to about 6.1, from about 8.0 to about 8.2, from about 11.5 to about 11.7, from about 11.8 to about 12.0, and from about 25.4 to about 25.6 °2θ using Cu Kα radiation.

In some embodiments, the compound (e.g., the crystalline form of the adipate salt of Compound 6) is characterized by an XRPD pattern having a peak at from about 5.2 to about 5.3, from about 5.9 to about 6.1, from about 8.0 to about 8.2, from about 11.5 to about 11.7, from about 11.8 to about 12.0, and from about 25.4 to about 25.6 °2θ using Cu Kα radiation.

In some embodiments, the compound (e.g., the crystalline form of the adipate salt of Compound 6) is characterized by an XRPD pattern having a peak at from about 5.2 to about 5.3, from about 5.9 to about 6.1, from about 8.0 to about 8.2, from about 11.5 to about 11.7, from about 11.8 to about 12.0, from about 23.9 to about 24.1, and from about 25.4 to about 25.6 °2θ using Cu Kα radiation.

In some embodiments, the compound (e.g., the crystalline form of the adipate salt of Compound 6) is characterized by an XRPD pattern having a peak at from about 5.2 to about 5.3, from about 5.9 to about 6.1, from about 8.0 to about 8.2, from about 11.5 to about 11.7, from about 11.8 to about 12.0, from about 23.9 to about 24.1, from about 25.4 to about 25.6, and from about 26.3 to about 26.5 °2θ using Cu Kα radiation.

In some embodiments, the compound (e.g., the crystalline form of the adipate salt of Compound 6) is characterized by an XRPD pattern having a peak at from about 5.2 to about 5.3, from about 5.9 to about 6.1, from about 8.0 to about 8.2, from about 11.5 to about 11.7, from about 11.8 to about 12.0, from about 14.6 to about 14.8, from about 23.9 to about 24.1, from about 25.4 to about 25.6, and from about 26.3 to about 26.5 °2θ using Cu Kα radiation.

In some embodiments, the compound (e.g., the crystalline form of the adipate salt of Compound 6) is characterized by an XRPD pattern having a peak at from about 5.2 to about 5.3, from about 5.9 to about 6.1, from about 8.0 to about 8.2, from about 11.5 to about 11.7, from about 11.8 to about 12.0, from about 14.6 to about 14.8, from about 21.5 to about 21.7, from about 23.9 to about 24.1, from about 25.4 to about 25.6, and from about 26.3 to about 26.5 °2θ using Cu Kα radiation.

In some embodiments, the compound (e.g., the crystalline form of the adipate salt of Compound 6) is characterized by an XRPD pattern having a peak at about 5.28, about 5.96, about 8.11, about 11.59, about 11.91, about 14.73, about 21.58, about 24.00, about 25.53, and about 26.36 °2θ using Cu Kα radiation.

In some embodiments, the compound (e.g., the crystalline form of the adipate salt of Compound 6) has an endothermic peak top temperature in differential scanning calorimeter (DSC) analysis at between about 140° C. and about 180° C., between about 145° C. and about 175° C., between about 150° C. and about 170° C., between about 155° C. and about 165° C., or between about 159° C. and about 160° C.

In some embodiments, the compound (e.g., the crystalline form of the adipate salt of Compound 6) has an endothermic peak top temperature in differential scanning calorimeter (DSC) analysis at between about 170° C. and about 210° C., between about 175° C. and about 205° C., between about 180° C. and about 200° C., between about 185° C. and about 195° C., or between about 191° C. and about 193° C.

In some embodiments, the compound (e.g., the crystalline form of the adipate salt of Compound 6) has an endothermic peak top temperature in differential scanning calorimeter (DSC) analysis at about 159.5° C. and/or at about 191.9° C.

### Compound 7

In some embodiments, the compound is

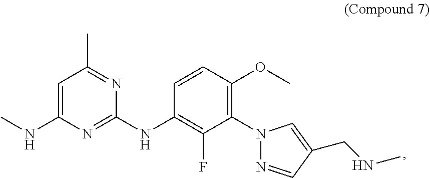

a tautomer thereof, a pharmaceutically acceptable salt thereof, or a pharmaceutically acceptable salt of the tautomer.

In some embodiments, the compound is Compound 7.

In some embodiments, the compound is a crystalline form of Compound 7.

In some embodiments, the crystalline form of Compound 7 is an anhydrate.

In some embodiments, the compound is a pharmaceutically acceptable salt of Compound 7.

In some embodiments, the compound is a crystalline form of a pharmaceutically acceptable salt of Compound 7.

In some embodiments, the crystalline form of the pharmaceutically acceptable salt of Compound 7 is an anhydrate.

In some embodiments, the compound is a hydrochloride salt, sulfate salt, glycolate salt, adipate salt, succinate salt, oxalate salt, phosphate salt, fumarate salt, hippurate salt, gentisate salt, or benzoate salt of Compound 7.

### Compound 7 Hydrochloride Salt Type A

In some embodiments, the compound is a hydrochloride salt of Compound 7.

In some embodiments, the compound is a crystalline form of a hydrochloride salt of Compound 7.

In some embodiments, the compound (e.g., the crystalline form of the hydrochloride salt of Compound 7) is characterized by an XRPD pattern having at least one peak selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least two peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least three peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least four peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least five peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least six peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least seven peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least eight peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having at least nine peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having one peak selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having two peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.51±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having three peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having four peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having five peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having six peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having seven peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having eight peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having nine peaks selected from 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having a peak at 6.8±0.2, 9.4±0.2, 12.1±0.2, 14.5±0.2, 15.0±0.2, 18.7±0.2, 24.2±0.2, 25.1±0.2, 25.6±0.2, and 26.8±0.2 °2θ (e.g., 6.8±0.1, 9.4±0.1, 12.1±0.1, 14.5±0.1, 15.0±0.1, 18.7±0.1, 24.2±0.1, 25.1±0.1, 25.6±0.1, and 26.8±0.1 °2θ) using Cu Kα radiation.

In some embodiments, the compound (e.g., the crystalline form of the hydrochloride salt of Compound 7) is characterized by an XRPD pattern having a peak at from about 6.6 to about 7.0, from about 24.9 to about 25.3, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 14.3 to about 14.7, from about 24.9 to about 25.3, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 14.3 to about 14.7, from about 14.8 to about 15.2, from about 24.9 to about 25.3, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 14.3 to about 14.7, from about 14.8 to about 15.2, from about 24.9 to about 25.3, from about 25.4 to about 25.8, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 14.3 to about 14.7, from about 14.8 to about 15.2, from about 24.0 to about 24.4, from about 24.9 to about 25.3, from about 25.4 to about 25.8, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 11.9 to about 12.3, from about 14.3 to about 14.7, from about 14.8 to about 15.2, from about 24.0 to about 24.4, from about 24.9 to about 25.3, from about 25.4 to about 25.8, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 11.9 to about 12.3, from about 14.3 to about 14.7, from about 14.8 to about 15.2, from about 18.5 to about 18.9, from about 24.0 to about 24.4, from about 24.9 to about 25.3, from about 25.4 to about 25.8, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.6 to about 7.0, from about 9.2 to about 9.6, from about 11.9 to about 12.3, from about 14.3 to about 14.7, from about 14.8 to about 15.2, from about 18.5 to about 18.9, from about 24.0 to about 24.4, from about 24.9 to about 25.3, from about 25.4 to about 25.8, and from about 26.6 to about 27.0 °2θ using Cu Kα radiation.

In some embodiments, the compound (e.g., the crystalline form of the hydrochloride salt of Compound 7) is characterized by an XRPD pattern having a peak at from about 6.7 to about 6.9, from about 9.3 to about 9.5, from about 12.0 to about 12.2, from about 14.4 to about 14.6, from about 14.9 to about 15.1, from about 18.6 to about 18.8, from about 24.1 to about 24.3, from about 25.0 to about 25.2, from about 25.5 to about 25.7, and from about 26.7 to about 26.9 °2θ using Cu Kα radiation.

In some embodiments, the compound (e.g., the crystalline form of the hydrochloride salt of Compound 7) is characterized by an XRPD pattern having a peak at about 6.8, about 9.4, about 12.1, about 14.5, about 15.0, about 18.7, about 24.2, about 25.1, about 25.6, and about 26.8 °2θ using Cu Kα radiation.

In some embodiments, the compound (e.g., the crystalline form of the hydrochloride salt of Compound 7) has an endothermic peak top temperature in differential scanning calorimeter (DSC) analysis at between about 55° C. and about 95° C., between about 60° C. and about 90° C., between about 65° C. and about 85° C., between about 70° C. and about 80° C., or between about 76° C. and about 78° C.

In some embodiments, the compound (e.g., the crystalline form of the hydrochloride salt of Compound 7) has an endothermic peak top temperature in differential scanning calorimeter (DSC) analysis at between about 110° C. and about 150° C., between about 115° C. and about 145° C., between about 120° C. and about 140° C., between about 125° C. and about 135° C., or between about 127° C. and about 129° C.

In some embodiments, the compound (e.g., the crystalline form of the hydrochloride salt of Compound 7) has an endothermic peak top temperature in differential scanning calorimeter (DSC) analysis at between about 150° C. and about 190° C., between about 155° C. and about 185° C., between about 160° C. and about 180° C., between about 165° C. and about 175° C., or between about 169° C. and about 171° C.

In some embodiments, the compound (e.g., the crystalline form of the hydrochloride salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 209° C. and about 211° C.

In some embodiments, the compound (e.g., the crystalline form of the hydrochloride salt of Compound 7) has an endothermic peak top temperature in differential scanning calorimeter (DSC) analysis at between about 210° C. and about 250° C., between about 215° C. and about 245° C., between about 220° C. and about 240° C., between about 225° C. and about 235° C., or between about 231° C. and about 233° C.

In some embodiments, the compound (e.g., the crystalline form of the hydrochloride salt of Compound 7) has an endothermic peak top temperature in differential scanning calorimeter (DSC) analysis at about 77.3° C., at about 128.2° C., at about 170.2° C., at about 210.6° C., and/or at about 231.7° C.

### Compound 7 Hydrochloride Salt Type B

In some embodiments, the compound is a hydrochloride salt of Compound 7.

In some embodiments, the compound is a crystalline form of a hydrochloride salt of Compound 7.

In some embodiments, the compound (e.g., the crystalline form of the hydrochloride salt of Compound 7) is characterized by an XRPD pattern having at least one peak selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having at least two peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having at least three peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having at least four peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having at least five peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having at least six peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having one peak selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kao radiation.

In some embodiments, the compound (e.g., the crystalline form of the hydrochloride salt of Compound 7) is characterized by an XRPD pattern having two peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having three peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having four peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having five peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having six peaks selected from 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having a peak at 5.9±0.2, 8.3±0.2, 10.0±0.2, 11.7±0.2, 21.9±0.2, 25.1±0.2, and 26.9±0.2 °2θ (e.g., from 5.9±0.1, 8.3±0.1, 10.0±0.1, 11.7±0.1, 21.9±0.1, 25.1±0.1, and 26.9±0.1 °2θ) using Cu Kα radiation.

In some embodiments, the compound (e.g., the crystalline form of the hydrochloride salt of Compound 7) is characterized by an XRPD pattern having a peak at from about 8.1 to about 8.5, from about 9.8 to about 10.2, and from about 24.9 to about 25.3 °2θ using Cu Kα radiation.

In some embodiments, the compound (e.g., the crystalline form of the hydrochloride salt of Compound 7) is characterized by an XRPD pattern having a peak at from about 5.7 to about 6.1, from about 8.1 to about 8.5, from about 9.8 to about 10.2, and from about 24.9 to about 25.3 °2θ using Cu Kα radiation.

In some embodiments, the compound (e.g., the crystalline form of the hydrochloride salt of Compound 7) is characterized by an XRPD pattern having a peak at from about 5.7 to about 6.1, from about 8.1 to about 8.5, from about 9.8 to about 10.2, from about 24.9 to about 25.3, and from about 26.7 to about 27.1 °2θ using Cu Kα radiation.

In some embodiments, the compound (e.g., the crystalline form of the hydrochloride salt of Compound 7) is characterized by an XRPD pattern having a peak at from about 5.7 to about 6.1, from about 8.1 to about 8.5, from about 9.8 to about 10.2, from about 21.7 to about 22.1, from about 24.9 to about 25.3, and from about 26.7 to about 27.1 °2θ using Cu Kα radiation.

In some embodiments, the compound (e.g., the crystalline form of the hydrochloride salt of Compound 7) is characterized by an XRPD pattern having a peak at from about 5.7 to about 6.1, from about 8.1 to about 8.5, from about 9.8 to about 10.2, from about 11.5 to about 11.9, from about 21.7 to about 22.1, from about 24.9 to about 25.3, and from about 26.7 to about 27.1 °2θ using Cu Kα radiation.

In some embodiments, the compound (e.g., the crystalline form of the hydrochloride salt of Compound 7) is characterized by an XRPD pattern having a peak at from about 5.8 to about 6.0, from about 8.2 to about 8.4, from about 9.9 to about 10.1, from about 11.6 to about 11.8, from about 21.8 to about 22.0, from about 25.0 to about 25.2, and from about 26.8 to about 27.0 °2θ using Cu Kα radiation.

In some embodiments, the compound (e.g., the crystalline form of the hydrochloride salt of Compound 7) is characterized by an XRPD pattern having a peak at about 5.9, about 8.3, about 10.0, about 11.7, about 21.9, about 25.1, and about 26.9 °2θ using Cu Kα radiation.

In some embodiments, the compound (e.g., the crystalline form of the hydrochloride salt of Compound 7) has an endothermic peak top temperature in differential scanning calorimeter (DSC) analysis at between about 70° C. and about 110° C., between about 75° C. and about 105° C., between about 80° C. and about 100° C., between about 85° C. and about 95° C., or between about 87° C. and about 89° C.

In some embodiments, the compound (e.g., the crystalline form of the hydrochloride salt of Compound 7) has an endothermic peak top temperature in differential scanning calorimeter (DSC) analysis at between about 100° C. and about 140° C., between about 105° C. and about 135° C., between about 110° C. and about 130° C., between about 115° C. and about 125° C., or between about 118° C. and about 120° C.

In some embodiments, the compound (e.g., the crystalline form of the hydrochloride salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 208° C. and about 210° C.

In some embodiments, the compound (e.g., the crystalline form of the hydrochloride salt of Compound 7) has an endothermic peak top temperature in differential scanning calorimeter (DSC) analysis at about 87.8° C., at about 118.6° C., and/or at about 208.7° C.

### Compound 7 Oxalate Salt Type A

In some embodiments, the compound is an oxalate salt of Compound 7.

In some embodiments, the compound is a crystalline form of an oxalate salt of Compound 7.

In some embodiments, the compound (e.g., the crystalline form of the oxalate salt of Compound 7) is characterized by an XRPD pattern having at least one peak selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having at least two peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having at least three peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having at least four peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having at least five peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having at least six peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having one peak selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having two peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having three peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having four peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having five peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having six peaks selected from 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having a peak at 4.5±0.2, 8.7±0.2, 9.1±0.2, 9.7±0.2, 13.8±0.2, 24.9±0.2, and 25.4±0.2 °2θ (e.g., 4.5±0.1, 8.7±0.1, 9.1±0.1, 9.7±0.1, 13.8±0.1, 24.9±0.1, and 25.4±0.1 °2θ) using Cu Kα radiation.

In some embodiments, the compound (e.g., the crystalline form of the oxalate salt of Compound 7) is characterized by an XRPD pattern having a peak at from about 4.3 to about 4.7, from about 8.5 to about 8.9, and from about 8.9 to about 9.3 °2θ using Cu Kα radiation.

In some embodiments, the compound (e.g., the crystalline form of the oxalate salt of Compound 7) is characterized by an XRPD pattern having a peak at from about 4.3 to about 4.7, from about 8.5 to about 8.9, from about 8.9 to about 9.3, and from about 13.6 to about 13.8 °2θ using Cu Kα radiation.

In some embodiments, the compound (e.g., the crystalline form of the oxalate salt of Compound 7) is characterized by an XRPD pattern having a peak at from about 4.3 to about 4.7, from about 8.5 to about 8.9, from about 8.9 to about 9.3, from about 13.6 to about 13.8, and from about 25.2 to about 25.6 °2θ using Cu Kα radiation.

In some embodiments, the compound (e.g., the crystalline form of the oxalate salt of Compound 7) is characterized by an XRPD pattern having a peak at from about 4.3 to about 4.7, from about 8.5 to about 8.9, from about 8.9 to about 9.3, from about 9.4 to about 9.9, from about 13.6 to about 13.8, and from about 25.2 to about 25.6 °2θ using Cu Kα radiation.

In some embodiments, the compound (e.g., the crystalline form of the oxalate salt of Compound 7) is characterized by an XRPD pattern having a peak at from about 4.3 to about 4.7, from about 8.5 to about 8.9, from about 8.9 to about 9.3, from about 9.4 to about 9.9, from about 13.6 to about 13.8, from about 24.7 to about 25.1, and from about 25.2 to about 25.6 °2θ using Cu Kα radiation.

In some embodiments, the compound (e.g., the crystalline form of the oxalate salt of Compound 7) is characterized by an XRPD pattern having a peak at from about 4.4 to about 4.6, from about 8.6 to about 8.8, from about 9.0 to about 9.2, from about 9.6 to about 9.8, from about 13.7 to about 13.9, from about 24.8 to about 25.0, and from about 25.3 to about 25.5 °2θ using Cu Kα radiation.

In some embodiments, the compound (e.g., the crystalline form of the oxalate salt of Compound 7) is characterized by an XRPD pattern having a peak at about 4.5, about 8.7, about 9.1, about 9.7, about 13.8, about 24.9, and about 25.4 °2θ using Cu Kα radiation.

In some embodiments, the compound (e.g., the crystalline form of the oxalate salt of Compound 7) has an endothermic peak top temperature in differential scanning calorimeter (DSC) analysis at between about 125° C. and about 165° C., between about 130° C. and about 160° C., between about 135° C. and about 155° C., between about 140° C. and about 150° C., or between about 143° C. and about 145° C.

In some embodiments, the compound (e.g., the crystalline form of the oxalate salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 210° C. and about 212° C.

In some embodiments, the compound (e.g., the crystalline form of the oxalate salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 208° C. and about 210° C.

In some embodiments, the compound (e.g., the crystalline form of the oxalate salt of Compound 7) has an endothermic peak top temperature in differential scanning calorimeter (DSC) analysis at about 144.2° C., at about 211.2° C., and/or at about 208.7° C.

### Compound 7 Sulfate Salt Type A

In some embodiments, the compound is a sulfate salt of Compound 7.

In some embodiments, the compound is a crystalline form of a sulfate salt of Compound 7.

In some embodiments, the compound (e.g., the crystalline form of the sulfate salt of Compound 7) is characterized by an XRPD pattern having at least one peak selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least two peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least three peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least four peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least five peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least six peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least seven peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least eight peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least nine peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having at least ten peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having one peak selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g. 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having two peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having three peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having four peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having five peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having six peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having seven peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g. 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having eight peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having nine peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having ten peaks selected from 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having a peak at 13.1±0.2, 15.8±0.2, 17.9±0.2, 18.0±0.2, 18.9±0.2, 19.2±0.2, 19.7±0.2, 23.8±0.2, 25.1±0.2, 25.7±0.2, and 26.4±0.2 °2θ (e.g, 13.1±0.1, 15.8±0.1, 17.9±0.1, 18.0±0.1, 18.9±0.1, 19.2±0.1, 19.7±0.1, 23.8±0.1, 25.1±0.1, 25.7±0.1, and 26.4±0.1 °2θ) using Cu Kα radiation.

In some embodiments, the compound (e.g., the crystalline form of the sulfate salt of Compound 7) is characterized by an XRPD pattern having a peak at from about 15.6 to about 16.0, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 19.0 to about 19.4, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 19.0 to about 19.4, from about 19.5 to about 19.9, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 17.8 to about 18.2, from about 19.0 to about 19.4, from about 19.5 to about 19.9, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 17.8 to about 18.2, from about 18.7 to about 19.1, from about 19.0 to about 19.4, from about 19.5 to about 19.9, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 17.8 to about 18.2, from about 18.7 to about 19.1, from about 19.0 to about 19.4, from about 19.5 to about 19.9, from about 23.6 to about 24.0, from about 24.9 to about 25.3, and from about 26.2 to about 26.6 °2θ using Cu Kα radiation.

In some embodiments, the compound (e.g., the crystalline form of the sulfate salt of Compound 7) is characterized by an XRPD pattern having a peak at from about 15.6 to about 16.0, form about 17.7 to about 18.1, from about 17.8 to about 18.2, from about 18.7 to about 19.1, from about 19.0 to about 19.4, from about 19.5 to about 19.9, from about 23.6 to about 24.0, from about 24.9 to about 25.3, from about 25.5 to about 25.9, and from about 26.2 to about 26.6 °2θ using Cu Kα radiation.

In some embodiments, the compound (e.g., the crystalline form of the sulfate salt of Compound 7) is characterized by an XRPD pattern having a peak at from about 12.9 to about 13.3, from about 15.6 to about 16.0, form about 17.7 to about 18.1, from about 17.8 to about 18.2, from about 18.7 to about 19.1, from about 19.0 to about 19.4, from about 19.5 to about 19.9, from about 23.6 to about 24.0, from about 24.9 to about 25.3, from about 25.5 to about 25.9, and from about 26.2 to about 26.6 °2θ using Cu Kα radiation.

In some embodiments, the compound (e.g., the crystalline form of the sulfate salt of Compound 7) is characterized by an XRPD pattern having a peak at from about 13.0 to about 13.2, from about 15.7 to about 15.9, form about 17.8 to about 18.0, from about 17.9 to about 18.1, from about 18.8 to about 19.0, from about 19.1 to about 19.3, from about 19.6 to about 19.8, from about 23.7 to about 23.9, from about 25.0 to about 25.2, from about 25.6 to about 25.8, and from about 26.3 to about 26.5 °2θ using Cu Kα radiation.

In some embodiments, the compound (e.g., the crystalline form of the sulfate salt of Compound 7) is characterized by an XRPD pattern having a peak at about 13.1, about 15.8, about 17.9, about 18.0, about 18.9, about 19.2, about 19.7, about 23.8, about 25.1, about 25.7, and about 26.4 °2θ using Cu Kα radiation.

In some embodiments, the compound (e.g., the crystalline form of the sulfate salt of Compound 7) has an endothermic peak top temperature in differential scanning calorimeter (DSC) analysis at between about 95° C. and about 135° C., between about 100° C. and about 130° C., between about 105° C. and about 125° C., between about 110° C. and about 120° C., or between about 113° C. and about 115° C.

In some embodiments, the compound (e.g., the crystalline form of the sulfate salt of Compound 7) has an endothermic peak top temperature in differential scanning calorimeter (DSC) analysis at between about 130° C. and about 170° C., between about 135° C. and about 165° C., between about 140° C. and about 160° C., between about 145° C. and about 155° C., or between about 151° C. and about 153° C.

In some embodiments, the compound (e.g., the crystalline form of the sulfate salt of Compound 7) has an endothermic peak top temperature in differential scanning calorimeter (DSC) analysis at between about 165° C. and about 205° C., between about 170° C. and about 200° C., between about 175° C. and about 195° C., between about 180° C. and about 190° C., or between about 184° C. and about 186° C.

In some embodiments, the compound (e.g., the crystalline form of the sulfate salt of Compound 7) has an endothermic peak top temperature in differential scanning calorimeter (DSC) analysis at about 113.4° C., at about 152.1° C., and/or at about 185.3° C.

### Compound 7 Phosphate Salt Type A

In some embodiments, the compound is a phosphate salt of Compound 7.

In some embodiments, the compound is a crystalline form of a phosphate salt of Compound 7.

In some embodiments, the compound (e.g., the crystalline form of the phosphate salt of Compound 7) is characterized by an XRPD pattern having at least one peak selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having at least two peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having at least three peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having at least four peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having at least five peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having one peak selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having two peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having three peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having four peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having five peaks selected from 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having a peak at 13.8±0.2, 14.4±0.2, 15.3±0.2, 16.8±0.2, 24.1±0.2, and 25.0±0.2 °2θ (e.g., 13.8±0.1, 14.4±0.1, 15.3±0.1, 16.8±0.1, 24.1±0.1, and 25.0±0.1 °2θ) using Cu Kα radiation.

In some embodiments, the compound (e.g., the crystalline form of the phosphate salt of Compound 7) is characterized by an XRPD pattern having a peak at from about 14.2 to about 14.6, from about 23.9 to about 24.3, and from about 24.8 to about 25.2 °2θ using Cu Kα radiation.

In some embodiments, the compound (e.g., the crystalline form of the phosphate salt of Compound 7) is characterized by an XRPD pattern having a peak at from about 13.6 to about 14.0, from about 14.2 to about 14.6, from about 23.9 to about 24.3, and from about 24.8 to about 25.2 °2θ using Cu Kα radiation.

In some embodiments, the compound (e.g., the crystalline form of the phosphate salt of Compound 7) is characterized by an XRPD pattern having a peak at from about 13.6 to about 14.0, from about 14.2 to about 14.6, from about 15.1 to about 15.5, from about 23.9 to about 24.3, and from about 24.8 to about 25.2 °2θ using Cu Kα radiation.

In some embodiments, the compound (e.g., the crystalline form of the phosphate salt of Compound 7) is characterized by an XRPD pattern having a peak at from about 13.6 to about 14.0, from about 14.2 to about 14.6, from about 15.1 to about 15.5, from about 16.6 to about 17.0, from about 23.9 to about 24.3, and from about 24.8 to about 25.2 °2θ using Cu Kα radiation.

In some embodiments, the compound (e.g., the crystalline form of the phosphate salt of Compound 7) is characterized by an XRPD pattern having a peak at from about 13.7 to about 13.9, from about 14.3 to about 14.5, from about 15.2 to about 15.4, from about 16.7 to about 16.9, from about 24.0 to about 24.2, and from about 24.9 to about 25.1 °2θ using Cu Kα radiation.

In some embodiments, the compound (e.g., the crystalline form of the phosphate salt of Compound 7) is characterized by an XRPD pattern having a peak at about 13.8, about 14.4, about 15.3, about 16.8, about 24.1, and about 25.0 °2θ using Cu Kα radiation.

In some embodiments, the compound (e.g., the crystalline form of the phosphate salt of Compound 7) has an endothermic peak top temperature in differential scanning calorimeter (DSC) analysis at between about 55° C. and about 95° C., between about 60° C. and about 90° C., between about 65° C. and about 85° C., between about 70° C. and about 80° C., or between about 76° C. and about 78° C.

In some embodiments, the compound (e.g., the crystalline form of the phosphate salt of Compound 7) has an endothermic peak top temperature in differential scanning calorimeter (DSC) analysis at between about 90° C. and about 130° C., between about 95° C. and about 125° C., between about 100° C. and about 120° C., between about 105° C. and about 115° C., or between about 109° C. and about 111° C.

In some embodiments, the compound (e.g., the crystalline form of the phosphate salt of Compound 7) has an endothermic peak top temperature in differential scanning calorimeter (DSC) analysis at between about 120° C. and about 160° C., between about 125° C. and about 155° C., between about 130° C. and about 150° C., between about 135° C. and about 145° C., or between about 139° C. and about 141° C.

In some embodiments, the compound (e.g., the crystalline form of the phosphate salt of Compound 7) has an endothermic peak top temperature in differential scanning calorimeter (DSC) analysis at between about 165° C. and about 205° C., between about 170° C. and about 200° C., between about 175° C. and about 195° C., between about 180° C. and about 190° C., or between about 183° C. and about 185° C.

In some embodiments, the compound (e.g., the crystalline form of the phosphate salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 209° C. and about 211° C.

In some embodiments, the compound (e.g., the crystalline form of the phosphate salt of Compound 7) has an endothermic peak top temperature in differential scanning calorimeter (DSC) analysis at about 76.7° C., at about 110.0° C., at about 140.3° C., at about 183.8° C., and/or at about 209.4° C.

### Compound 7 Fumarate Salt Type A

In some embodiments, the compound is a fumarate salt of Compound 7.

In some embodiments, the compound is a crystalline form of a fumarate salt of Compound 7.

In some embodiments, the compound (e.g., the crystalline form of the fumarate salt of Compound 7) is characterized by an XRPD pattern having at least one peak selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least two peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kao radiation.

In some embodiments, the compound (e.g., the crystalline form of the fumarate salt of Compound 7) is characterized by an XRPD pattern having at least three peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least four peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least five peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least six peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least seven peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having at least eight peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having one peak selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having two peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having three peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having four peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having five peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having six peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having seven peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having eight peaks selected from 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having a peak at 8.2±0.2, 9.0±0.2, 11.6±0.2, 14.4±0.2, 16.6±0.2, 20.7±0.2, 21.1±0.2, 22.2±0.2, and 24.5±0.2 °2θ (e.g., 8.2±0.1, 9.0±0.1, 11.6±0.1, 14.4±0.1, 16.6±0.1, 20.7±0.1, 21.1±0.1, 22.2±0.1, and 24.5±0.1 °2θ) using Cu Kα radiation.

In some embodiments, the compound (e.g., the crystalline form of the fumarate salt of Compound 7) is characterized by an XRPD pattern having a peak at from about 11.4 to about 11.8, from about 20.9 to about 21.3, and from about 24.3 to about 24.7 °2θ using Cu Kα radiation.

In some embodiments, the compound (e.g., the crystalline form of the fumarate salt of Compound 7) is characterized by an XRPD pattern having a peak at from about 11.4 to about 11.8, from about 16.4 to about 16.8, from about 20.9 to about 21.3, and from about 24.3 to about 24.7 °2θ using Cu Kα radiation.

In some embodiments, the compound (e.g., the crystalline form of the fumarate salt of Compound 7) is characterized by an XRPD pattern having a peak at from about 11.4 to about 11.8, from about 16.4 to about 16.8, from about 20.9 to about 21.3, from about about 22.0 to about 22.4, and from about 24.3 to about 24.7 °2θ using Cu Kα radiation.

In some embodiments, the compound (e.g., the crystalline form of the fumarate salt of Compound 7) is characterized by an XRPD pattern having a peak at from about 11.4 to about 11.8, from about 16.4 to about 16.8, from about 20.5 to about 20.9, from about 20.9 to about 21.3, from about about 22.0 to about 22.4, and from about 24.3 to about 24.7 °2θ using Cu Kα radiation.

In some embodiments, the compound (e.g., the crystalline form of the fumarate salt of Compound 7) is characterized by an XRPD pattern having a peak at from about 8.8 to about 9.2, from about 11.4 to about 11.8, from about 16.4 to about 16.8, from about 20.5 to about 20.9, from about 20.9 to about 21.3, from about about 22.0 to about 22.4, and from about 24.3 to about 24.7 °2θ using Cu Kα radiation.

In some embodiments, the compound (e.g., the crystalline form of the fumarate salt of Compound 7) is characterized by an XRPD pattern having a peak at from about 8.0 to about 8.4, from about 8.8 to about 9.2, from about 11.4 to about 11.8, from about 16.4 to about 16.8, from about 20.5 to about 20.9, from about 20.9 to about 21.3, from about about 22.0 to about 22.4, and from about 24.3 to about 24.7 °2θ using Cu Kα radiation.

In some embodiments, the compound (e.g., the crystalline form of the fumarate salt of Compound 7) is characterized by an XRPD pattern having a peak at from about 8.0 to about 8.4, from about 8.8 to about 9.2, from about 11.4 to about 11.8, from about 14.2 to about 14.6, from about 16.4 to about 16.8, from about 20.5 to about 20.9, from about 20.9 to about 21.3, from about about 22.0 to about 22.4, and from about 24.3 to about 24.7 °2θ using Cu Kα radiation.

In some embodiments, the compound (e.g., the crystalline form of the fumarate salt of Compound 7) is characterized by an XRPD pattern having a peak at from about 8.1 to about 8.3, from about 8.9 to about 9.1, from about 11.5 to about 11.7, from about 14.3 to about 14.5, from about 16.5 to about 16.7, from about 20.6 to about 20.8, from about 21.0 to about 21.2, from about about 22.1 to about 22.3, and from about 24.4 to about 24.6 °2θ using Cu Kα radiation.

In some embodiments, the compound (e.g., the crystalline form of the fumarate salt of Compound 7) is characterized by an XRPD pattern having a peak at about 8.2, about 9.0, about 11.6, about 14.4, about 16.6, about 20.7, about 21.1, about 22.2, and about 24.5 °2θ using Cu Kα radiation.

In some embodiments, the compound (e.g., the crystalline form of the fumarate salt of Compound 7) has an endothermic peak top temperature in differential scanning calorimeter (DSC) analysis at between about 210° C. and about 250° C., between about 215° C. and about 245° C., between about 220° C. and about 240° C., between about 225° C. and about 235° C., or between about 230° C. and about 232° C.

In some embodiments, the compound (e.g., the crystalline form of the fumarate salt of Compound 7) has an endothermic peak top temperature in differential scanning calorimeter (DSC) analysis at about 231.0° C.

### Compound 7 Fumarate Salt Type B

In some embodiments, the compound is a fumarate salt of Compound 7.

In some embodiments, the compound is a crystalline form of a fumarate salt of Compound 7.

In some embodiments, the compound (e.g., the crystalline form of the fumarate salt of Compound 7) is characterized by an XRPD pattern having at least one peak selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least two peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least three peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least four peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least five peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least six peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least seven peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least eight peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having at least nine peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having one peak selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having two peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having three peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having four peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having five peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having six peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having seven peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having eight peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having nine peaks selected from 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having a peak at 4.4±0.2, 7.5±0.2, 9.0±0.2, 11.7±0.2, 14.5±0.2, 16.7±0.2, 21.3±0.2, 22.2±0.2, 24.7±0.2, and 25.9±0.2 °2θ (e.g., 4.4±0.1, 7.5±0.1, 9.0±0.1, 11.7±0.1, 14.5±0.1, 16.7±0.1, 21.3±0.1, 22.2±0.1, 24.7±0.1, and 25.9±0.1 °2θ) using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, and from about 11.5 to about 11.9 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 11.5 to about 11.9, and from about 24.5 to about 24.9 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 11.5 to about 11.9, from about 21.1 to about 21.5, and from about 24.5 to about 24.9 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 11.5 to about 11.9, from about 16.5 to about 16.9, from about 21.1 to about 21.5, and from about 24.5 to about 24.9 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 8.8 to about 9.2, from about 11.5 to about 11.9, from about 16.5 to about 16.9, from about 21.1 to about 21.5, and from about 24.5 to about 24.9 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 8.8 to about 9.2, from about 11.5 to about 11.9, from about 16.5 to about 16.9, from about 21.1 to about 21.5, from about 24.5 to about 24.9, and from about 25.7 to about 26.1 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 8.8 to about 9.2, from about 11.5 to about 11.9, from about 16.5 to about 16.9, from about 21.1 to about 21.5, from about 22.0 to about 22.4, from about 24.5 to about 24.9, and from about 25.7 to about 26.1 °2θ using Cu Kα radiation.

In some embodiments, the compound (e.g., the crystalline form of the fumarate salt of Compound 7) is characterized by an XRPD pattern having a peak at from about 4.2 to about 4.6, from about 7.3 to about 7.7, from about 8.8 to about 9.2, from about 11.5 to about 11.9, from about 14.3 to about 14.7, from about 16.5 to about 16.9, from about 21.1 to about 21.5, from about 22.0 to about 22.4, from about 24.5 to about 24.9, and from about 25.7 to about 26.1 °2θ using Cu Kα radiation.

In some embodiments, the compound (e.g., the crystalline form of the fumarate salt of Compound 7) is characterized by an XRPD pattern having a peak at from about 4.3 to about 4.5, from about 7.4 to about 7.6, from about 8.9 to about 9.1, from about 11.6 to about 11.8, from about 14.4 to about 14.6, from about 16.6 to about 16.8, from about 21.2 to about 21.4, from about 22.1 to about 22.3, from about 24.6 to about 24.8, and from about 25.8 to about 26.0 °2θ using Cu Kα radiation.

In some embodiments, the compound (e.g., the crystalline form of the fumarate salt of Compound 7) is characterized by an XRPD pattern having a peak at about 4.4, about 7.5, about 9.0, about 11.7, about 14.5, about 16.7, about 21.3, about 22.2, about 24.7, and about 25.9 °2θ using Cu Kα radiation.

In some embodiments, the compound (e.g., the crystalline form of the fumarate salt of Compound 7) has an endothermic peak top temperature in differential scanning calorimeter (DSC) analysis at between about 45° C. and about 85° C., between about 50° C. and about 80° C., between about 55° C. and about 75° C., between about 60° C. and about 70° C., or between about 65° C. and about 67° C.

In some embodiments, the compound (e.g., the crystalline form of the fumarate salt of Compound 7) has an endothermic peak top temperature in differential scanning calorimeter (DSC) analysis at between about 105° C. and about 145° C., between about 110° C. and about 140° C., between about 115° C. and about 135° C., between about 120° C. and about 130° C., or between about 125° C. and about 127° C.

In some embodiments, the compound (e.g., the crystalline form of the fumarate salt of Compound 7) has an endothermic peak top temperature in differential scanning calorimeter (DSC) analysis at about 66.3° C. and/or at about 125.9° C.

### Compound 7 Fumarate Salt Type C

In some embodiments, the compound is a fumarate salt of Compound 7.

In some embodiments, the compound is a crystalline form of a fumarate salt of Compound 7.

In some embodiments, the compound (e.g., the crystalline form of the fumarate salt of Compound 7) is characterized by an XRPD pattern having at least one peak selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least two peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least three peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least four peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least five peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least six peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having at least seven peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having one peak selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having two peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having three peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having four peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having five peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having six peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having seven peaks selected from 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having a peak at 9.7±0.2, 12.2±0.2, 12.8±0.2, 13.6±0.2, 14.0±0.2, 22.5±0.2, 24.4±0.2, and 24.9±0.2 °2θ (e.g., 9.7±0.1, 12.2±0.1, 12.8±0.1, 13.6±0.1, 14.0±0.1, 22.5±0.1, 24.4±0.1, and 24.9±0.1 °2θ) using Cu Kα radiation.

In some embodiments, the compound (e.g., the crystalline form of the fumarate salt of Compound 7) is characterized by an XRPD pattern having a peak at from about 9.5 to about 9.9, from about 13.4 to about 13.8, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.5 to about 9.9, from about 12.0 to about 12.4, from about 13.4 to about 13.8, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.5 to about 9.9, from about 12.0 to about 12.4, from about 13.4 to about 13.8, from about 13.8 to about 14.2, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.5 to about 9.9, from about 12.0 to about 12.4, from about 12.6 to about 13.0, from about 13.4 to about 13.8, from about 13.8 to about 14.2, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.5 to about 9.9, from about 12.0 to about 12.4, from about 12.6 to about 13.0, from about 13.4 to about 13.8, from about 13.8 to about 14.2, from about 24.2 to about 24.6, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.5 to about 9.9, from about 12.0 to about 12.4, from about 12.6 to about 13.0, from about 13.4 to about 13.8, from about 13.8 to about 14.2, from about 22.3 to about 22.7, from about 24.2 to about 24.6, and from about 24.7 to about 25.1 °2θ using Cu Kα radiation.

In some embodiments, the compound (e.g., the crystalline form of the fumarate salt of Compound 7) is characterized by an XRPD pattern having a peak at from about 9.6 to about 9.8, from about 12.1 to about 12.3, from about 12.7 to about 12.9, from about 13.5 to about 13.7, from about 13.9 to about 14.1, from about 22.4 to about 22.6, from about 24.3 to about 24.5, and from about 24.8 to about 25.0 °2θ using Cu Kα radiation.

In some embodiments, the compound (e.g., the crystalline form of the fumarate salt of Compound 7) is characterized by an XRPD pattern having a peak at about 9.7, about 12.2, about 12.8, about 13.6, about 14.0, about 22.5, about 24.4, and about 24.9 °2θ using Cu Kα radiation.

In some embodiments, the compound (e.g., the crystalline form of the fumarate salt of Compound 7) has an endothermic peak top temperature in differential scanning calorimeter (DSC) analysis at between about 190° C. and about 230° C., between about 195° C. and about 225° C., between about 200° C. and about 220° C., between about 205° C. and about 215° C., or between about 210° C. and about 212° C.

In some embodiments, the compound (e.g., the crystalline form of the fumarate salt of Compound 7) has an endothermic peak top temperature in differential scanning calorimeter (DSC) analysis at about 211.5° C.

In some embodiments, one or more of the compounds of the present disclosure are selective inhibitors of EHMT1. In some embodiments, one or more of the compounds of the present disclosure are selective inhibitors of EHMT2. In some embodiments, one or more of the compounds of the present disclosure are inhibitors of EHMT1 and EHMT2.

In some aspects, the present disclosure provides pharmaceutical compositions comprising a compound of the present disclosure and a pharmaceutically acceptable carrier.

In some aspects, the present disclosure provides methods of inhibiting one or more HMTs (e.g., inhibiting one or both of EHMT1 and EHMT2), wherein the method comprises administering to a subject in need thereof a therapeutically effective amount of a compound of the present disclosure.

In some embodiments, the subject has an EHMT-mediated disorder (e.g., an EHMT1-mediated disorder, an EHMT2-mediated disorder, or an EHMT1/2-mediated disorder). In some embodiments, the subject has a blood disorder, for example, an anemia or thalassemia, e.g., sickle cell anemia. In some embodiments, the subject has a cancer.

In some aspects, the present disclosure provides methods of preventing or treating a blood disorder (e.g., via inhibition of a methyltransferase enzyme, e.g., of EHMT1 and/or EHMT2), the method comprising administering to a subject in need thereof a therapeutically effective amount of a compound provided herein. In some embodiments, the blood disorder is sickle cell anemia or 3-thalassemia. In some embodiments, the blood disorder is a cancer, e.g., a hematological cancer, such as, for example, a leukemia or a lymphoma.

In some aspects, the present disclosure provides methods of preventing or treating a cancer (e.g., via inhibition of a methyltransferase enzyme selected from EHMT1 and EHMT2), the method comprising administering to a subject in need thereof a therapeutically effective amount of a compound of the present disclosure. In some embodiments, the cancer is lymphoma, leukemia, melanoma, breast cancer, ovarian cancer, hepatocellular carcinoma, prostate carcinoma, lung cancer, brain cancer, or hematological cancer. In some embodiments, the cancer is melanoma. In some embodiments, the hematological cancer is acute myeloid leukemia (AML) or chronic lymphocytic leukemia (CLL). In some embodiments, the lymphoma is diffuse large B-cell lymphoma, follicular lymphoma, Burkitt's lymphoma or Non-Hodgkin's Lymphoma. In some embodiments, the cancer is chronic myelogenous leukemia (CML), acute myeloid leukemia (AML), acute lymphocytic leukemia (ALL), mixed lineage leukemia (MLL), or myelodysplastic syndromes (MDS).

In some aspects, the present disclosure provides one or more of the compounds described herein for use in inhibiting one or both of EHMT1 and EHMT2 in a subject in need thereof.

In some aspects, the present disclosure provides one or more of the compounds described herein for use in preventing or treating an EHMT-mediated disorder in a subject in need thereof.

In some aspects, the present disclosure provides one or more of the compounds described herein for use in preventing or treating a blood disorder in a subject in need thereof.

In some aspects, the present disclosure provides one or more of the compounds described herein for use in preventing or treating a cancer in a subject in need thereof.

In some aspects, the present disclosure provides uses of one or more of the compounds described herein in the manufacture of a medicament for inhibiting one or both of EHMT1 and EHMT2 in a subject in need thereof.

In some aspects, the present disclosure provides uses of one or more of the compounds described herein in the manufacture of a medicament for preventing or treating an EHMT-mediated disorder in a subject in need thereof.

In some aspects, the present disclosure provides uses of one or more of the compounds described herein in the manufacture of a medicament for preventing or treating a blood disorder in a subject in need thereof.

In some aspects, the present disclosure provides uses of one or more of the compounds described herein in the manufacture of a medicament for preventing or treating a cancer in a subject in need thereof.

In some aspects, the present disclosure provides of preparing one or more of the compounds described herein. In some embodiments, the methods comprise one or more of the steps in one or more of Schemes 1-10. In some embodiments, one or more of the compounds inhibit a kinase with an enzyme inhibition IC₅₀ value of about 100 nM or greater, 1 μM or greater, 10 μM or greater, 100 μM or greater, or 1000 μM or greater. In some embodiments, one or more of the compounds inhibit a kinase with an enzyme inhibition IC₅₀ value of about 1 mM or greater.

In some embodiments, one or more of the compounds inhibit a kinase with an enzyme inhibition IC₅₀ value of 1 μM or greater, 2 μM or greater, 5 μM or greater, or 10 μM or greater, wherein the kinase is one or more of the following: AbI, AurA, CHK1, MAP4K, IRAK4, JAK3, EphA2, FGFR3, KDR, Lck, MARK1, MNK2, PKCb2, SIK, and Src.

In some embodiments, compounds of the present disclosure that contain one or more nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structure of a compound represents a certain isomer for convenience in some cases, but the present disclosure includes all isomers, such as geometrical isomers, optical isomers based on an asymmetrical carbon, stereoisomers, tautomers, and the like.

In addition, a crystal polymorphism may be present for the compounds represented by the structure. It is noted that any crystal form, crystal form mixture, or anhydride or hydrate thereof is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed in this disclosure include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

A “Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. Examples of lactam-lactim tautomerism are as shown below.

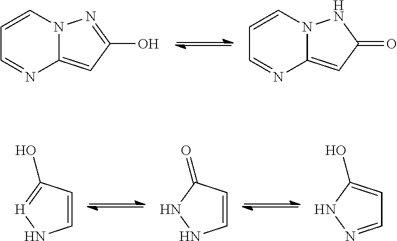

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The term “XRPD”, as used herein, refers to x-ray powder diffraction. In some embodiments, the x-ray powder diffraction is obtained using a Cu Kα radiation. In some embodiments, the x-ray powder diffraction has one or more peaks having determined 20 angles.

In some embodiments, the phrase “crystalline form of Compound A,” as used herein, refers to a crystalline form of a freebase of Compound A. In some embodiments, the freebase of Compound A is an anhydrate.

In some embodiments, the phrase “crystalline form of a Y salt of Compound X,” as used herein, refers to a crystalline form of a salt formed between Compound A and anion X. In some embodiments, the salt formed between Compound A and anion X is an anhydrate. In some embodiments, the ratio between Compound A and anion X in the salt is about 1:1, about 1:2, about 1:3, or about 1:4.

The compounds described herein include the compounds themselves, as well as their pharmaceutically acceptable salts, and their solvates, if applicable.

A “pharmaceutically acceptable salt”, for example, can be formed between an anion and a compound of the present disclosure. In some embodiments, the salt is formed between an anion and a positively charged group (e.g., amino) on a compound of the present disclosure. Suitable anions include adipate, glycoate, succinate, chloride, bromide, iodide, sulfate, bisulfate, sulfamate, nitrate, phosphate, oxalate, citrate, methanesulfonate, trifluoroacetate, glutamate, glucuronate, glutarate, malate, maleate, succinate, fumarate, tartrate, tosylate, salicylate, lactate, naphthalenesulfonate, hippurate, gentisate, benzoate, and acetate (e.g., trifluoroacetate). In some embodiments, the pharmaceutically acceptable salt is hydrochloride salt, sulfate salt, glycolate salt, adipate salt, succinate salt, oxalate salt, phosphate salt, fumarate salt, hippurate salt, gentisate salt, or benzoate salt. The term “pharmaceutically acceptable anion” refers to an anion suitable for forming a pharmaceutically acceptable salt.

Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14. For example, some embodiments of the present disclosure embrace compounds of the structures provided herein, wherein one or more of the hydrogens is substituted for deuterium or tritium.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

The term “about”, as used herein, refers to a range within 10%, 9%, 8%, 7%, 6%, 5%, 4%, 3%, 2%, 1%, 0.5%, 0.1%, 0.05%, or 0.01% devastations of the stated value. In some embodiments, and with regard to XRPD patterns, the term “about” refers to the stated value +/−0.5, +/−0.4, +/−0.3, +/−0.2, or +/−0.1 degrees 2θ. In some embodiments, and with regard to temperatures, the term “about” refers to the stated value +/−20° C., +/−15° C., +/−10° C., +/−8° C., +/−6° C., +/−5° C., +/−4° C., +/−3° C., +/−2° C., +/−1° C., or +/−0.5° C.

The present disclosure provides methods for the synthesis of the compounds described herein. The present disclosure also provides detailed methods for the synthesis of various disclosed compounds of the present disclosure according to any one of Schemes 1-9 shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable.

Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

Compounds of the present disclosure can be conveniently prepared by a variety of methods familiar to those skilled in the art. The compounds of the present disclosure disclosure may be prepared according to the procedures illustrated in Schemes 1-9 below, from commercially available starting materials or starting materials which can be prepared using literature procedures.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{d} edition, John Wiley & Sons: New York, 1999.

Preferred protecting groups include, but are not limited to:

For a hydroxyl moiety: TBS, benzyl, THP, Ac

For carboxylic acids: benzyl ester, methyl ester, ethyl ester, allyl ester

For amines: Cbz, BOC, DMB

For diols: Ac (×2) TBS (×2), or when taken together acetonides

For thiols: Ac

For benzimidazoles: SEM, benzyl, PMB, DMB

For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate theomers.

The following abbreviations are used throughout the specification and are defined below: ACN acetonitrile
Ac acetyl
AcOH acetic acid
AlCl3 aluminum chloride
BINAP (2,2′-bis(diphenylphosphino)-1,1′-binaphthyl)
t-BuOK potassium t-butoxide
tBuONa or t-BuONa sodium t-butoxide
br broad
BOC tert-butoxy carbonyl
Cbz benzyloxy carbonyl
CDCl3CHCl3 chloroform
CH2Cl2 dichloromethane
CH3CN acetonitrile
CsCO3 cesium carbonate
CH3NO3 nitromethane
d doublet
dd doublet of doublets
dq doublet of quartets
DCE 1,2 dichloroethane
DCM dichloromethane
Δ heat
δ chemical shift
DIEA N,N-diisopropylethylamine (Hunig's base)
DMB 2,4 dimethoxy benzyl
DMF N,N-Dimethylformamide
DMSO Dimethyl sulfoxide
DMSO-d6 deuterated dimethyl sulfoxide
EA or EtOAc Ethyl acetate
ES electrospray
Et3N triethylamine
equiv equivalents
g grams
h hours
H2O water
HCl hydrogen chloride or hydrochloric acid
HPLC High performance liquid chromatography
Hz Hertz
IPA isopropyl alcohol
i-PrOH isopropyl alcohol
J NMR coupling constant
K2CO3 potassium carbonate
HI potassium iodide
KCN potassium cyanide
LCMS or LC-MSLiquid chromatography mass spectrum
M molar
m multiplet
mg milligram
MHz megahertz
mL milliliter
mm millimeter
mmol millimole
mol mole
[M+1] molecular ion plus one mass unit
m/z mass/charge ratio
m-CPBA meta-chloroperbenzoic acid
MeCN Acetonitrile
MeOH methanol
MeI Methyl iodide
min minutes
μm micron
MsCl Mesyl chloride
MW microwave irradiation
N normal
Na2SO4 sodium sulfate
NH3 ammonia
NaBH(AcO)3 sodium triacetoxyborohydride
NaI sodium iodide
Na2SO4 sodium sulfate
NH4Cl ammonium chloride
NH4HCO3 ammonium bicarbonate
nm nanometer
NMP N-methylpyrrolidinone
NMR Nuclear Magnetic Resonance
Pd(OAc)2 palladium (II) acetate
Pd/C Palladium on carbon
Pd2(dba)3 Tris(dibenzylideneacetone)dipalladium(0)
PMB para methoxybenzyl
ppm parts per million
POCl3 phosphoryl chloride
prep-HPLC preparative High Performance Liquid Chromatography
PTSA para-toluenesulfonic acid
p-TsOH para-toluenesulfonic acid
RT retention time
rt room temperature
s singlet
t triplet
t-BuXPhos 2-Di-tert-butylphosphino-2′, 4′, 6′-triisopropylbiphenyl
TEA Triethylamine
TFA trifluoroacetic acid
TfO triflate
THP tetrahydropyran
TsOH tosic acid
UV ultraviolet

A person of ordinary skill in the art will recognize that in the above schemes the order of many of the steps are interchangeable.

Compounds of the present disclosure inhibit the histone methyltransferase activity of G9a, also known as KMT1C (lysine methyltransferase 1C) or EHMT2 (euchromatic histone methyltransferase 2), or a mutant thereof and, accordingly, in one aspect of the disclosure, certain compounds disclosed herein are candidates for treating, or preventing certain conditions, diseases, and disorders in which EHMT2 plays a role. The present disclosure provides methods for treating conditions and diseases the course of which can be influenced by modulating the methylation status of histones or other proteins, wherein said methylation status is mediated at least in part by the activity of EHMT2. Modulation of the methylation status of histones can in turn influence the level of expression of target genes activated by methylation, and/or target genes suppressed by methylation. The method includes administering to a subject in need of such treatment, a therapeutically effective amount of a compound of the present disclosure, or a pharmaceutically acceptable salt, polymorph, solvate, or stereoisomer thereof.

Unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

In still some aspects, this disclosure provides a method of modulating the activity of EHMT2, which catalyzes the dimethylation of lysine 9 on histone H3 (H3K9) in a subject in need thereof. In some embodiments, the method comprises contacting an EHMT2 protein with a compound provided herein in an amount effective to inhibit the H3K9 methyl transferase activity by at least 10%, at least 20%, at least 30%, at least 40%, at least 50%, at least 60%, at least 70%, at least 80%, at least 90%, at least 95%, at least 98%, or at least 99%. In some embodiments, the contacting is in vivo. In some embodiments, a method is provided that comprises administering to a subject in need thereof a therapeutically effective amount of a compound described herein, wherein the compound inhibits histone methyltransferase activity of EHMT2. In some embodiments, the subject has an EHMT2-mediated disease. In some embodiments, the subject has a cancer. In some embodiments, the subject has a blood disorder. In some embodiments, the blood disorder is an anemia. In some embodiments, the blood disorder is sickle cell anemia. In some embodiments, the blood disorder is a hematological cancer. In some embodiments, the subject expresses a mutant form of EHMT2.

In some embodiments, the subject has an EHMT2-mediated cancer. In some embodiments, of the cancer is leukemia, prostate carcinoma, hepatocellular carcinoma, or lung cancer.

In some embodiments, the compounds disclosed herein are useful for treating an EHMT2-mediated disease, e.g., an EHMT2-mediated cancer or blood disorder. In some embodiments, the cancer is a hematological cancer. In some embodiments, the blood disorder is an anemia, e.g., sickle cell anemia.

In some embodiments, the cancer is brain and central nervous system (CNS) cancer, head and neck cancer, kidney cancer, ovarian cancer, pancreatic cancer, leukemia, lung cancer, lymphoma, myeloma, sarcoma, breast cancer, and prostate cancer. In some embodiments, a subject in need thereof is one who had, is having or is predisposed to developing brain and CNS cancer, kidney cancer, ovarian cancer, pancreatic cancer, leukemia, lymphoma, myeloma, and/or sarcoma. Exemplary brain and central CNS cancer includes medulloblastoma, oligodendroglioma, atypical teratoid/rhabdoid tumor, choroid plexus carcinoma, choroid plexus papilloma, ependymoma, glioblastoma, meningioma, neuroglial tumor, oligoastrocytoma, oligodendroglioma, and pineoblastoma. Exemplary ovarian cancer includes ovarian clear cell adenocarcinoma, ovarian endomethrioid adenocarcinoma, and ovarian serous adenocarcinoma. Exemplary pancreatic cancer includes pancreatic ductal adenocarcinoma and pancreatic endocrine tumor. Exemplary sarcoma includes chondrosarcoma, clear cell sarcoma of soft tissue, ewing sarcoma, gastrointestinal stromal tumor, osteosarcoma, rhabdomyosarcoma, and not otherwise specified (NOS) sarcoma. In some embodiments, cancers to be treated by the compounds of the disclosure are non NHL cancers.

In some embodiments, the cancer is acute myeloid leukemia (AML) or chronic lymphocytic leukemia (CLL), medulloblastoma, oligodendroglioma, ovarian clear cell adenocarcinoma, ovarian endomethrioid adenocarcinoma, ovarian serous adenocarcinoma, pancreatic ductal adenocarcinoma, pancreatic endocrine tumor, malignant rhabdoid tumor, astrocytoma, atypical teratoid/rhabdoid tumor, choroid plexus carcinoma, choroid plexus papilloma, ependymoma, glioblastoma, meningioma, neuroglial tumor, oligoastrocytoma, oligodendroglioma, pineoblastoma, carcinosarcoma, chordoma, extragonadal germ cell tumor, extrarenal rhabdoid tumor, schwannoma, skin squamous cell carcinoma, chondrosarcoma, clear cell sarcoma of soft tissue, ewing sarcoma, gastrointestinal stromal tumor, osteosarcoma, rhabdomyosarcoma, or not otherwise specified (NOS) sarcoma. In some embodiments, the cancer is acute myeloid leukemia (AML), chronic lymphocytic leukemia (CLL), medulloblastoma, ovarian clear cell adenocarcinoma, ovarian endomethrioid adenocarcinoma, pancreatic ductal adenocarcinoma, malignant rhabdoid tumor, atypical teratoid/rhabdoid tumor, choroid plexus carcinoma, choroid plexus papilloma, glioblastoma, meningioma, pineoblastoma, carcinosarcoma, extrarenal rhabdoid tumor, schwannoma, skin squamous cell carcinoma, chondrosarcoma, ewing sarcoma, epithelioid sarcoma, renal medullary carcinoma, diffuse large B-cell lymphoma, follicular lymphoma or NOS sarcoma.

In some embodiments, the cancer is lymphoma, leukemia or melanoma. In some embodiments, the cancer is lymphoma, e.g., follicular lymphoma, diffuse large B-cell lymphoma (DLBCL), Burkitt's lymphoma, or Non-Hodgkin's Lymphoma. In some embodiments, the lymphoma is non-Hodgkin's lymphoma (NHL), follicular lymphoma or diffuse large B-cell lymphoma. In some embodiments, the leukemia is chronic myelogenous leukemia (CML), acute myeloid leukemia, acute lymphocytic leukemia or mixed lineage leukemia.

In some embodiments, the EHMT2-mediated disorder is a hematological disorder.

The compounds provided herein inhibit the histone methyltransferase activity of EHMT2 or a mutant thereof and, accordingly, the present disclosure also provides methods for treating conditions and diseases the course of which can be influenced by modulating the methylation status of histones or other proteins, wherein said methylation status is mediated at least in part by the activity of EHMT2. Modulation of the methylation status of histones can in turn influence the level of expression of target genes activated by methylation, and/or target genes suppressed by methylation. The method includes administering to a subject in need of such treatment, a therapeutically effective amount of a compound of the present disclosure.

As used herein, a “subject” is interchangeable with a “subject in need thereof”, both of which refer to a subject having a disorder in which EHMT2-mediated protein methylation plays a part, or a subject having an increased risk of developing such disorder relative to the population at large. In some embodiments, the subject is a human. In some embodiments, the subject is a non-human mammal. In some embodiments, the subject is a mammal. In some embodiments, the subject is a primate, mouse, rat, dog, cat, cow, horse, goat, camel, sheep or a pig. The subject can also be a bird or fowl. A subject in need thereof can be one who has been previously diagnosed or identified as having cancer or a precancerous condition. A subject in need thereof can also be one who has (e.g., is suffering from) cancer or a precancerous condition. In some embodiments, a subject in need thereof can be one who has an increased risk of developing such disorder relative to the population at large (i.e., a subject who is predisposed to developing such disorder relative to the population at large). A subject in need thereof can have a precancerous condition. A subject in need thereof can have refractory or resistant cancer (i.e., cancer that does not respond or has not yet responded to treatment). The subject may be resistant at start of treatment or may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In a preferred embodiment, the subject has cancer or a cancerous condition. In some embodiments, the cancer is leukemia, prostate carcinoma, hepatocellular carcinoma, and lung cancer.

As used herein, “candidate compound” refers to a compound of the present disclosure, or a pharmaceutically acceptable salt, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present disclosure, or a pharmaceutically acceptable salt, polymorph or solvate thereof. The biological or medical response can be the treatment of cancer. The biological or medical response can be treatment or prevention of a cell proliferative disorder. The biological response or effect can also include a change in cell proliferation or growth that occurs in vitro or in an animal model, as well as other biological changes that are observable in vitro. In vitro or in vivo biological assays can include, but are not limited to, enzymatic activity assays, electrophoretic mobility shift assays, reporter gene assays, in vitro cell viability assays, and the assays described herein.

For example, an in vitro biological assay that can be used includes the steps of (1) mixing a histone substrate (e.g., an isolated histone sample or an isolated histone peptide representative of human histone H3 residues 1-15) with recombinant EHMT2 enzymes; (2) adding a compound of the disclosure to this mixture; (3) adding non-radioactive and ³H-labeled S-Adenosyl methionine (SAM) to start the reaction; (4) adding excessive amount of non-radioactive SAM to stop the reaction; (4) washing off the free non-incorporated ³H-SAM; and (5) detecting the quantity of ³H-labeled histone substrate by any methods known in the art (e.g., by a PerkinElmer TopCount platereader).

For example, an in vitro study that can be used includes the steps of (1) treating cancer cells (e.g., breast cancer cells) with a compound of this disclosure; (2) incubating the cells for a set period of time; (3) fixing the cells; (4) treating the cells with primary antibodies that bind to dimethylated histone substrates; (5) treating the cells with a secondary antibody (e.g. an antibody conjugated to an infrared dye); (6) detecting the quantity of bound antibody by any methods known in the art (e.g., by a Licor Odyssey Infrared Scanner).

As used herein, “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

A compound of the present disclosure, or a pharmaceutically acceptable salt, polymorph or solvate thereof, can or may also be used to prevent a relevant disease, condition or disorder, or used to identify suitable candidates for such purposes. As used herein, “preventing,” “prevent,” or “protecting against” describes reducing or eliminating the onset of the symptoms or complications of such disease, condition or disorder.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press. Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

As used herein, “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

The present disclosure also provides pharmaceutical compositions comprising a compound described herein in combination with at least one pharmaceutically acceptable excipient or carrier.

A “pharmaceutical composition” is a formulation containing the compounds of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for administration of drugs, e.g., for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In some aspects, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1. e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, can be administered orally, nasally, transdermally, pulmonary, inhalationally, buccally, sublingually, intraperitoneally, subcutaneously, intramuscularly, intravenously, rectally, intrapleurally, intrathecally and/or parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated, the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Suitable techniques for formulation and administration of the disclosed compounds can be found in Remington: the Science and Practice of Pharmacy, 19^{th} edition, Mack Publishing Co., Easton, Pa. (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Example 1: Synthesis of Compound 1R ((R)-N2-(6-methoxy-5-((1-methylpyrrolidin-3-yl)methoxy)pyridin-3-yl)-N4,6-dimethylpyrimidine-2,4-diamine)

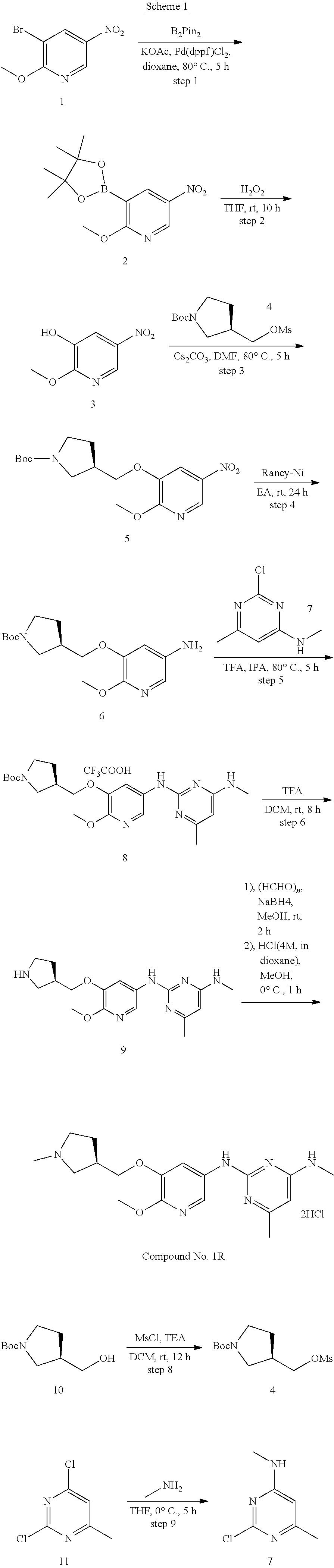

Steps 1 and 2.

Dioxane (10.4 L, 8 v), (1.3 kg, 1.0 eq.), KOAc (1.65 kg, 3.0 eq.), and B₂Pin₂ (1.7 kg, 1.2 eq.) were charged into 20 L reactor. Nitrogen was bubbled into the solution to remove any excess oxygen for 1 hour at 20˜30° C. Pd(dppf)Cl₂ (125.6 g, 0.03 eq.) was into the reactor into the mixture under nitrogen. The mixture was heated to 80˜90° C. The reaction mixture stirred for 3 hours at until HPLC showed the reaction was completed. The reaction mixture was cooled to 20˜30° C. and then filtered. The filtered cake was washed with dioxane (2.6 L, 2 v). The filtered solutions were combined and concentrated and then transferred to 20 L reactor. H₂O₂ (3.25 L, 2.5 v) was added at 20˜50° C., and the temperature was increased from 23 to 50° C. The reaction mixture was stirred for 30˜60 min until HPLC showed the reaction was completed. H₂O₂ (6.5 L, 5 v) was added in to the mixture, and the mixture was extracted with DCM (13.0 L, 10 v) twice. The organic phase was collected and washed with 15% brine (6.5 L, 5 v) twice, and was then extracted with 15% Na₂CO₃ (6.5 L, 5 L) twice. The aqueous phase was collected and the pH value was adjusted 10˜11 to 4˜5 with 3M HCl. The aqueous phase was then extracted with EA (13.0 L, 10.0 v) twice. The organic phase was collected and concentrated to about dryness, and heptane (6.5 L, 5.0 v) was added to slurry for 1 hour at 20˜30° C. The slurry was filtered, and the filtered cake was washed with heptane (650 ml, 0.5 v), drid in oven at 30˜40° C. for overnight to obtain 650.2 g product as brown solid with purity: 99.6%, yield: 67.8%.

Step 3.

DMF (9.0 L, 10.0 v), Cs₂CO₃ (3.5 kg, 2.0 eq.), 3 (900 g, 1.0 eq.), and 4 (1.5 kg, 1.0 eq.) were charged into the 20 L reactor. The mixture was heated to 80˜85° C. and was then stirred for 6 hours until HPLC showed the compound 3 less than 2.00% (1.6% was observed this time). The mixture was cooled to 20˜30° C. and then filtered. The filtered cake was washed with EA (18.0 L, 20.0 v). The filtered solutions were combined and washed with 15% brine (4.5 L, 5.0 v) for three times. The organic phase was concentrated under vacuum to dryness, yielding the product as brown solid (1808.0 g, purity: 97.8%, yield: 94.6%).

Step 4.

5 (900.0 g, 1.0 eq.), EtOAc (9.0 L, 10.0 v), and Pd/C (hydrous, 10% Pd loading, 45.0 g, 5% w/w) were charged into the 20 L pressure tank reactor. The reactor was evacuated and flushed three times with nitrogen. The reaction mixture was stirred for 16 hours by flushing with 5˜10 atmosphere of hydrogen at 20˜30° C. until sample for HPLC showed the reaction was completed. The reactor was evacuated and flushed three times with nitrogen. The mixture was filtered through diatomite, and the cake was washed with EtOAc (900 mL, 1.0 v). The filtered solutions were combined and concentrated to dryness under vacuum at 30˜40° C. to obtain the product as dark brown oil (1640.0 g, purity: 98.2%).

Step 5. 6 (794.4 g, 1.0 eq.) IPA (8.0 L, 5.0 v), and TFA (980.0 g, 2.0 eq.) were charged into the 50 L reactor. The reaction mixture was stirred for 30 min at room temperature. The solution of compound 7 (1630.0 g, 1.0 eq.) in IPA (13.0 L, 8.0 v) was charged into the reactor. The mixture was heated to 75˜85° C. and stirred for 1˜2 hours at 75˜85° C. until HPLC showed the reaction was completed. The mixture was cooled to 15˜25° C. and stirred for 2 hours at 15˜25° C. The mixture was then filtered, and the filtered cake was washed with heptane (1.6 L, 1.0 v) and dried in oven for 16 hours at 35˜45° C. to obtain the product as light brown solid (2006.0 g, purity: 95.7%).

Step 6.

8 (2000.0 g, 1.0 eq.), DCM (20.0 L, 10.0 v), and TFA (3509.0 g, 10.0 eq.) were charged into the 50 L reactor. The reaction mixture was stir for 16 hours at 20˜30° C. until HPLC showed the reaction was completed (no start material was observed). The reaction mixture was then concentrated to about dryness (light brown oil). MeOH (4.0 L, 2.0 v) was added to the mixture, and the mixture was stirred for 1˜2 hours. The mixture was filter, and the filtered cake was added in 50 L reactor with MeOH (10.0 L, 5.0 v) and H₂O (4.0 L, 2.0 v). The pH value of the mixture was adjusted to 11˜12 with 10% NaOH solution. The resulted mixture was extracted with DCM (16.0 L, 8.0 v) twice. The organic phases were combined, dried with Na2SO4 (2.0 kg), and filtered. The filtered solution was concentrated to dryness to yield the product as 1.1 kg light fink solid. (Purity: 98.8%, Yield: 90.2%).

Step 7.

9 (1.0 kg, 1.0 eq.), MeOH (10.0 L, 10.0 v), and (HCHO)ₙ (104.6 g, 1.2 eq.) were charged into the 20 L reactor. NaBH₄ (165.0 g, 1.5 eq.) was added to the mixture at the temperature below 30° C. A sample of the mixture was taken for HPLC, showing 3.0% of start material remained. NaHB₄ (33.0 g, 0.3 eq.) was further added to the mixture at the temperature below 30° C. A sample of the mixture was taken for HPLC, again showing 3.0% of start material remained. The reaction was quenched with aqueous of NH4Cl (8.0 L, 8.0 v) for more than 2 hours. The pH value of the mixture was Adjusted to 9˜10 with 10% NaOH aqueous solution, and the mixture was then stirred for 1 hour. The mixture was extracted with EA (10.0 L, 10.0 v) twice. The poranic phases were combined and concentrated under vacuum to dryness. The crude product was purified by chromatography with EA: MeOH: TEA (50:1:0.005˜10:1:0.005) to yield 750.0 g of freebase of Compound 1R as yellow solid (Purity: 98.9%).

Freebase Type A.

Compound 1R Freebase Type A was found to be poorly crystalline by XRPD. TGA curve showed 7.9% weight loss by 100° C. DSC curve displayed a broad endotherm around 86.9° C., and a possible exotherm at 154.5° C. followed by another endotherm at 199.1° C. (peak). The sample is likely a solvate/hydrate. Observed reversible heat flow in a DSC curve displayed a possible melting endotherm around 208.0° C. (peak). Birefringent rod-like crystals and amorphous material were observed for Compound 1R Freebase Type A under PLM.

Freebase Type B.

Compound 1R Freebase Type B was obtained by slurry of Compound 1R Freebase Type A in EtOAc. The sample was found to be crystalline by XRPD. Major XRPD diffraction peaks are showed in Table 2-1. TGA showed a weight loss of 4.4% up to 150° C. DSC curve showed multiple endotherms and exotherms. Scale up of Compound 1R Freebase Type B to 100 mg was successfully achieved by slurry of Type A in acetone. The Compound 1R Freebase Type B scale up batch showed similar XRPD pattern with the initial hit. Birefringent irregular shaped crystals were observed for Compound 1R Freebase Type B scale up sample under PLM.

TABLE 2-1
Major XRPD diffraction peaks of
Compound 1R Freebase Type B

| Peak No. | 2θ Position [°] | Intensity [cts] |
|---|---|---|
| 1 | 6.393 | 981.7 |
| 2 | 11.796 | 243.4 |
| 3 | 14.1961 | 317.5 |
| 4 | 18.207 | 316.9 |
| 5 | 19.146 | 277.9 |
| 6 | 25.668 | 1509.5 |
| 7 | 26.412 | 791.6 |
| 8 | 29.308 | 209.0 |

Step 7 Continued.

The pure freebase of Compound 1R (700.0 g, 1.0 eq.) and MeOH (5.6 L, 8.0 v) were charged into the 10 L reactor. The mixture was stirred for 15˜30 min until the mixture was dissolved. The formed solution was filtered, and the filtered solid was washed with MeOH (1.4 L, 2.0 v). The filtered solutions were combined, transferred to 20 L reactor, and cooled to 0˜10° C. A mixture of HCl and EA (2.0 M/L, 2.44 L) was added to dropwise for about 1 hour at 0˜10° C. The resulting mixture was then diluted with MeOH (3.5 L, 5.0 v) at 0˜10° C., stirred for 1 hour at 0˜10° C., and filtered. The filtered cake was slurried with EA (5.6 L, 8.0 v) for 1 hour at room temperature and was then filtered. The filtered cake was washed with EA (1.4 L, 2.0 v) and dried under vacuum at 60° C. for 24 hours to yield 640.0 g of hydrochloride salt of Compound 1R as off-white solid (Purity: 99.1%).

Hydrochloride Salt Type A.

Compound 1R Hydrochloride Salt Type A was found to be crystalline by XRPD. Major XRPD diffraction peaks are showed in Table 2-2. TGA curve showed 3.9% weight loss up to 100° C. DSC curve displayed a broad endotherm around 72.7° C. and a possible melting endotherm at 249.6° C. (peak) accompanied with decomposition. The sample is likely a solvate/hydrate. Birefringent and irregular shaped crystals were observed for Compound 1R Hydrochloride Salt Type A under PLM. DVS of Hydrochloride Salt Type A showed ˜8% water uptake at 25° C./80%, RH, indicating that Hydrochloride Salt Type A is hygroscopic. No change in XRPD pattern was observed for Hydrochloride Salt Type A before and after DVS.

TABLE 2-2
Major XRPD diffraction peaks of Compound 1R
Hydrochloride Salt Type A

| Peak No. | 2θ Position [°] | Intensity [cts] |
|---|---|---|
| 1 | 6.193 | 228.5 |
| 2 | 7.215 | 224.7 |
| 3 | 7.997 | 436.4 |
| 4 | 8.830 | 345.9 |
| 5 | 12.423 | 342.8 |
| 6 | 13.259 | 164.9 |
| 7 | 17.654 | 139.4 |
| 8 | 26.204 | 614.9 |

### Preparation of Intermediates

Step 8. DCM (10.4 L, 8 v), 10 (1300 g, 1.0 eq.), and TEA (848.3 g, 1.3 eq.) were charged into 20 L reactor. The mixture was colled to 0˜5° C. A solution of MsCl (812.4 g, 1.1 eq.) in DCM (2.6 L, 2 v) was added to the mixture dropwise. The temperature was observed to increase from 3° C. to 7° C. The mixture was stirred for 1 hour at 5˜25° C. until the reaction was completed (showed by LCMS). The reaction was quenched by water (65 ml, 0.5 eq.), and the resulting mixture was concentrated to about dryness. EA (13.0 L, 10 v) was added into the mixture. The mixture was then filtered, and the filtered cake was washed with EA (1.3 L, 1 v). The organic phases were combined, washed with 15% w/w brine (6.5 L, 5 v) for three times, and then concentrated to about 1 v. n-Heptane (13 L, 10 v) was added into the mixture, and the mixture was stirred for 2 hours at 20˜30° C. The mixture was filtered. The filtered cake was washed with n-heptane (0.65 L, 0.5 v) and then dried at 25˜35° C. for 16 hours to yield 1550 g of product as white solid with purity (98.1%), yield (84.2%).

Step 9.

Acetonitrile (12.0 L, 12 v) and 2,4-dichloro-6-methylpyrimidine (1.0 kg, 1.0 eq.) were charged into the 20 L reactor. The mixture was colled to 0˜5° C., and K2CO3 (2.5 kg, 3.0 eq.) and CH3NH2.HCl (497.0 g, 1.2 eq.) were charged into the mixture. The mixture was stirred overnight (about 16 h) at room temperature. A sample was taken for LCMS Analysis, showing the remaining start material was less than 0.5%. The reaction mixture was filtered, and the filtered cake was washed with EA (500 mL, 0.5 v). The filtered solutions were combined and concentrated to about 2˜3 v, then diluted with EA (10.0 L, 10 v). The resulted solution was washed with half brine (5 v) twice. The organic phase was collected and concentrated to about dryness (combined with three other batches). The resulted cake was charged into TBME (46.4 L, 8.0 v), and the mixture was slurried at 45˜50° C. for about 8 hours until theomer was less than 1.0%. The mixture was cooled to about 30° C. and then filtered. The filtered cake was washed with TBME (5.8 L, 0.1 v) and then dried at 30˜40° C. for 16 hours to yield 2.4 kg of product as off-white solid (purity: 99.8%, yield: 42.9%).

### Example 2: Synthesis of Compound 1S ((S)-N2-(6-methoxy-5-((l-methylpyrrolidin-3-yl)methoxy)pyridin-3-yl)-N4,6-dimethylpyrimidine-2,4-diamine)

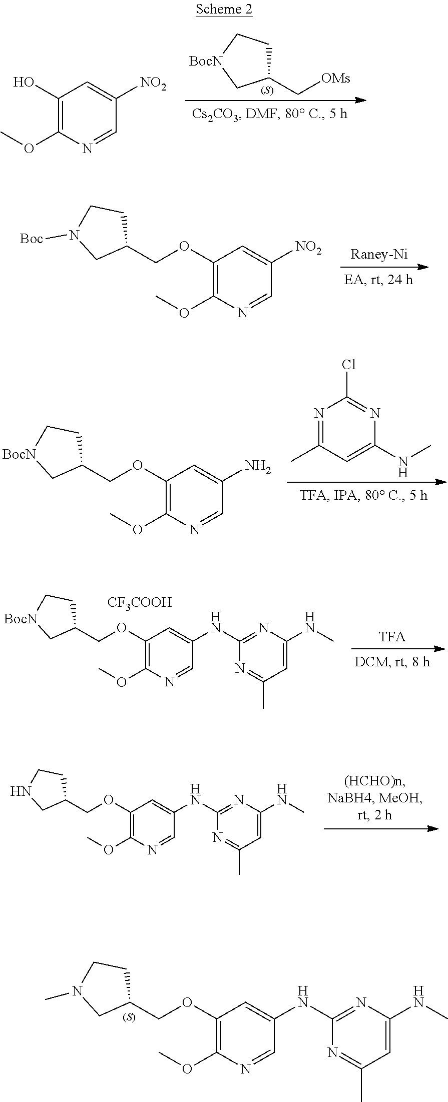

Compound 1R was synthesized according to the Scheme 2 illustrated above.

### Example 3: Synthesis of Compound 2 (6-methoxy-N-methyl-7-(3-(pyrrolidin-1-yl)propoxy)-4-(tetrahydro-2H-pyran-4-yl)quinolin-2-amine)

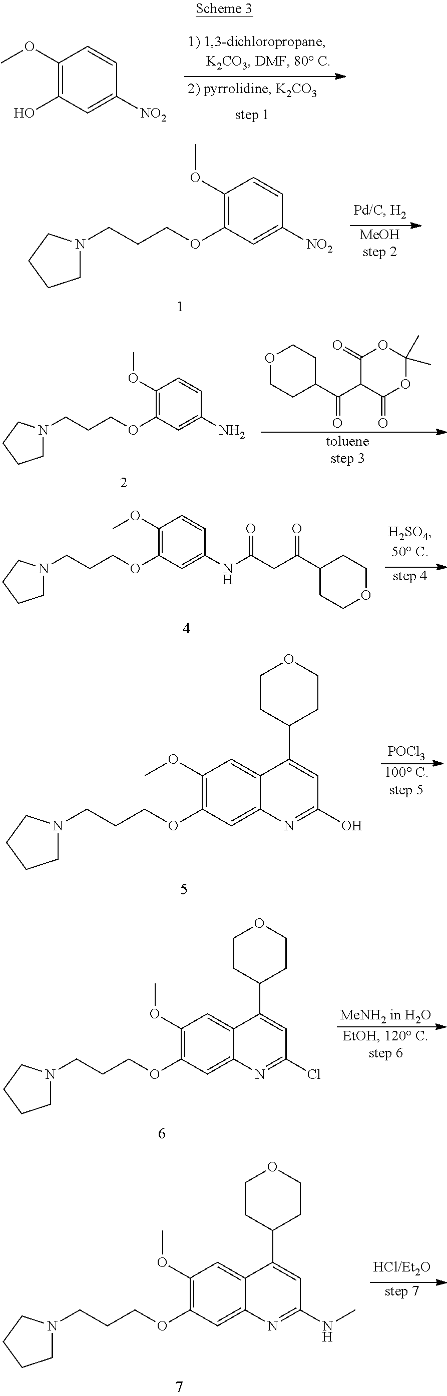

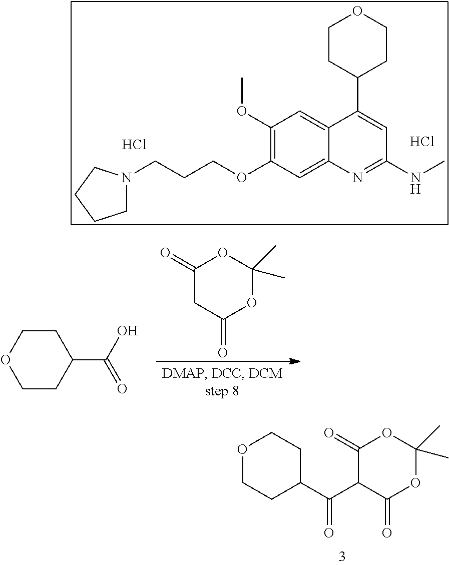

Step 1.

Into a 20-L 4-necked round-bottom flask, was placed 2-methoxy-5-nitrophenol (1090 g, 6.44 mol, 1.00 equiv), 1,3-dichloropropane (867 g, 1.20 equiv), potassium carbonate (1780 g, 12.88 mol, 2.00 equiv), N,N-dimethylformamide (10 L). The mixture was stirred at 80° C. When TLC indicated the material was totally consumed, recovered to room temperature. This was followed by the addition of potassium carbonate (1780 g, 12.88 mol, 2.00 equiv), pyrrolidine (915 g, 2.00 equiv). The resulting solution was stirred for 2 h at 80 degrees C. The resulting solution was diluted with 10 L of water. The resulting solution was extracted with 3×10 L of ethyl acetate and the organic layers combined. The resulting mixture was washed with 3×3 L of saturated sodium chloride aqueous. The mixture was dried over anhydrous sodium sulfate. The residue was applied onto a silica gel column with ethyl acetate (100%). The resulting solids were stirred in PE for overnight. The solids were collected by filtration. This resulted in 850 g (47%) of 1-[3-(2-methoxy-5-nitrophenoxy)propyl]pyrrolidine as a yellow solid. LC-MS: (ES, m/z): 281 [M+1].

Step 2.

Into a 3-L 3-necked round-bottom flask purged and maintained with an inert atmosphere of hydrogen, was placed 1-[3-(2-methoxy-5-nitrophenoxy)propyl]pyrrolidine (250 g, 891.84 mmol, 1.00 equiv), methanol (1.5 L), Palladium carbon (50 g). The resulting solution was stirred for 2 h at room temperature. Take three batches in parallel. The solids were filtered out. The resulting mixture was concentrated under vacuum. This resulted in 640 g of 4-methoxy-3-[3-(pyrrolidin-1-yl)propoxy]aniline as a red oil. LC-MS: (ES, m/z): 251 [M+1].

Step 3.

Into a 5000-mL round-bottom flask, was placed 4-methoxy-3-[3-(pyrrolidin-1-yl)propoxy]aniline (640 g, 2.56 mol, 1.00 equiv), 2,2-dimethyl-5-[(oxan-4-yl)carbonyl]-1,3-dioxane-4,6-dione (786 g, 3.07 mol, 1.20 equiv), toluene (5 L). The resulting solution was stirred for 2 h at 100° C. The resulting solution was diluted with 3 L of hydrogen chloride (2M) and the aqueous layers combined. The pH value of the solution was adjusted to 8 with sodium bicarbonate. The resulting solution was extracted with 3×3 L of dichloromethane and the organic layers combined and dried over anhydrous sodium sulfate and concentrated under vacuum. This resulted in 470 g (45%) of N-[4-methoxy-3-[3-(pyrrolidin-1-yl)propoxy]phenyl]-3-(oxan-4-yl)-3-oxopropanamide as a yellow solid. LC-MS: (ES, m/z): 405 [M+1].

Step 4.

Into a 5-L plastic beaker, was placed N-[4-methoxy-3-[3-(pyrrolidin-1-yl)propoxy]phenyl]-3-(oxan-4-yl)-3-oxopropanamide (470 g, 1.16 mol, 1.00 equiv), Con.H₂SO₄ (2 L). The resulting solution was stirred for 1 h at 50° C. in a water bath. The resulting solution was pour into ice. The pH value of the solution was adjusted to 9 with sodium hydroxide. The resulting solution was extracted with 3×3 L of dichloromethane and the organic layers combined and dried over anhydrous sodium sulfate and concentrated under vacuum. This resulted in 320 g (71%) of 6-methoxy-4-(oxan-4-yl)-7-[3-(pyrrolidin-1-yl)propoxy]quinolin-2-ol as a yellow solid. LC-MS: (ES, m/z): 387 [M+1].

Step 5.

Into a 3-L 4-necked round-bottom flask, was placed 6-methoxy-4-(oxan-4-yl)-7-[3-(pyrrolidin-1-yl)propoxy]quinolin-2-ol (320 g, 827.98 mmol, 1.00 equiv), POCl3 (1 L). The resulting solution was stirred for 2 h at 100° C. in an oil bath. The resulting mixture was concentrated under vacuum. The rest phosphorus oxychloride was pour into ice. The pH value of the solution was adjusted to 8 with sodium bicarbonate. The resulting solution was extracted with 3×2 L of dichloromethane and the organic layers combined and dried over anhydrous sodium sulfate. This resulted in 250 g (75%) of 2-chloro-6-methoxy-4-(oxan-4-yl)-7-[3-(pyrrolidin-1-yl)propoxy]quinoline as a yellow solid. LC-MS: (ES, m/z): 405 [M+1].

Step 6.

Into a 2-L pressure tank reactor, was placed 2-chloro-6-methoxy-4-(oxan-4-yl)-7-[3-(pyrrolidin-1-yl)propoxy]quinoline (250 g, 617.39 mmol, 1.00 equiv), ethanol (500 mL), NH2Me in water (300 mL). The resulting solution was stirred for 3 days at 120° C. The resulting mixture was concentrated under vacuum. The resulting solution was diluted with 2 L of water. The resulting solution was extracted with 3×2 L of dichloromethane and the organic layers combined and dried over anhydrous sodium sulfate and concentrated under vacuum. The resulting solids was stirred in methanol for 1 h. The solids were collected by filtration. This resulted in 130 g (53%) of freebase of Compound 2 as a gray solid. LC-MS: (ES, m/z): 400 [M+1].

### Freebase Type A.

Compound 2 was found to be crystalline by XRPD and assigned as Compound 2 Freebase Type A. Major XRPD diffraction peaks are showed Table in 3-1. TGA results show a weight loss of 0.2% up to 100° C. and 1.2% up to 200° C., and DSC curve displayed a melting endotherm at 179.2° C. (onset temperature). Birefringent, rod-like crystals were observed for Type A under PLM. DVS of Type A sample showed around 1.0% water uptake from 0 to 80% RH as evidenced by Error! Reference source not found, indicating that Type A is slightly hygroscopic. No form change was observed after DVS test as shown in XRPD overlay.

TABLE 3-1
Major XRPD diffraction peaks of
Compound 2 Freebase Type A

| Peak No. | 2θ Position [°] | Intensity [cts] |
|---|---|---|
| 1 | 7.977 | 2262.3 |
| 2 | 9.559 | 1272.8 |
| 3 | 12.594 | 1943.2 |
| 4 | 15.680 | 1720.1 |
| 5 | 15.972 | 768.5 |
| 6 | 18.622 | 700.8 |
| 7 | 19.182 | 1168.3 |
| 8 | 19.570 | 7150.8 |
| 9 | 23.190 | 782.8 |
| 10 | 30.041 | 1094.5 |

Step 7.

Into a 3-L round-bottom flask, was placed 6-methoxy-N-methyl-4-(oxan-4-yl)-7-[3-(pyrrolidin-1-yl)propoxy]quinolin-2-amine (130 g, 325.39 mmol, 1.00 equiv), methanol (200 mL), hydrogen chloride/Et2O (500 mL). The resulting solution was stirred for 20 min at room temperature. The solids were collected by filtration. The solid was dried in an oven under reduced pressure. This resulted in 122 g (79%) of dihydrochloride salt of Compound 2 as an off-white solid. LC-MS: (ES, m/z): 400 [M+1]. ¹H NMR (400 MHz, Deuterium Oxide) δ 7.16 (s, 1H), 7.10 (s, 1H), 6.62 (s, 1H), 4.20 (t, J=5.7 Hz, 2H), 4.03 (dd, J=11.7, 3.8 Hz, 2H), 3.87 (s, 3H), 3.71-3.60 (m, 4H), 3.42-3.27 (m, 3H), 3.11-2.96 (m, 5H), 2.25 (p, J=6.1 Hz, 2H), 2.16-2.02 (m, 2H), 1.97 (ddd, J=13.3, 8.6, 4.6 Hz, 2H), 1.78 (d, J=13.4 Hz, 2H), 1.76-1.68 (m, 1H), 1.66 (dd, J=12.8, 4.1 Hz, 1H).

Hydrochloride Salt Type A.

Compound 2 Hydrochloride Salt Type A was found to be crystalline by XRPD. Major XRPD diffraction peaks were showed in Table 3-2. TGA results show a weight loss of 3.8% up to 100° C., and shows an endotherm at 139.9° C. (onset) accompanied with possible decomposition. Birefringent irregular shaped crystals were observed Hydrochloride Salt Type A under PLM.

TABLE 3-2
Major XRPD diffraction peaks of Compound 2
Hydrochloride Salt Type A

| Peak No. | 2θ Position [°] | Intensity [cts] |
|---|---|---|
| 1 | 5.290 | 871.4 |
| 2 | 8.324 | 378.9 |
| 3 | 9.874 | 531.9 |
| 4 | 16.673 | 287.1 |
| 5 | 17.507 | 819.1 |
| 6 | 20.298 | 464.2 |
| 7 | 25.100 | 256.9 |
| 8 | 27.074 | 265.7 |

### Synthesis of Intermediate

Step 8. Into a 10-L 4-necked round-bottom flask, was placed oxane-4-carboxylic acid (500 g, 3.84 mol, 1.00 equiv), dichloromethane (4 L), 2,2-dimethyl-1,3-dioxane-4,6-dione (609 g, 4.23 mol, 1.10 equiv), 4-dimethylaminopyridine (704 g, 5.76 mol, 1.50 equiv). This was followed by the addition of a solution of DCC (800 g, 3.88 mol, 1.01 equiv) in dichloromethane (1000 mL) dropwise with stirring at 0 degrees C. The resulting solution was stirred for 14 h at room temperature. The solids were filtered out. The resulting solution was washed with 3×2 L 2M hydrochloride acid. Then washed with 3×2 L of saturated sodium chloride aqueous. The resulting solution was dried over anhydrous sodium sulfate and concentrated under vacuum. This resulted in 900 g (91%) of 2,2-dimethyl-5-[(oxan-4-yl)carbonyl]-1,3-dioxane-4,6-dione as a yellow solid.

LC-MS: (ES, m/z): 255 [M−1].

### Example 4: Synthesis of Compound 3 (N2-(4-methoxy-3-(((2-(pyrrolidin-1-yl)ethyl)amino)-methyl)phenyl)-N4,6-dimethylpyrimidine-2,4-diamine)

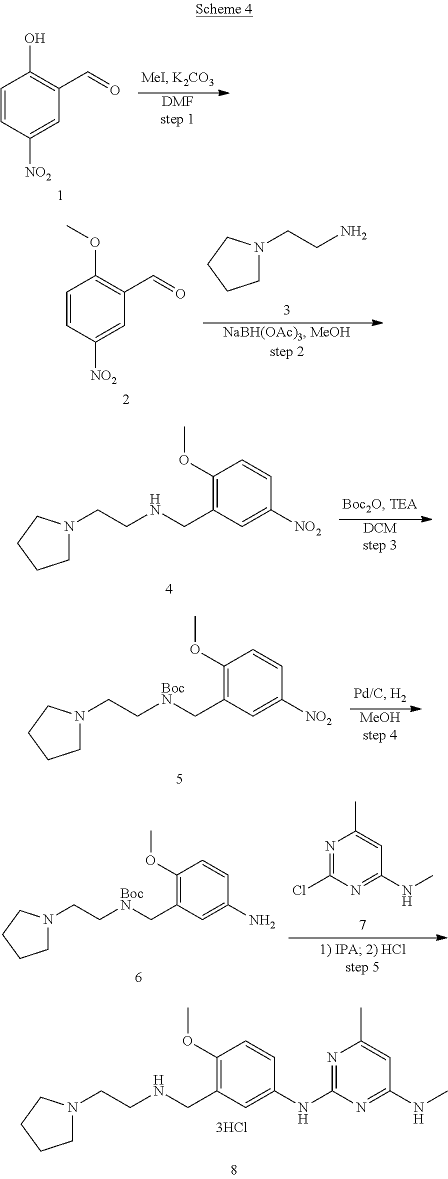

Step 1:

Into a 20 L round-bottom flask was charged 2-hydroxy-5-nitrobenzaldehyde (1.6 kg, 1.0 eq) and DMF (8.0 L, 5.0 v/w). K₂CO₃ (2.6 kg, 2.0 eq.) was added into the mixture followed by the dropwise addition of CH₃I (1.5 kg, 1.1 eq.) at 15˜35° C. The reaction was heated to 40-50° C. The reaction was monitored by HPLC until 2-hydroxy-5-nitrobenzaldehyde was present in less than 5.0%. Water (16 L, 10 v/w) was added into the mixture. The reaction was stirred for 12 hours at 20±5° C. The reaction was filtered and the filter cake was washed with water (3.2 L, 2.0 v/w) twice. The filter cake was collected and dried under vacuum at 40-50° C. This resulted in an off-white product (1.52 kg, purity: 99.7%, yield: 87.4%).

Step 2:

The product of step 1 and MeOH were charged into a 50 L reaction kettle. 2-(pyrrolidin-1-yl)ethan-1-amine and 3, (960 g, 1.0 eq.) were added to the reaction mass at 20-25° C. The reaction mixture was stirred for one hour and slowly charged with NaB(OAc)₃ (5.3 kg, 3.0 eq.) at 20-25° C. The reaction was stirred for two hours at 20-25° C. The reaction was monitored by HPLC until 2 was present in less than 3%. 10% NaOH (aq.) was charged into the reaction at 15-20° C. and the mixture was stirred for 30 minutes. The pH value of the solution was adjusted to 8-9. The reaction mixture was concentrated under vacuum at 35-40° C. to about 10 v. The resulting solution was extracted once with DCM (9.0 L, 6.0 v/w) and the organic layer was collected. HCl (3 N)(aq) was added to the organic layer at 15-20° C. and stirred for 30 minutes. The reaction reached a pH of 5-6. The mixture was separated and the aqueous layer was collected. The aqueous layer was washed once with DCM (6.0 L, 4.0 v/w). To the aqueous layer was added DCM (7.5 L, 5.0 v/w) and the pH of the aqueous layer was adjusted to a pH of 8-9 with sodium carbonate. The mixture was separated and the organic layer was collection. The aqueous layer was extracted with DCM (4.5 L, 3.0 v/w) once and the organic layer was collected. The organic layers were combined and concentrated under vacuum at 35-40° C. to about 3-4 v. The resultant solution was charged with PE (7.5 L, 5.0 v/w) and concentrated to about 3-4 v. The solution was stirred for two hours at 25±5° C. The reaction was filtered and the filter cake was washed with PE (2.3 L, 1.5 v/w) twice. The filter cake was dried under vacuum at 40-50° C. to obtain a yellow, solid product (1.6 kg, purity: 98.8%, yield: 67.4%).

Step 3.

4 (1.6 kg, 1.0 eq.) and DCM (16.0 L, 10 v/w) were charged into a 50 L reaction reactor. TEA (1.2 kg, 2.0 eq.) was added to the reaction mass at 20-25° C. Boc₂O (1.4 kg, 1.0 eq.) was added dropwise into the mixture at 15-25° C. The reaction was stirred for 16 hours at 20-25° C. The reaction was monitored by HPLC until 4 was present in less than 3%. The reaction mixture was washed with water (10.0 L, 6.0 v/w) and the organic layer was collected. The organic layer was washed with 20% NaCl aq. (6.5 L, 4.0 v/w) and the organic layer was collected. The organic layer was concentrated under vacuum at 35-40° C. to about 3-4 v. n-heptane was added (8.0 L, 5.0 v/w) to the solution and concentrated to about 3-4 v. The solution was stirred for 3 hours at 25±+5° C. The solution was filtered and the filter cake was washed with 3.2 L n-heptane. The filter cake was dried under vacuum at 40-50° C. to obtain the product as a yellow solid (2.0 kg, purity: 98.6%, yield: 88.0%).

Step 4:

To a 20 L reaction autoclave was charged 5 (800 g, 1.0 eq.) and MeOH (8.0 L, 10 v/w). Pd/C (40.0 g, 5.0%) was added to the reaction mass at 20-25° C. under N2 at a constant H2 pressure of 10-15 atm. The reaction was stirred for 16 hours at 20-25° C. The reaction was monitored by HPLC until 5 was present in less than 2%. The reaction mixture was filtered and the filter cake was washed twice with MeOH (0.8 L, 1.0 v/w). The filter solution was concentrated under vacuum at 35-40° C. to dryness. The product was obtained as a yellow oil (725.0 g, purity: 98.5%, yield: 96.9%).

Step 5.

To a 20 L reaction reactor was charged 6 (670.0 g, 1.0 eq.) and IPA (670 ml, 10 v/w). 7 (302.0 g, 1.0 eq.) was added to the reaction mass at 20-25° C. HCl in IPA (4M) (956 ml, 2.0 eq.) was added to reaction mass at 20-25° C. The reaction mixture was heated to 80-85° C. and stirred for 12 hours at the same temperature. The reaction was monitored by HPLC until 6 was present in less than 3%. The reaction was cooled to 30-40° C. and charged with HCl in IPA (4M/L) (717 ml, 1.5 eq.) at the same temperature. The reaction was stirred for 4 hours at 30-40° C. The reaction was filtered and the filter cake was washed twice with EtOAc (1.0 L, 1.5 v/w). The filter cake was dried under vacuum at 50-60° C. to obtain an off-white solid product (741 g, purity: 100%, yield: 80.3%).

Freebase Type A.

Compound 3 Freebase Type A from the typical synthetic procedure was found to be crystalline by XRPD. Major XRPD diffraction peaks are showed in Table 4-1Error! Reference source not found. TGA result showed a weight loss of 4.4% up to 130° C. DSC curve showed a broad endotherm at 93.2° C. (peak) possibly related to weight loss and a possible melting endotherm at 151.6° C. (peak) followed by decomposition. Birefringent irregular shape crystals were observed for Compound 3 Freebase Type A under PLM. DVS of Compound 3 Freebase Type A sample showed around 12.9% water uptake from 0% to 80% RH, indicating that Compound 3 Freebase Type A is hygroscopic. No form change was observed after DVS test as shown in XRPD.

TABLE 4-1
Major XRPD diffraction peaks of
Compound 3 Freebase Type A

| Peak No. | 2θ Position [°] | Intensity [cts] |
|---|---|---|
| 1 | 6.269 | 502.5 |
| 2 | 8.343 | 356.2 |
| 3 | 12 408 | 244.9 |
| 4 | 14.734 | 464.3 |
| 5 | 15.943 | 338.5 |
| 6 | 17.276 | 238.8 |
| 7 | 23.067 | 241.2 |
| 8 | 25.637 | 1045.3 |
| 9 | 32.739 | 586.9 |

Hydrochloride Salt Type A.

Compound 3 Hydrochloride Salt from synthesis batch was found to be crystalline by XRPD and assigned as Compound 3 Hydrochloride Salt Type A. Major XRPD diffraction peaks are showed in Table 4-2. TGA result shows a weight loss of 8.8% up to 100° C. DSC curve shows a broad desolvation/dehydration endotherm at 95.5° C. followed by likely melting-crystallizaiton-melting transitions, indicating that Compound 3 Hydrochloride Salt Type A is likely a solvate/hydrate.

TABLE 4-2
Major XRPD diffraction peaks of Compound 3
Hydrochloride Salt Type A

| Peak No. | 2θ Position [°] | Intensity [cts] |
|---|---|---|
| 1 | 6.816 | 1111.1 |
| 2 | 9.003 | 686.5 |
| 3 | 11.804 | 276.8 |
| 4 | 16.297 | 288.4 |
| 5 | 25.0519 | 354.3 |
| 6 | 25.559 | 833.2 |
| 7 | 26.330 | 280.5 |
| 8 | 27.613 | 524.9 |

Hydrochloride Salt Types B and C.

A sample of Compound 3 Hydrochloride Salt Type A was heated to 210° C. and cooled to 25° C. and the resulting solid showed a different XRPD pattern, which is mostly likely an anhydrous was assigned as Compound 3 Hydrochloride Salt Type B. Major XRPD diffraction peaks were showed in Table 4-3. DSC curve displayed one possible melting endotherm at 256.0° C. (peak). Compound 3 Hydrochloride Salt Type B is considered to be an anhydrate. DVS of Compound 3 Hydrochloride Salt Type B sample shows a water uptake of 15.3% at 25° C./80% RH, indicating that Compound 3 Hydrochloride Salt Type B is hygroscopic. A change of XRPD pattern was observed for Compound 3 Hydrochloride Salt Type B before and after DVS. Compound 3 Hydrochloride Salt after DVS showed the same pattern with the sample of Compound 3 Hydrochloride Salt Type A after DVS, suggesting the existence a new hydrate form, classified as Compound 3 Hydrochloride Salt Type C.

TABLE 4-3
Major XRPD diffraction peaks of Compound 3
Hydrochloride Salt Type B

| Peak No. | 2θ Position [°] | Intensity [cts] |
|---|---|---|
| 1 | 11.753 | 409.1 |
| 2 | 12.295 | 679.9 |
| 3 | 16.851 | 572.6 |
| 4 | 22.331 | 455.0 |
| 5 | 23.055 | 241.4 |
| 6 | 23.565 | 295.9 |
| 7 | 25.329 | 820.0 |
| 8 | 27.496 | 563.1 |
| 9 | 28.054 | 284.2 |
| 10 | 30.059 | 408.4 |

Sulfate Salt Type A.

A manual salt screening was performed using Compound 3 Freebase Type A using approximately 100 mg of starting material and 15 μL of concentrated sulfuric acid were mixed in a 20-mL glass vial at a molar ratio of 1:1 in acetone. The resulted mixture was magnetically stirred at RT for 4 days. The resulting solids were isolated and dried at 40° C. for 4 hrs. Analysis by XRPD displayed the compound to be highly crystalline, designated as Compound 3 Sulfate Sate Type A. The major XRPD diffraction peaks are showed in Table 4-4. TGA result shows a weight loss of 0.68% up to 100° C. and 8.19%0, up to 200° C. and DSC curve shows two endotherm at 170.9° C. and 217.3° C. (peak) followed by possible melting at 226.4° C. (peak) and a endotherm at 275.3° C. Birefringent irregular shaped crystals were observed for the sulfate salt. ¹H-NMR results showed the NMR spectrum of the sulfate salt was similar to free base. DVS result shows a water uptake of 20.5% at 25° C./80% RH, indicating that Compound 3 Sulfate Sate Type A is highly hygroscopic. No significant form change was observed for the Compound 3 Sulfate Sate Type A after DVS, except for and additional peak at around 26=7°.

TABLE 4-4
Major XRPD diffraction peaks of
Compound 3 Sulfate Salt Type A

| Peak No. | 2θ Position [°] | Intensity [cts] |
|---|---|---|
| 1 | 5.225 | 1592.0 |
| 2 | 10.849 | 641.0 |
| 3 | 14.600 | 787.9 |
| 4 | 18.253 | 561.4 |
| 5 | 19.626 | 379.8 |
| 6 | 20.884 | 682.3 |
| 7 | 22.516 | 355.1 |
| 8 | 24.241 | 361.7 |
| 9 | 25.582 | 808.2 |
| 10 | 27.967 | 431.8 |

Glycolate Salt Types A and B.

Compound 3 Glycolate Salt Type A was obtained from slurry of the free base and counter ion in acetone. The XRPD pattern suggests it is crystalline. Major XRPD diffraction peaks are showed in Table 4-5. TGA result shows a weight loss of 10.6% up to 100° C. DSC curve shows two endotherm at 97.4° C. and 111.5° C. (peak) followed by possible solid-to-solid phase transition or recrystallization at 184.7° C. and a melting at 254.4° C. (peak), indicative of a potential solvate/hydrate.

TABLE 4-5
Major XRPD diffraction peaks of Compound 3
Glycolate Salt Type A

| Peak No. | 2θ Position [°] | Intensity [cts] |
|---|---|---|
| 1 | 6.814 | 1655.0 |
| 2 | 8.996 | 905.7 |
| 3 | 11.768 | 410.4 |
| 4 | 13.147 | 277.6 |
| 5 | 16.275 | 306.9 |
| 6 | 20.437 | 273.6 |
| 7 | 23.632 | 292.3 |
| 8 | 25.023 | 323.8 |
| 9 | 25.519 | 700.3 |
| 10 | 27.587 | 401.1 |

The scale up batch of glycolate salt was prepared by combining ˜100 mg of free base and glycolic acid into a 20.0-mL glass vial at a ratio of API:acid former around 1:1. After the addition of 2 mL of acetone into the vial, the suspension was stirred for two days at RT. The salt was isolated by centrifuge and vacuum drying at 40° C. for 4 hrs. The XRPD pattern of this material was shown to be different with the Compound 3 Glycolate Salt Type A, and assigned as Compound 3 Glycolate Salt Type B. Birefringent particles were observed for the Compound 3 Glycolate Salt Type B. TGA result shows a weight loss of 6.4% up to 100° C. and 11.1% up to 150° C. DSC curve shows one endotherm at 103.9° C. (peak) followed by possible exotherm at 132.6° C. and an endotherm at 231.9° C. (peak). ¹H-NMR results indicated by analysis of methylene protons at ˜3.9 ppm of glycolate suggested the molar ratio of API:acid former is 1:1. DVS of Compound 3 Glycolate Salt Type B showed around 45.3% water uptake from 0% to 80% RH. Compound 3 Glycolate Salt showed deliquescence post DVS experiment.

Succinate Salt Type A.

Compound 3 Succinate Type A was obtained from slurry of the free base and counter ion in acetone or EtOAc. The XRPD pattern suggests it is crystalline. Major XRPD diffraction peaks were showed in Table 4-6. TGA result shows a weight loss of 6.7% up to 100° C. DSC curve shows two endotherms, at 92.4° C. and 182.2° C. (peak). The first endotherm is likely related to solvent loss, suggestive of a hydrate/solvate.

TABLE 4-6
Major XRPD diffraction peaks of Compound 3
Succinate Salt Type A

| Peak No. | 2θ Position [°] | Intensity [cts] |
|---|---|---|
| 1 | 6.840 | 701.3 |
| 2 | 7.564 | 1151.5 |
| 3 | 8.975 | 276.8 |
| 4 | 11.769 | 348.8 |
| 5 | 14.788 | 240.9 |
| 6 | 22.052 | 238.0 |
| 7 | 23.312 | 335.4 |
| 8 | 25.688 | 481.9 |
| 9 | 27.322 | 252.4 |
| 10 | 32.741 | 452.4 |

### Example 5: Synthesis of Compound 4R ((R)-1-(2-methoxy-5-((4-methyl-6-(methylamino)-pyrimidin-2-yl)amino)phenoxy)-3-(pyrrolidin-1-yl)propan-2-ol)

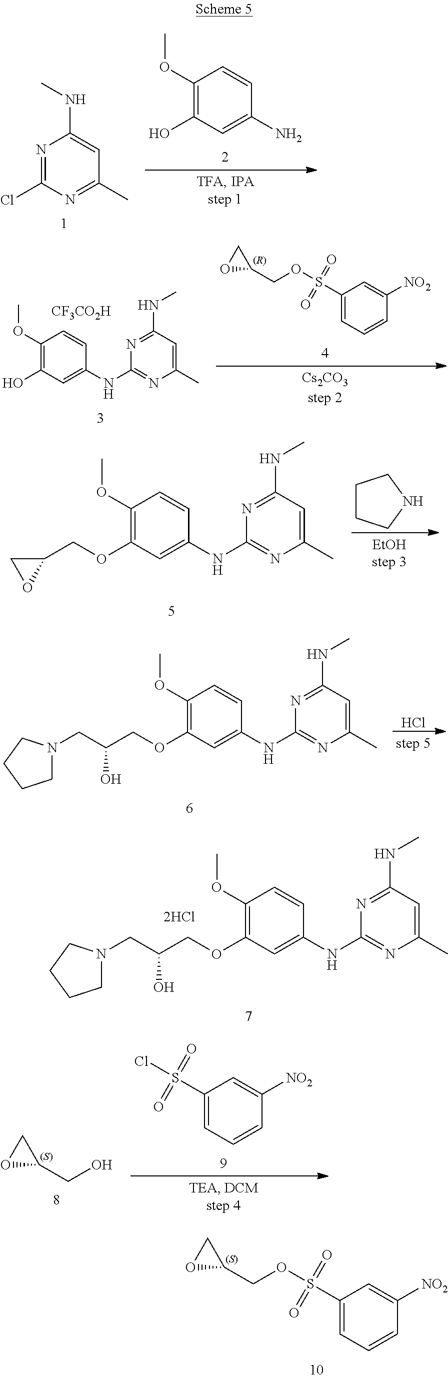

Step 1:

A 20 L reactor was charged with IPA (7.2 L, 8 v). 2-chloro-N,6-dimethylpyrimidin-4-amine (895.0, 1.0 eq.) and 5-amino-2-methoxyphenol (2) (720.0, 1.0 eq.) were added to the reactor under nitrogen. TFA (1180.0 g, 2.0 eq.) was added dropwise and the reaction mixture was stirred under hydrogen for 3 h at 80° C. The reaction was monitored by HPLC until 2 was present in ≤1%. The reaction was cooled to 0-10° C. The reaction mixture was filtered and the filter cake was washed with pre-cooled IPA (1.35 L, 1.5 v). The filter cake was dried to obtain 3 as a grey solid (1.5 Kg, purity: 99.7%, yield: 77.4%).

Step 2.

A 5 L 4-necked round bottom flask was charged with DMF (1.75 L, 5 v) under nitrogen. 3 (350.0 g, 1.0 eq.) was added to the round bottom flask followed by Cs₂CO₃ (914.7 g, 3.0 eq.). 4 (363.6 g, 1.5 eq.) was added to the reaction mixture. The reaction mixture was stirred for 4 h under hydrogen at 20-30° C. The reaction was monitored by HPLC until INTB-1 was present in ≤4%. Water (500 mL, 5 v) was added to the mixture and the solution was stirred for 0.5 h. The product was extracted with EA (500 mL, 5 v) four times and the organic phases were combined. The organic phase was washed with brine (500 mL, 5 v) three times. The organic phase was concentrated to 4-5 v. The resultant solution was charged with EtOH (500 mL, 5 v) and the solution was concentrate to 4-5 v. The round bottom flask was charged with EtOH (500 mL, 5 v) and the crude product was carried on to the next step.

Step 3.

The crude solution of step 2 was charge into a 5 L reactor. Pyrrolidine (265.8 g, 4.0 eq.) was added drop-wise at 20-30° C. The reaction was stirred for 4 h. The reaction was monitored until 5 was present in ≤2%. The reaction mixture was concentrated to 3-4 v. Twice, the resultant solution was charged with DCM (1750 mL, 5 v) and concentrated to 3-4 v. Twice, the organic phase was washed with water (1750 mL, 5 v). The aqueous phase was extracted with DCM (1050 mL, 3 v). The organic phases were combined and dried with Na2SO4. Twice, the organic phase was concentrated to 3-4 v. Twice, the resultant solution was charged with ACN (1750 mL, 5 v) and concentrated to 3-4 v. The reaction mixture was stirred for 3 h at 25-30° C. The reaction mixture was filtered and the filter cake was washed with ACN (350 mL, 1 v). MeOH (1400 mL, 2 v) was added to another reactor. The filter cake was charged to the reactor and heated to 70° C. The resultant reaction mixture was stirred for about 1 h. The reaction was cooled to 40° C. and charged with ACN (1400 mL, 2 v). The reaction was continued to cool to 0-10° C. and stirred for about 2 h. The reaction mixture was filtered and the filter cake was washed with ACN (700 mL, 1 v). The filter cake was dried in the oven at 40° C. for 16 h to obtain 537.0 g of final product as a light brown solid with purity: 99.0%, ee: 99.2% and 120.0 g with purity: 98.8%.

Freebase Type A.

Compound 4R Freebase Type A showed an XRPD pattern with low crystallinity by XRPD. Major XRPD diffraction peaks were showed in Table 5-1. The TGA curve showed 6.8% weight loss before 100° C. DSC curve displayed a broad endotherm around 77.8° C., followed by an endotherm at 229.2° C. and a possible exotherm at 240.7° C. indicating that freebase Type A is likely a solvate/hydrate. A reversible heat flow in mDSC curve displayed a possible melting endotherm around 219.2° C. (peak). No distinct morphology is was exhibited, though birefringence was observed for freebase Type A under PLM.

TABLE 5-1
Major XRPD diffraction peaks of
Compound 4R Freebase Type A

| Peak No. | 2θ Position [°] | Intensity [cts] |
|---|---|---|
| 1 | 6.401 | 427.8 |
| 2 | 7.165 | 269.0 |
| 3 | 9.855 | 25.0 |
| 4 | 13.309 | 180.7 |
| 5 | 15.709 | 37.9 |
| 6 | 26.104 | 389.2 |

Freebase Type B.

Compound 4R freebase Type B was produced from a slurry of freebase Type A in acetone. The sample was found to be crystalline by XRPD and assigned as freebase Type B. Major XRPD diffraction peaks are showed in Table 5-2. TGA result shows a weight loss of 2% up to 100° C. DSC curve shows a broad endotherm at 77.5° C. and a possible melting endotherm at 204.6° C. (peak), suggesting a likely solvate/hydrate form.

TABLE 5-2
Major XRPD diffraction peaks of
Compound 4R Freebase Type B

| Peak No. | 2θ Position [°] | Intensity [cts] |
|---|---|---|
| 1 | 6.313 | 1577.1 |
| 2 | 6.729 | 2325.7 |
| 3 | 9.244 | 1516.2 |
| 4 | 12.658 | 754.3 |
| 5 | 13.129 | 1252.5 |
| 6 | 14.374 | 669.9 |
| 7 | 20.078 | 760.6 |
| 8 | 21.997 | 632.2 |
| 9 | 26.152 | 972.5 |
| 10 | 27.049 | 470.0 |

Freebase Type C.

100 mg Slurry of Type A in acetone produced a new different XRPD pattern from the freebase Type B and was assigned as Compound 4R Freebase Type C. Major XRPD diffraction peaks of Compound 4R Freebase Type C are showed in Table 5-3. TGA result of Compound 4R Freebase Type C shows a weight loss of 4.4% up to 140° C. DSC curve shows a broad endotherm at 104.8° C. and a possible melting endotherm at 248.0° C. (peak) and is likely solvate/hydrate. Birefringent irregular shaped crystals were observed for the scale up Compound 4R Freebase Type C sample under PLM. DVS of Compound 4R Freebase Type C sample showed around 8.7% water uptake from 0% to 80% RH, indicating that Compound 4R Freebase Type C is hygroscopic. No form change was observed for Compound 4R Freebase Type C after DVS test as shown in XRPD overlay.

TABLE 5-3
Major XRPD diffraction peaks of
Compound 4R Freebase Type C

| Peak No. | 2θ Position [°] | Intensity [cts] |
|---|---|---|
| 1 | 7.260 | 296.1 |
| 2 | 7.959 | 125.0 |
| 3 | 8.799 | 118.7 |
| 4 | 9.819 | 151.7 |
| 5 | 12.397 | 123.7 |
| 6 | 13.311 | 258.8 |
| 7 | 26.182 | 501.1 |

Step 5.

A 20 L reactor was charged with MeOH (5300 mL, 10 v). 6 (530.0 g, 1.0 eq) was charged to the reactor. The reaction mixture was warmed to 45° C. and stirred for about 1 h until the solid dissolved. The mixture was cooled to 0-10° C. HCl/EA (3.0 eq, 1.0 mol/L) was added dropwise at 0-10° C. The reaction was stirred for 1 h at 0-10° C. MTBE (7950 mL, 15 v) was added to the solution. The reaction was stirred for 2 h at 0-10° C. The reaction was filtered and the filter cake was washed with MTBE (1590 mL, 3 v). The filter cake was dried in oven at 40-45° C. for 40 h to obtain 615.0 g of final product as off-white solid with purity: 99.0%, ee: 99.1%.

Hydrochloride Salt Type A.

Compound 4R Hydrochloride Salt Type A was found to be crystalline by XRPD. Major XRPD diffraction peaks were showed in Table 5-2. The TGA curve showed a 7.8% weight loss before 100° C. Its DSC curve displayed a broad endotherm around 75.7° C. followed by a possible melting endotherm at 198.7° C. (peak temp). The sample is likely a solvate/hydrate. Birefringent irregular shaped crystals were observed for Compound 4R Hydrochloride Salt Type A under PLM. DVS of Compound 4R Hydrochloride Salt Type A sample showed around 4% water uptake from 50% RH to 80% RH at 25° C., indicating that Compound 4R Hydrochloride Salt Type A is hygroscopic. As shown by XRPD, change of XRPD pattern was observed for Compound 4R Hydrochloride Salt Type A before and after DVS.

TABLE 5-2
Major XRPD diffraction peaks of Compound 4R
Hydrochloride Salt Type A

| Peak No. | 2θ Position [°] | Intensity [cts] |
|---|---|---|
| 1 | 6.342 | 1254.8 |
| 2 | 11.800 | 460.3 |
| 3 | 14.498 | 756.8 |
| 4 | 15.511 | 345.0 |
| 5 | 19.355 | 255.8 |
| 6 | 25.503 | 1322.3 |
| 7 | 26.277 | 667.6 |
| 8 | 29.381 | 276.0 |

Hydrochloride Salt Type B.

Compound 4R Hydrochloride Salt Type B to 100 mg was successfully achieved in acetone by weighing ˜100 mg of Compound 4R Freebase Type A into a 20.0-mL glass vial and adding 2 mL of acetone into a second vial with 42 μL concentrated hydrochloric acid. The diluted acid solution into the first vial at a ratio of API:acid former around 1:1. The suspension was stirred for two days at RT before isolating the solid by centrifuge and vacuum drying at 40° C. for 4 hrs. Compound 4R Hydrochloride Salt Type B batch showed an XRPD pattern consistent with the sample being crystalline. Major XRPD diffraction peaks are showed in Table 5-3. TGA result shows a weight loss of 3.4%/o up to 150° C. DSC curve shows a possible melting endotherm at 239.2° C. (peak) in addition to a broad endotherm at ˜100° C. corresponding to weight loss. Birefringent irregular shaped crystals were observed for Compound 4R Hydrochloride Salt Type B under PLM. DVS of Compound 4R Hydrochloride Salt Type B sample showed around 17.4% water uptake from 0% to 80% RH, indicating that Compound 4R Hydrochloride Salt Type B is very hygroscopic. No form change was observed after DVS test as shown in XRPD overlay.

TABLE 5-3
Major XRPD diffraction peaks of Compound 4R
Hydrochloride Salt Type B

| Peak No. | 2θ Position [°] | Intensity [cts] |
|---|---|---|
| 1 | 7.198 | 1121.9 |
| 2 | 7.946 | 402.2 |
| 3 | 8.766 | 353.5 |
| 4 | 9.780 | 540.4 |
| 5 | 12.374 | 549.6 |
| 6 | 13.258 | 616.5 |
| 7 | 14.412 | 123.7 |
| 8 | 17.597 | 156.7 |
| 9 | 26.222 | 341.8 |

Succinate Salt Type A.

Compound 4R Succinate Salt Type A was successfully prepared in 100 mg scale by weighing ˜100 mg of Compound 4R Freebase Type A and 61 mg of acid into a 20.0-mL glass vial at a ratio of API:acid former around 1:1. 2 mL of acetone is added into the vial and the suspension stirred for two days at RT. The solat was isolated by centrifuge and vacuum drying at 40° C. for 4 hrs. The scale up batch showed an XRPD consistent with a crystalline solid. Major XRPD diffraction peaks of succinate salt hit are showed in Table 5-4. TGA result shows a weight loss of 2.00 up to 120° C. DSC curve shows multiple endotherm at 88.7° C., 147.0° C., 193.6° C. (peak) followed by a exotherm peak at 232.0° C. (peak). Birefringent particles were observed for the succinate salt. DVS of succinate salt showed around 9.6% water uptake from 0% to 80% RH, indicating that succinate salt is hygroscopic. No form change was observed post DVS testing.

TABLE 5-3
Major XRPD diffraction peaks of
Compound 4R Succinate Salt Type A

| Peak No. | 2θ Position [°] | Intensity [cts] |
|---|---|---|
| 1 | 6.313 | 1577.1 |
| 2 | 6.7285 | 2325.7 |
| 3 | 9.244 | 1516.2 |
| 4 | 12.658 | 754.3 |
| 5 | 13.129 | 1252.5 |
| 6 | 14.374 | 669.9 |
| 7 | 20.078 | 760.6 |
| 8 | 21.997 | 632.2 |
| 9 | 26.152 | 972.5 |
| 10 | 27.049 | 470.0 |

### Synthesis of Intermediate

Step 4:

A 5 L 4-necked round bottom was charged with DCM (2 L, 10 v) under nitrogen. The solution was cooled to −20 to −30° C. TEA (409.5 g, 1.5 eq.) was added to the reaction under nitrogen. A solution of DCM (2 L, 10 v) and 3-nitrobenzene-1-sulfonyl chloride (200.0 g, 1.0 eq) was added dropwise. The reaction was stirred for 1 h under hydrogen at −10 to −20° C. The reaction was monitored by HPLC until 3-NsCl was present in ≤2%. The reaction mixture was filtered and washed with DCM (400 mL, 2 v). The filtrate was collected and washed twice with water (1 L, 5 v). The organic phase was collected and concentrated to 3-4 v/w. Twice, the solution was charged with MTBE (1 L, 5 v) and concentrate to 3-4 v. i-PrOH (200 mL, 1 v) and water (3 L, 15 v) were added to the solution and stirred for 3 h. The mixture was filtered and the filter cake was washed with water (400 ml, 2 v). The filter cake was dried in an oven at 40° C. for 16 h to obtain 520.0 g product as an off-white solid with purity: 97.8%, yield: 72.7%.

### Example 6: Synthesis of Compound 4S ((S)-1-(2-methoxy-5-((4-methyl-6-(methylamino)-pyrimidin-2-yl)amino)phenoxy)-3-(pyrrolidin-1-yl)propan-2-ol)

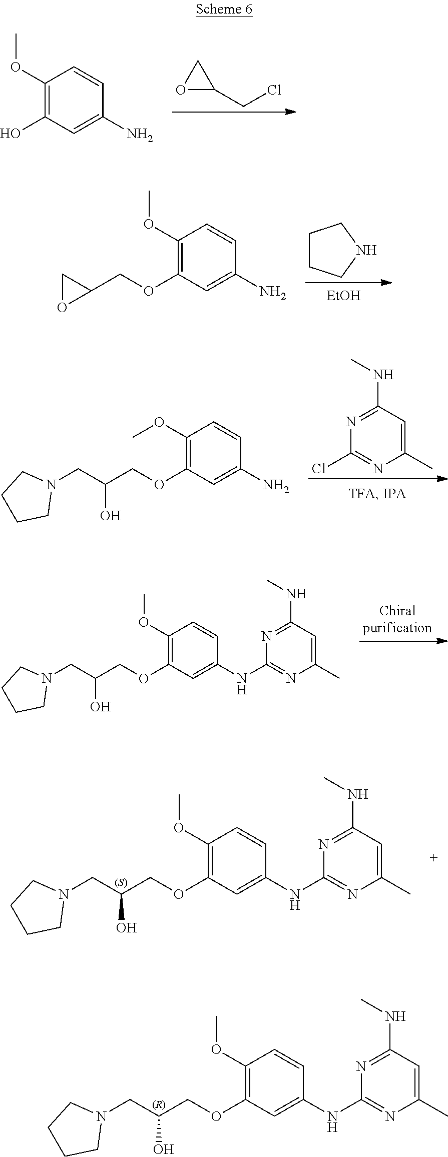

Compound 4S was synthesized according to the Scheme 6 illustrated above.

### Example 7: Synthesis of Compound 5R ((R)-1-(azetidin-1-yl)-(2-methoxy-5-((4-methyl-6-(methylamino)pyrimidin-2-yl)amino)phenoxy)propan-2-ol)

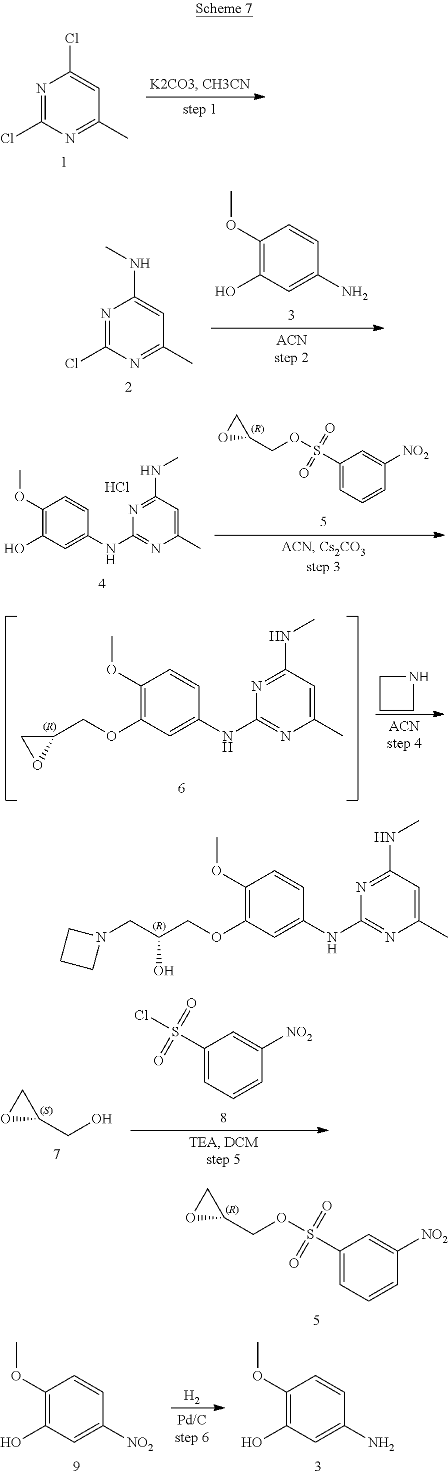

Step 1.

A 50 L flask was charged with 2,4-dichloro-6-methylpyrimidine (2.0 kg, 1.0 eq.) and acetonitrile (40.0 L, 20.0 v/w. Anhydrous K₂CO₃ powder (5.1 kg, 3.0 eq.) was added into the 50 L reactor. The mixture was cooled to 0-5° C. Methanamine hydrochloride (992.4 g, 1.2 eq.) was added into the mixture at 0-5° C. The reaction mixture was stirred for at least 12 hours at 0-25° C. The reaction was monitored with HPLC until 2,4-dichloro-6-methylpyrimidine was present in less than 1.0%. The reaction was then combined with an additional 2 kg. batch of material. The reaction was filtered and the organic phase was concentrated under vacuum to 2-3 v at 30-40° C. The filter cake was washed with DCM (20.0 L, 5.0 v/w). DCM (44.0 L, 11.0 v/w) was added to the filtrate. The DCM organic phase was washed with 15% brine (20.0 L, 5.0 v/w). The organic phase was concentrated under vacuum to 3-4 v at 30-40° C. Toluene (20.0 L, 5.0 v/w) was added to the solution and the mixture was concentrated to about 5 v/w. Toluene (20.0 L, 5.0 v/w) was added to the solution and the mixture was concentrated to about 8 v. Toluene (16.0 L, 4.0 v/w, total 12 v/w) was charged into the 50 L reactor. The reaction mixture was heated to 60-65° C. and stirred until the mixture was dissolved completely. The solution was cooled to 35° C. over 3 hours and the solid precipitated out at about 36° C. The mixture was stirred for about 3 hours at 35-38° C. The solution was cooled to 28-33° C. over about 2 hours. The mixture was stirred for about 2 hours at 28-33° C. The reaction was filtered and the filter cake was washed with toluene (12 L, 3.0 v/w). The crude filter cake was combined with another two 2 kg batches. The reactor, under nitrogen atmosphere, was charged with MTBE (19.4 L, 6.0 v/w). Crude 2 (3.24 kg, 1.0 eq) was added to the reactor. The reaction mixture was heated to 50-55° C. and stirred for 16 hours. The reaction mixture was cooled slowly to 10-15° C. with an average hourly cooling of 10-15° C. The reaction was kept at 10-15° C. and stirred for at least 4 hours. The reaction was filtered and the filter cake was washed with MTBE (3.2 L, 1.0 v/w) twice. The filter cake was dried under vacuum at 50-55° C. for 16 hours until the LOD was not more than 1.0%. The product was obtained as an off-white solid (3.0 kg), with 99.90% purity, Yield: 38.8%.

Step 2.

To a 5000 ml reactor was charged ACN (3000 ml, 10 v/w) and 2 (300.0 g, 1.0 eq.). 3 (264.9 g, 1.0 eq.) was added to the reactor. The reaction was heated to 70-75° C. The reaction mixture was stirred for 16 h at 70-75° C. The reaction was monitored until 3 was present in ≤1%. The reaction was cooled to 10-15° C. and stirred for about 2 h. The reaction was filtered and the filter cake was washed with ACN (450 ml, 1.5 v/w) twice. The filter cake was dried in the oven under vacuum at 40-45° C. for at least 16 h until the LOD <1.0%. The product was obtained as a light brown sold (480 g, purity: 98.3%, yield: 93.5%, LOD=0.89%. Q-NMR=101%).

Steps 3 and 4.

A 5 L reactor was charged with ACN (1.6 L, 8 v/w). 4 (200 g, 1.0 eq.) was added to the reaction followed by Cs₂CO₃ (549.1 g, 2.5 eq.). The reaction was stirred for 0.5 h. 5 (192.2 g, 1.1 eq.) was added to the reaction. The reaction was heated to 30-35° C. and stirred for 4 h. The reaction was monitored until 4 was present in ≤1.5%. The reaction mixture was filtered and the filter cake was washed with ACN (300 ml, 1.5 v/w) twice. Activated carbon (160 g, 0.8 w/w) was added and the reaction mixture stirred for 16 h at 15-20° C. The reaction mixture was filtered and the filter cake was washed with ACN (160 ml, 0.8 v/w) twice. Azetidine (130.6 g, 4.0 eq.) was added and the reaction mixture was heated to 30-40° C. The reaction was stirred for 16 h. The reaction was monitored until 6 was present in ≤1.5%. The reaction mixture was concentrated to 4-6 v. The reaction was first cooled to 15-20° C., then cooled to 0-5° C. The reaction was filtered and the filter cake was washed with ACN (75 ml, 0.5 v/w) twice. The filter cake was dried in an oven under vacuum at 35-45° C. for at least 16 h to obtain the crude product 173.2 g as a light brown solid (purity: 96.1%, ee: 99.1%, assay by HPLC: 94.0%).

Freebase Type A.

Compound 5R freebase was found to be crystalline by XRPD and assigned as freebase Type A (Table 6-1). TGA curve showed 1.4% weight loss before 150° C., and DSC curve displayed a desolvation endotherm at 104.3° C. (onset) followed by an crystallization exotherm at 115.7 (peak) and a second melting endotherm at 137.9° C. (onset). Birefringent irregular shape crystals were observed for Type A under PLM. The freebase Type B was prepared by heating Type A to 130° C. and then cooled down to RT.

TABLE 6-1
Major XRPD diffraction peaks of Compound 5R Freebase Type A

| Peak No. | 2θ Position [°] | Intensity [cts] |
|---|---|---|
| 1 | 12.812 | 2028.5 |
| 2 | 13.387 | 2623.6 |
| 3 | 14.574 | 993.1 |
| 4 | 17.550 | 1373.1 |
| 5 | 20.851 | 1550.7 |
| 6 | 23.913 | 1019.6 |

Freebase Type B.

Compound 5R Freebase Type B can be obtained via slurry of Type A in various solvents (water, EtOAc and acetone) at RT or by heating Type A to 130° C. and cooling down to RT. Compound 5R freebase Type B was found to be crystalline by XRPD (Table 6-2). Scale up of Type B to 100 mg was successfully achieved by slurry of Type A in acetone. The scale up of Type B batch showed the same XRPD as the initial hit. TGA curve showed 0.3% weight loss before 150° C., and DSC curve displayed one melting endotherm at 138.0° C. (onset).

TABLE 6-2
Major XRPD diffraction peaks of Compound 5R Freebase Type B

| Peak No. | 2θ Position [°] | Intensity [cts] |
|---|---|---|
| 1 | 10.168 | 1004.9 |
| 2 | 12.487 | 1534.4 |
| 3 | 13.965 | 1690.6 |
| 4 | 17.748 | 2326.5 |
| 5 | 18.818 | 2072.7 |
| 6 | 19.340 | 1350.4 |
| 7 | 24.555 | 1112.6 |

Freebase Type C.

Compound 5R Freebase Type C can be obtained via crystallization of Compound 5R from a mixture of MeOH—H2O and EtOH—H2O. Major XRPD diffraction peaks of Compound 5R Freebase Type C are showed in Table 6-3.

TABLE 6-3
Major XRPD diffraction peaks of Compound 5R Freebase Type C

| Peak No. | 2θ Position [°] | Intensity [cts] |
|---|---|---|
| 1 | 8.475 | 769 |
| 2 | 12.861 | 1080 |
| 3 | 13.554 | 749 |
| 4 | 15.408 | 712 |
| 5 | 16.012 | 404 |
| 6 | 18.139 | 631 |
| 7 | 21.317 | 498 |
| 8 | 21.632 | 805 |
| 9 | 22.866 | 397 |
| 10 | 24.836 | 357 |

Preparing Crystalline Salts of Compound 5R.

Compound 5R Freebase Type A was used as starting material for salt screening. A manual salt screening was performed under 22 conditions using 11 pharmaceutically acceptable counter ions in two solvent systems. About 20 mg of starting material and corresponding counter ion were mixed into each 2-mL glass vial at a molar ratio of 1:1. Control experiments were conducted in the same solvents without counter ion. 0.3 mL of corresponding solvent was then added to form a suspension or clear solution. The resulted mixture was magnetically stirred at RT for 4 days. Solids isolated were analyzed by XRPD to determine crystallinity. In Table 6-4, weight loss values were determined by TGA assessment and thermal event peaks are derived from DSC study.

TABLE 6-4

| | | | | Thermal |
|---|---|---|---|---|
| | | Possible | | Events |
| Crystalline | Solvent of | Solid | Weight | (° C., |
| Form | preparation | Form | Loss (%) | peak) |
| Compound 5R | acetone | Hydrate/ | 8.3 | 123.5 |
| Sulfate | | solvate | | |
| Salt Type A | | | | |
| Compound 5R | acetone | Hydrate/ | 10.6 | 58.7, 225.2 |
| Glycolate | | solvate | | |
| Salt Type A | | | | |
| Compound 5R | acetone | Hydrate/ | 6.9 | 92.4, 182.2 |
| Fumarate | | solvate | | |
| Salt Type A | | | | |
| Compound 5R | acetone | Hydrate/ | 3.7 | 99.8 |
| Hippurate | | solvate | | |
| Salt Type A | | | | |
| Compound 5R | acetone | Hydrate/ | 4.1 | 58.0 |
| Adipate | | solvate | | |
| Salt Type A | | | | |
| Compound 5R | methanol | Hydrate/ | 2.4 | 179 |
| Gentisate | | solvate | | |
| Salt Type A | | | | |
| Compound 5R | THF/water | Hydrate/ | 1.6 | 156 |
| Gentisate | | solvate | | |
| Salt Type E | | | | |
| Compound 5R | methanol | Hydrate | 6.2 | 179 |
| Ethanedi- | | | | |
| sulfonate | | | | |
| Salt Type A | | | | |
| Compound 5R | THF/water | Hydrate | 2.8 | 163 |
| Benezene- | | | | |
| sulfonate | | | | |
| Salt Type A | | | | |
| Compound 5R | methanol | Hydrate | 0.4 | 171 |
| Benzoate | | | | |
| Salt Type A | | | | |
| Compound 5R | HFIPA | Hydrate/ | 7.89, 14.95 | 161 |
| Benzoate | | solvate | | |
| Salt Type B | | | | |
| Compound 5R | HFIPA | Hydrate/ | 8.77, 25.63 | 164 |
| Benzoate | | solvate | | |
| Salt Type C | | | | |
| Compound 5R | Dioxane/ | Hydrate/ | 4.24 | 73, 162 |
| Benzoate | water | solvate | | |
| Salt Type E | | | | |
| Compound 5R | THF/water, | Hydrate/ | 4.73, 3.46 | 93, 163 |
| Benzoate | dioxane/ | solvate | | |
| Salt Type F | water | | | |

Sulfate Salt Type A.

Compound 5R Sulfate Salt Type A was obtained from slurry of the free base with counter ion in acetone or EtOAc. The XRPD pattern displayed suggests it is crystalline. The major XRPD diffraction peaks of Compound 5R Sulfate Salt Type A were listed in Table 6-5.

TABLE 6-5
Major XRPD diffraction peaks of
Compound 5R Sulfate Salt Type A.

| Peak No. | 2θ Position [°] | Intensity [cts] |
|---|---|---|
| 1 | 6.766 | 648.0 |
| 2 | 8.652 | 1621.6 |
| 3 | 13.946 | 1068.7 |
| 4 | 16.417 | 773.0 |
| 5 | 23.494 | 471.2 |
| 6 | 25.287 | 689.6 |
| 7 | 26.49772 | 743.9492 |

Glycolate Salt Type A.

Compound 5R Glycolate Salt Type A was obtained from slurry of the free base and counter ion in acetone or EtOAc. The XRPD pattern displayed suggests it is crystalline. The major XRPD diffraction peaks of Compound 5R Glycolate Salt Type A were listed in Table 6-6.

TABLE 6-6
Major XRPD diffraction peaks of Compound
5R Glycolate Salt Type A.

| Peak No. | 2θ Position [°] | Intensity [cts] |
|---|---|---|
| 1 | 6.519 | 911.4 |
| 2 | 14.059 | 326.9 |
| 3 | 17.830 | 540.5 |
| 4 | 18.938 | 497.2 |
| 5 | 24.686 | 400.4 |
| 6 | 25.672 | 445.3 |
| 7 | 26.488 | 460.4 |

Fumarate Salt Type A.

Compound 5R Fumarate Salt Type A was obtained from slurry of the free base and counter ion in acetone or EtOAc. The XRPD pattern displayed suggest it is crystalline. The major XRPD diffraction peaks were listed in Table 6-7. Compound 5R Fumarate Salt Type A scale-up was successfully prepared by weighing ˜100 mg of freebase and 31 mg of fumaric acid into a 20.0-mL glass vial at a ratio of API:acid, around 1:1. Add 2 mL of acetone into the vial and stir the suspension for two days at RT. Isolate solid by centrifuge and vacuum drying at 40° C. for 4 hrs. The scale up batch showed the same XRPD pattern with the initial hit. Birefringent particles were observed for Compound 5R Fumarate Salt Type A. ¹H-NMR results indicated an alkenyl proton of fumarate at 6.6 ppm suggested the molar ratio of API:acid former was 1:1. DVS result shows a water uptake of 7.6% at 25° C./80% RH, indicating that Compound 5R Fumarate Salt Type A is hygroscopic. No significant form change was observed for fumarate salt after DVS.

TABLE 6-7
Major XRPD diffraction peaks of
Compound 5R Fumarate Salt Type A

| Peak No. | 2θ Position [°] | Intensity [cts] |
|---|---|---|
| 1 | 5.942 | 2222.4 |
| 2 | 7.660 | 1631.9 |
| 3 | 11.306 | 536.1 |
| 4 | 11.879 | 572.4 |
| 5 | 15.397 | 771.1 |
| 6 | 18.405 | 706.3 |
| 7 | 25.839 | 730.0 |
| 8 | 26.470 | 1673.1 |

Hippurate Salt Type A.

Compound 5R Hippurate Salt Type A was obtained from slurry of the free base and counter ion in acetone and EtOAc. The XRPD pattern displayed suggests it is crystalline. The major XRPD diffraction peaks were listed in Table 6-8. Compound 5R Hippurate Salt Type A scale-up was successfully prepared by weighing ˜100 mg of freebase and 48 mg of hippuric acid into a 20.0-mL glass vial at a ratio of API:acid, around 1:1. Add 2 mL of acetone into the vial and stir the suspension for two days at RT. Isolate solid by centrifuge and vacuum drying at 40° C. for 4 hrs. The scale up batch showed the same XRPD pattern as the initial hit. Birefringent particles were observed for the hippurate salt. ¹H-NMR results indicated phenyl protons at 7.8 and 7.5 ppm suggested the molar ratio of API:acid former is 1:1. DVS result shows a water uptake of 2.6% at 25° C./80% RH, indicating that Compound 5R Hippurate Salt Type A is hygroscopic. No significant form change was observed for hippurate salt after DVS.

TABLE 6-8
Major XRPD diffraction peaks of Compound
5R Hippurate Salt Type A.

| Peak No. | 2θ Position [°] | Intensity [cts] |
|---|---|---|
| 1 | 6.492 | 2262.9 |
| 2 | 9.695 | 777.4 |
| 3 | 10.975 | 506.7 |
| 4 | 12.963 | 3473.9 |
| 5 | 19.443 | 904.5 |
| 6 | 23.623 | 818.0 |
| 7 | 26.073 | 1027.8 |

Adipate Salt Type A.

Compound 5R Adipate Salt Type A was obtained from slurry of the free base and counter ion in various acetone and EtOAc. The XRPD pattern is displayed suggests it is crystalline. The major XRPD diffraction peaks were listed in Table 6-9.

TABLE 6-9
Major XRPD diffraction peaks of
Compound 5R Adipate Salt Type A.

| Peak No. | 2θ Position [°] | Intensity [cts] |
|---|---|---|
| 1 | 10.655 | 577.3 |
| 2 | 13.064 | 640.4 |
| 3 | 17.775 | 590.4 |
| 4 | 18.841 | 524.0 |
| 5 | 21.548 | 1604.5 |
| 6 | 22.894 | 649.3 |
| 7 | 24.546 | 508.6 |
| 8 | 25.448 | 2346.8 |

Gentisate Salt Type A.

40 mg of Compound 5R was combined with 1:1 or 1:2 molar equivalents of gentisic acid in 40 volumes of methanol. After stirring at room temperature for 2 hours, crystalline material was isolated after slow evaporation of the solvent and filtration. XRPD suggested a crystalline material, assigned Compound 5R Gentisate Salt Type A. Major XRPD peaks of Compound 5R Gentisate Salt Type A are listed in Table 6-10.

TABLE 6-10
Major XRPD diffraction peaks of
Compound 5R Gentisate Salt Type A

| Peak No. | 2θ Position [°] | Intensity [cts] |
|---|---|---|
| 1 | 5.252 | 373 |
| 2 | 7.655 | 241 |
| 3 | 8.836 | 243 |
| 4 | 9.335 | 246 |
| 5 | 14.968 | 206 |
| 6 | 16.216 | 232 |
| 7 | 17.148 | 213 |
| 8 | 21.245 | 171 |
| 9 | 25.262 | 339 |

Gentisate Salt Type E.

40 mg of Compound 5R was combined with 1:3 molar equivalents of gentisic acid in 40 volumes of THF/water (75:25, % v/v). After stirring at room temperature for 2 hours, the crystalline material was isolated after slow evaporation of the solvent and filtration. XRPD suggested a crystalline material, assigned Compound 5R Gentisate Salt Type E. Major XRPD peaks of assigned Compound 5R Gentisate Salt Type A are listed in Table 6-11. TGA results shows a weight loss of 0.4% between RT and 150° C. DSC curve shows a melt/decomposition occurring at 179° C. ¹HNMR suggests a digentisate salt. No form change was observed after stressing at 40° C./75% RH.

TABLE 6-11
Major XRPD diffraction peaks of Compound
5R Gentisate Salt Type E.

| Peak No. | 2θ Position [°] | Intensity [cts] |
|---|---|---|
| 1 | 6.013 | 500 |
| 2 | 9.125 | 289 |
| 3 | 15.021 | 296 |
| 4 | 17.739 | 243 |
| 5 | 18.408 | 306 |
| 6 | 20.719 | 267 |
| 7 | 23.766 | 206 |
| 8 | 25.840 | 222 |
| 9 | 26.615 | 262 |

Benzoate Salt Type A.

Compound 5R Benzoate Salt Type A has been prepared on a small scale using MeOH and MTBE. The salt precipitated when a methanolic solution of the free base was combined with a stock solution of benzoic acid in MeOH. Yield was ˜54%. Further precipitation was achieved by addition of MTBE anti-solvent to the supernatant. XRPD analysis of the solids showed the material was crystalline. Major peaks of from the XRPD data of Compound 5R Benzoate Salt Type A are shown in Table 6-12. The Compound 5R Benzoate Salt Type A has also been prepared on a gram scale using MeOH. The salt precipitated when a methanolic solution of the freebase was combined with a stock solution of benzoic acid in MeOH. Yield was ˜75%. The material was dried under vacuum.

TABLE 6-12
Major XRPD diffraction peaks of
Compound 5R Benzoate Salt Type A.

| Peak No. | 2θ Position [°] | Intensity [cts] |
|---|---|---|
| 1 | 5.278 | 1465 |
| 2 | 9.664 | 451 |
| 3 | 15.507 | 729 |
| 4 | 18.251 | 388 |
| 5 | 19.029 | 706 |
| 6 | 21.271 | 500 |
| 7 | 22.912 | 441 |
| 8 | 23.726 | 440 |
| 9 | 26.930 | 784 |

Benzoate Salt Type B.

Compound 5R Benzoate Dalt type B material was prepared four times from HFIPA evaporation experiments. The XRPD pattern of Compound 5R Benzoate Dalt type B shows a disordered crystalline solid with the Major XRPD peaks listed in Table 6-13.

TABLE 6-13
Major XRPD diffraction peaks of
Compound 5R Benzoate Salt Type B

| Peak No. | 2θ Position [°] | Intensity [cts] |
|---|---|---|
| 1 | 7.904 | 290 |
| 2 | 10.084 | 266 |
| 3 | 11.712 | 280 |
| 4 | 17.187 | 441 |
| 5 | 24.435 | 657 |
| 6 | 25.131 | 312 |

Benzoate Salt Type C.

Compound 5R Benzoate Salt Type C was prepared five times using HFIPA as a solvent with different anti-solvents from evaporation and vapour diffusion experiments. The XRPD pattern indicates that the material is crystalline. Major XRPD peaks of Compound 5R Benzoate Salt Type C are listed in Table 6-14.

TABLE 6-14
Major XRPD diffraction peaks of
Compound 5R Benzoate Salt Type C.

| Peak No. | 2θ Position [°] | Intensity [cts] |
|---|---|---|
| 1 | 5.514 | 449 |
| 2 | 11.095 | 454 |
| 3 | 14.325 | 413 |
| 4 | 15.927 | 394 |
| 5 | 16.741 | 322 |
| 6 | 17.043 | 327 |
| 7 | 17.450 | 335 |
| 8 | 19.144 | 380 |
| 9 | 24.435 | 648 |
| 10 | 24.855 | 387 |

Benzoate Salt Type E.

Compound 5R Benzoate Salt Type E was prepared using dioxane/water (1:1) from freeze drying experiments. The XRPD pattern of Compound 5R Benzoate Salt Type E shows a crystalline form with major XRPD peaks shown in in Table 6-15.

TABLE 6-15
Major XRPD diffraction peaks of
Compound 5R Benzoate Salt Type E.

| Peak No. | 2θ Position [°] | Intensity [cts] |
|---|---|---|
| 1 | 5.685 | 340 |
| 2 | 6.250 | 341 |
| 3 | 12.565 | 263 |
| 4 | 15.362 | 213 |
| 5 | 25.105 | 276 |

Benzoate Salt Type F.

Compound 5R Benzoate Salt Type F was prepared three times, twice using THF/water (1:3) from salt formation experiments and once from freeze drying. The XRPD of Compound 5R Benzoate Salt Type F shows a crystalline form and the major XRPD peaks are listed in Table 6-16.

TABLE 6-16
Major XRPD diffraction peaks of
Compound 5R Benzoate Salt Type F.

| Peak No. | 2θ Position [°] | Intensity [cts] |
|---|---|---|
| 1 | 6.079 | 5081 |
| 2 | 12.290 | 1401 |
| 3 | 16.268 | 971 |
| 4 | 18.343 | 848 |
| 5 | 21.218 | 604 |
| 6 | 22.164 | 517 |
| 7 | 23.096 | 562 |
| 8 | 24.409 | 1313 |
| 9 | 26.247 | 664 |

### Synthesis of Intermediates

Step 5. A 1000 ml four neck reactor was charged with DCM (250 ml, 5 v/w), 7 (50 g, 1.0 eq.). The mixture was cooled to 0±5° C. and charged with TEA (102 g, 1.5 eq.). A solution of DCM (250 ml, 5 v/w) and 8 (165 g, 1.1 eq.) was added dropwise to the 1000 ml reactor at 0±5° C. The reaction was stirred for 2 h and analyzed by ¹H-NMR. The reaction mixture was filtered and the filter cake was washed with DCM (50 ml, 1 v/w) twice. Water (250 ml, 5 v/w) was added to the reaction and the mixture was separated. The organic phases was collected. The organic phase was washed with water (250 ml, 5 v/w). The water phases were combined and extracted with DCM (75 ml, 1.5 v/w). The organic phase was separated and concentrated to 2-3 v. The resultant solution was charged with MTBE (250 ml, 5 v/w) and concentrated to 2-3 v twice. MTBE (400 ml, 8 v/w) was added and the mixture was stirred at 0-10° C. for 8 h. The reaction was filtered and the filter cake was washed with MTBE (100 ml, 2 v/w) twice. The filter cake was dried in the oven at 35-40° C. for 16 h to obtain 147.3 g product as light yellow solid with purity: 98.3%, yield: 82.8%.

Step 6.

A 5000 ml hydrogenation reactor was charged with MeOH (6.0 L, 7.5 v/w) and 9 (800.0 g, 1.0 eq.). DCM (6.0 L, 7.5 v/w) was added to the reactor followed by wet Pd/C (40.0 g, 5.0% w/w.). The reactor was filled with hydrogen at 5-10 atm at 20-30° C. Hydrogen was added two additional times at 20 atm at 20-30° C. The reaction was stirred for 6 h at 20-30° C. The reaction was monitored until 9 was present in ≤1%. The reaction was filtered and the filter cake was washed with MeOH (400 ml, 0.5 v/w) twice. The organic phase was concentrated to 0.5-1 v. DCM (4.0 L, 5 v/w) was added to the resultant solution and the mixture was concentrated to 1-2 v twice. The sample was analyzed by GC. The reaction mixture was stirred for 1 h at 15-25° C. The reaction was filtered and the filter cake was washed with DCM (400 ml, 0.5 v/w) twice. The filter cake was dried in an oven under vacuum at 35-40° C. for at least 12 h to obtain the 488.5 g brown solid (purity: 99.5%, yield: 74.2%, Q-NMR: 98.2%).

### Example 8: Synthesis of Compound 5S ((S)-1-(azetidin-1-yl)-3-(2-methoxy-5-((4-methyl-6-(methylamino)pyrimidin-2-yl)amino)phenoxy)propan-2-ol)

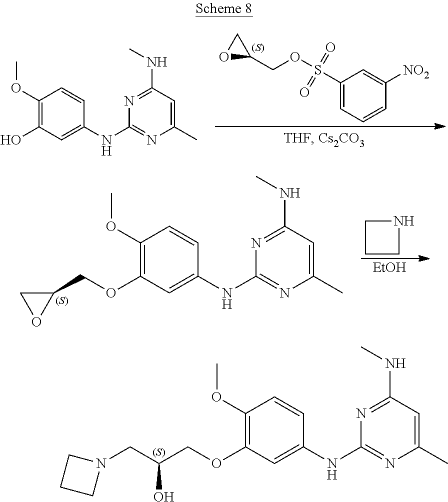

Compound 5S was synthesized according to the Scheme 8 illustrated above.

### Example 9: Synthesis of Compound 6 (N2-(4-methoxy-3-(4-((methylamino)methyl)-1H-pyrazol-1-yl)phenyl)-N4,6-dimethylpyrimidine-2,4-diamine)

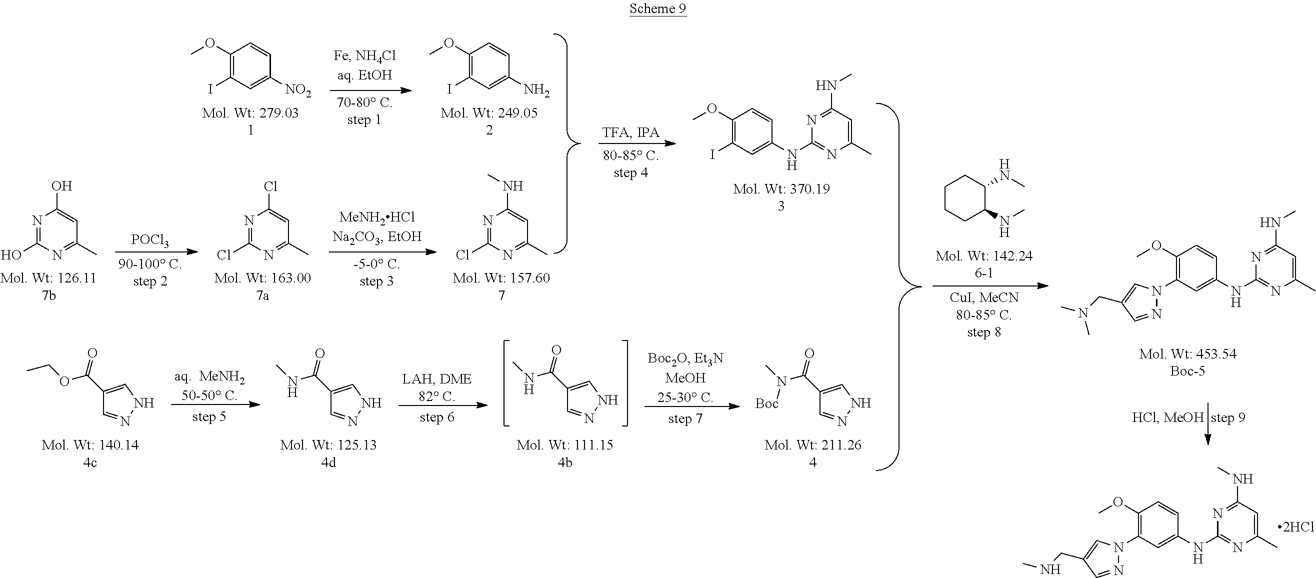

Step 1.

Fe (490 g, 8.78 mol, 3.5 eq.) and NH4Cl (684 g, 12.8 mol, 5.1 eq.) were charged into EtOH (10 L, 14 vol.) and water (4.2 L, 6 vol.) in a flask (20 L) under a N2 atmosphere. The mixture was heated to 70-80° C. Nitroarene 1 (700 g, 2.51 mol, 1.0 eq.) was added into the mixture in portions (gas was released). The mixture was stirred at 70-80° C. for 1 h. The reaction was monitored by HPLC until 1 was consumed completely. The mixture was cooled to room temperature and filtered with the aid of diatomite (210 g, 0.3 w/w). The filter cake was washed with EtOH (300 mL×4). The filtrates were combined and the majority of EtOH was removed by distillation. EtOAc (7.0 L, 10.0 vol.) and water (3.5 L, 5.0 vol.) were added to the residue. The mixture was stirred for 30 min and the organic layer was separated. The aqueous layer was extracted with EtOAc (7.0 L, 10.0 vol.) and the organic layer was separated. The organic layers were combined and dried over anhydrous Na2SO4 (250 g). The mixture was filtered and the filter cake was washed with EtOAc (500 mL). The filter solutions were combined and concentrated to dryness. 609 g of aniline 2 as a dark brown solid was afforded with 98.1 A % HPLC purity in 95% isolated yield.

Step 2:

Pyrimidine 7b (1.0 kg, 7.9 mol, 1.0 eq.) and POCl₃ (8.0 L, 8.0 vol.) were charged into a flask (20 L) under a N2 atmosphere. The mixture was stirred and heated to 90-100° C. The reaction mixture turned to a clear solution after 2 h. The reaction proceeded at 90-100° C. for about 8 h. The reaction was monitored by HPLC until 7b was consumed to below 0.1%. (HPLC showed 7b consumed completely; 7a was 97.0 A % and impurity 7a-1 was 2.3 A %). The reaction was combined with an additional batch of the reaction solution for workup. The reaction mixture was concentrated to remove the majority of POCl₃. DCM (10.0 L, 10.0 vol.) was added to the residue. The resulting solution was added dropwise slowly into 25% of aq. K₂CO₃ at <40° C. The pH of the aqueous layer was 3-4. The organic phase was separated and the aqueous phase was extracted with DCM (10.0 L, 10.0 vol.). The combined organic phase was dried over anhydrous Na2SO4 (˜200 g). The mixture was filtered and the filter cake was washed with DCM (500 mL×2). The filtrate was evaporated to dryness at 40° C. under reduced pressure. The solid was dried at 40° C. to afford yellow 7a (2.1 kg) with 99.4% HPLC purity in 90% isolated yield.

Step 3:

Chloropyrimidine 7a (2.1 kg, 13.1 mol, 1.0 eq.), Na2CO₃ (3.6 kg, 34.1 mol, 2.6 eq.) and EtOH (40 L, 20.0 vol.) were charged into a flask (60 L) under a N2 atmosphere. The mixture was cooled to −5-0° C. MeNH₂.HCl (972 g, 14.4 mol, 1.1 eq.) was added into the mixture. The mixture was stirred at −5-0° C. for 45 h. The reaction was monitored by HPLC. The solid was filtered and the filter cake was washed with EtOH (500 mL×4). The filtrates were combined and concentrated to dryness. 2.2 kg of crude 7 was obtained with 70%/HPLC. Crude 7 was added into PhMe and the suspension was stirred at room temperature for 2 h. The reaction was filtered and the filter cake was washed with PhMe (500 mL). The filter cake was dried under high vacuum at 60° C. for 3 h. 800 Grams of 7 as a yellow solid was obtained with 98.1 A % HPLC purity in 40% corrected yield based on 7a.

Step 4:

To a solution of aniline 2 (609 g, 2.44 mol, 1.0 eq.) and chloropyrimidine 7 (385 g, 2.44 mol, 1.0 eq.) in IPA (6 L, 10 vol.) was added TFA (278 g, 2.44 mol, 1.0 eq.) under a N2 atmosphere. The solution was heated at 80-85° C. for 4 h and gradually turned into a suspension. The reaction was monitored by HPLC (HPLC showed unreacted 2 and 7 were 0.6 A % and 0.9 A %). The suspension was evaporated to remove the majority of the IPA. EtOAc (3.6 L, 6.0 vol.) was added to the residue and slurried for 30 min at room temperature. The reaction mixture was filtered and the filter cake was washed with EtOAc (500 mL). The filter cake was added into EtOAc (12 L, 20.0 vol.) and water (3 L, 5.0 vol.). Sat. aq. NaHCO₃ (3 L, 5.0 vol.) was added to adjust the pH value of the aqueous phase to 7-8. The mixture was stirred for 30 min and the organic layer was separated. The aqueous layer was extracted with EtOAc (6 L, 10.0 vol.) and the organic layer was separated. The combined organic layer was dried over anhydrous Na2SO4 (500 g). The mixture was filtered and the filter cake was washed with EtOAc (500 mL). The filtrate was evaporated to dryness. 814 g of 3 as a dark brown solid was afforded with 97 A % HPLC purity in 90% yield.

Step 5:

Ester 4c (695 g, 4.96 mol, 1.0 eq.) was added into 40 wt % MeNH₂ in water (3.5 L, 5 vol.) under a N2 atmosphere. The suspension was heated to 50-55° C. for 3-4 h and gradually dissolved. The reaction was monitored by HPLC (HPLC showed 4c was consumed completely by HPLC). Water was removed by distillation at 65° C. PhMe (3.5 L×2) was added into the solid and the water was removed by distillation at 65° C. 620 g of 4d was obtained as an off-white solid with 97 A % HPLC purity in quantity yield.

Step 6:

Amide 4d (620 g, 4.95 mol, 1.0 eq.) was added into DME (12 L, 20 vol.) under a N2 atmosphere. The suspension was cooled to −10° C. LAH (471 g, 12.39 mol, 2.5 eq.) was added into the suspension in portions at −10-10° C. The mixture was heated to 82° C. with stirring for 14-18 h. The reaction was monitored by HPLC (HPLC showed 4d was remained 14.4 A %). The suspension was cooled to 25-40° C. The suspension was added into 20 wt % aq. NaOH (7.5 L, 12 vol.) at −10-40° C. The organic layer was separated and the aqueous layer was extracted with DME (6 L, 10 vol.) The organic layers (containing aq. NaOH) were combined and used directly in the next step without further purification.

Step 7:

Boc₂O (864 g, 3.96 mol, 0.8 eq.) was added into the combined organic layers of step 6 (containing DME and aq. NaOH) at 25-30° C. The solution was stirred for 1-2 h. The reaction was monitored by. The mixture was extracted with EtOAc (10 L×2). The organic layers were combined and dried with anhydrous Na2SO4 (1 kg). The mixture was filtered and the filter cake was wash with EtOAc (1 L.) The filtrates were combined and concentrated by distillation. The residue was purified by silica gel column chromatography with PE/EtOAc (v/v: 10/on and MeOH. 430 g of 4 as a semi-solid was obtained with 94.5 A % HPLC purity in 41% isolated yield.

Step 8.

Aryl iodide 3 (250 g, 0.675 mol, 1.0 eq.), pyrazole 4 (171 g, 0.81 mol, 1.2 eq.), K₂CO₃ (186.6 g, 1.35 mol, 2.0 eq.) were added to MeCN (3.5 L, 15 vol.). The mixture was backfilled with Ar four times. Ar was bubbled through the mixture for 2 h at 25-30° C. CuI (25.7 g, 0.135 mol, 0.2 eq.) and ligand 6-1 (76.8 g, 0.54 mol, 0.8 eq.) were added quickly into the mixture. The mixture was backfilled with argon four times. The reaction was heated to 80-85° C. with stirring for 14-20 h. The reaction was monitored by HPLC (HPLC showed 3 was consumed completely). The reaction was cooled to room temperature. The mixture was filter through diatomite and the filter cake was washed with EtOAc (1 L×2). The filter solutions were combined with another three batches of Boc-5 and concentrated to remove solvents. The residue was purified by silica gel column chromatography (n-heptane/EtOAc; v/v: 5-1/1). 744 g of Boc-5 was produced with 99.2 A % HPLC purity in 69% isolated yield.

Step 9.

To Boc-5 (269 g, 0.59 mol, 1.0 eq.) was added MeOH (2.7 L, 10 vol.) at 25-30° C. (another batch using 269 g of Boc-5 was conducted in parallel). 10 M HCl in MeOH (350 mL, 3.56 mol, 6.0 eq.) was added into the solution at 25-30° C. over a period of 30 min. The solution was stirred for 2 h at 25-30° C. and solids precipitated gradually. The reaction was monitored by HPLC (unreacted Boc-5 was 1.5 A % by HPLC). EtOAc (11 L, 40 vol.) was added dropwise into the suspension at 25-30° C. for 1 h. The suspension was cooled to 0-5° C. and stirred for 1 h at 0-5° C. The mixture was combined with another batch and filtered. The cake was washed with EtOAc (1 L×2). The wet product was dried under high vacuum (10-20 mmHg) at 60° C. for 6-8 h. 475 g of hydrochloride salt of Compound 6 was afforded from the two batches with 99.5 A % HPLC purity in 93% isolated yield.

Freebase Type A.

Compound 6 freebase Type A was prepared by suspending 100 mg of amorphous free base in 2 mL acetone in a 20-mL glass vial and stirring for 3 days at 800 RPM at RT. Solid Freebase Type A was isolated from the suspension via centrifuging and drying. The sample was found to be crystalline by XRPD and the major XRPD diffraction peaks are showed in Table 7-1. TGA result shows a weight loss of 0.24% up to 200° C. The DSC curve showed an endotherm at 217.8° C. (peak) likely due to melting. Birefringent irregular shaped crystals were observed for free base Type A under PLM. DVS of Type A sample showed around 4.4% water uptake from 0% to 80% RH, indicating that Type A is hygroscopic. No form change was observed after DVS test as shown by XRPD.

TABLE 7-1
Major XRPD diffraction peaks of
Compound 6 Freebase Type A

| Peak No. | 2θ Position [°] | Intensity [cts] |
|---|---|---|
| 1 | 4.500 | 883.9 |
| 2 | 9.674 | 624.0 |
| 3 | 10.465 | 693.4 |
| 4 | 13.492 | 1445.7 |
| 5 | 15.311 | 485.7 |
| 6 | 18.052 | 623.7 |
| 7 | 24.329 | 439.0 |
| 8 | 25.766 | 1699.5 |

Hydrochloride Salt Type A.

Compound 6 Hydrochloride Salt Type A showed a crystalline pattern by XRPD. Major XRPD diffraction peaks of Compound 6 Hydrochloride Salt Type A are showed in Table 7-2. TGA result shows a weight loss of 8.7% up to 150° C. DSC curve shows a broad endotherm at 159.5° C. (likely desolvation/dehydration), followed by an endotherm at 207.3° C. and possible a recrystallization exotherm at 216.9° C. and a second endotherm at 278.1° C. (peak) with decomposition. Birefringent irregular shaped crystals were observed for Compound 6 Hydrochloride Salt Type A under PLM. DVS of Compound 6 Hydrochloride Salt Type A showed around 19.4% water uptake from 0% to 80% RH, indicating that Compound 6 Hydrochloride Salt Type A is very hygroscopic. Form change was observed for Compound 6 Hydrochloride Salt Type A post DVS.

TABLE 7-2
Major XRPD diffraction peaks of
Compound 6 Hydrochloride Salt Type A

| Peak No. | 2θ Position [°] | Intensity [cts] |
|---|---|---|
| 1 | 5.242 | 658.6 |
| 2 | 9.853 | 1920.6 |
| 3 | 10.749 | 2920.0 |
| 4 | 11.483 | 908.7 |
| 5 | 19.673 | 548.3 |
| 6 | 21.482 | 642.3 |
| 7 | 24.094 | 770.1 |
| 8 | 25.129 | 5885.2 |
| 9 | 27.047 | 1071.3 |
| 10 | 27.615 | 1081.6 |

Glycolate Salt Type A.

Compound 6 Glycolate Salt Type A was obtained from slurry of the free base with counter ion in EtOAc or acetone. The XRPD pattern suggests it is crystalline. Major XRPD diffraction peaks were showed in Table 7-3. Sample of Compound 6 Glycolate Salt Type A was appeared to be a wet solid under ambient condition due to high hygroscopicity, therefore it was not tested by TGA/DSC.

TABLE 7-3
Major XRPD diffraction peaks of
Compound 6 Glycolate Salt Type A

| Peak No. | 2θ Position [°] | Intensity [cts] |
|---|---|---|
| 1 | 5.707 | 657.0 |
| 2 | 7.044 | 671.2 |
| 3 | 10.247 | 436.3 |
| 4 | 15.124 | 151.8 |
| 5 | 16.073 | 141.5 |
| 6 | 21.638 | 216.9 |
| 7 | 25.794 | 467.0 |
| 8 | 27.683 | 449.8 |

Adipate Salt Type A.

Compound 6 Adipate Salt Type A was obtained from slurry of the free base and counter ion in acetone. The XRPD pattern displayed suggested it was crystalline and major XRPD diffraction peaks were showed in Table 7-4. TGA result shows a weight loss of 8.6% up to 120° C. DSC curve shows one broad endotherm at 96.5° C. (likely desolvation/dehydration) followed by possible recrystallization exotherm at 172.2° C. and a possible melting at 195.2° C. (peak), indicative of a potential solvate/hydrate.

TABLE 7-4
Major XRPD diffraction peaks of
Compound 6 Adipate Salt Type A

| Peak No. | 2θ Position [°] | Intensity [cts] |
|---|---|---|
| 1 | 5.823 | 314.6 |
| 2 | 7.758 | 2019.2 |
| 3 | 10.506 | 350.7 |
| 4 | 11.257 | 488.0 |
| 5 | 14.348 | 346.4 |
| 6 | 24.632 | 287.6 |
| 7 | 25.593 | 719.6 |
| 8 | 26.282 | 762.5 |

Adipate Salt Type B.

During an attempted scale-up of Compound 6 Adipate Salt Type A, salt product showed a different XRPD pattern from the Compound 6 Adipate Salt Type A sample and was assigned as Compound 6 Adipate Salt Type B. The procedure used required weighing ˜100 mg of freebase compound 6 and the corresponding adipic acid into a 20.0-mL glass vial at a ratio of API:acid former around 1:1. Then, 2 mL of acetone was added into the vial and the suspension stirred for two days at RT. The solids was isolated by centrifuge and vacuum drying at 40° C. for 4 hrs. Major XRPD diffraction peaks were showed in Table 7-5. Birefringent particles were observed for the Compound 6 Adipate Salt Type B. TGA result shows a weight loss of 0.7% up to 150° C. DSC curve shows two endotherms at 159.5 and 191.9° C. (peak). DVS of Compound 6 Adipate Salt Type B showed around 8.8% water uptake from 0% to 80% RH, indicating that adipate salt is hygroscopic. A form change from Compound 6 Adipate Salt Type B to Compound 6 Adipate Salt Type A was observed post DVS as evidenced by XRPD overlay of adipate salt before and after DVS. ¹H-NMR results indicated by integration of a methoxyl group of the API at ˜3.8 ppm and methylene protons of adpate at ˜1.5 ppm suggested the molar ratio of API:acid former is 2:1.

TABLE 7-5
Major XRPD diffraction peaks of
Compound 6 Adipate Salt Type B

| Peak No. | 2θ Position [°] | Intensity [cts] |
|---|---|---|
| 1 | 5.283 | 288.2 |
| 2 | 5.957 | 380.3 |
| 3 | 8.114 | 459.2 |
| 4 | 11.594 | 466.7 |
| 5 | 11.909 | 388.0 |
| 6 | 14.732 | 185.7 |
| 7 | 21.580 | 171.5 |
| 8 | 24.004 | 245.2 |
| 9 | 25.531 | 590.5 |
| 10 | 26.364 | 190.1 |

### Example 10: Synthesis of Compound 7 (N2-(2-fluoro-4-methoxy-3-(4-((methylamino)methyl)-1H-pyrazol-1-yl)phenyl)-N4,6-dimethylpyrimidine-2,4-diamine hydrochloride)

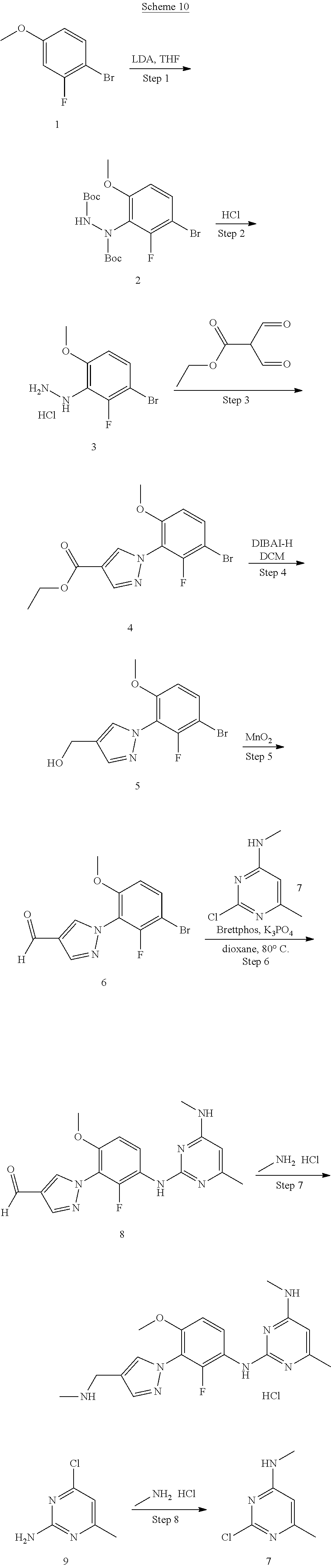

### Step 1. Synthesis of N-(3-bromo-2-fluoro-6-methoxyphenyl)-N-(tert-butoxy)carbonyl]butoxy)carbohydrazide

Into the solution of 1-bromo-2-fluoro-4-methoxybenzene (1000 g, 4.88 mol, 1.00 equiv) in tetrahydrofuran (10 L) was added dropwise LDA (2561 mL, 1.05 equiv) at −78° C. under nitrogen. The resulting solution was stirred for 1 h at −70° C. Then to the above solution, (Z)-N-[(tert-butoxy)carbonyl]imino(tert-butoxy)formamide (1122 g, 4.87 mmol, 1.00 equiv.) was added. The resulting solution was stirred for 1 h at −78° C. The reaction was then quenched by the addition of 200 mL of methanol. The resulting mixture was concentrated under vacuum and dissolved in EA (6 L), washed with water (2 L) for two times and organic layers concentrated under vacuum. This resulted in 1400 g (66%) of the title compound as a white solid.

### Step 2: Synthesis of 3-bromo-2-fluoro-6-methoxyphenyl)hydrazine

Into the solution of N-(3-bromo-2-fluoro-6-methoxyphenyl)-N-(tert-butoxy)carbonyl](tert-butoxy)carbohydrazide (1400 g, 3.22 mol, 1.00 equiv) in ethanol (10 L) was added hydrogen chloride (3000 mL). The resulting solution was stirred for 2 h at room temperature. The resulting mixture was concentrated under vacuum. This resulted in 958 g (crude) of the title compound as a white solid.

### Step 3: Synthesis of ethyl 1-(3-bromo-2-fluoro-6-methoxyphenyl)-1H-pyrazole-4-carboxylate

Into the solution of (3-bromo-2-fluoro-6-methoxyphenyl)hydrazine (958 g, 4.08 mol, 1.00 equiv) in ethanol (10 L) was ethyl 2-formyl-3-oxopropanoate (558 g, 3.87 mol, 0.95 equiv.). The resulting solution was stirred for 2 h at room temperature. The resulting mixture was concentrated under vacuum. The residue was stirred with PE (5000 mL) for 0.5 h. The solids were collected by filtration. This resulted in 958 g of the title compound as a white solid.

### Step 4: Synthesis of [1-(3-bromo-2-fluoro-6-methoxyphenyl)-1H-pyrazol-4-yl]methanol

Into the solution of ethyl 1-(3-bromo-2-fluoro-6-methoxyphenyl)-1H-pyrazole-4-carboxylate (950 g, 2.77 mol, 1.00 equiv) in dichloromethane (8000 mL) was added dropwise DIBAL-H (4167 mL, 3.00 equiv, 2 mol/L) at 0° C. under nitrogen. The resulting solution was stirred for 2 h at room temperature. The reaction was then quenched by the addition of 3000 mL of water. The resulting solution was extracted with 3×3000 mL of ethyl acetate and the organic layers combined and dried over anhydrous sodium sulfate and concentrated under vacuum. This resulted in 494 g (55%) of the title compound as a yellow solid.

### Step 5: Synthesis of 1-3-bromo-2-fluoro-6-methoxyphenyl)-1H-pyrazole-4-carbaldehyde

Into the solution of [1-(3-bromo-2-fluoro-6-methoxyphenyl)-1H-pyrazol-4-yl]methanol (470 g, 1.56 mol, 1.00 equiv.) in ethyl acetate (12 L) was added dioxomanganese (2045 g, 23.52 mol, 15.00 equiv.). The resulting solution was stirred overnight at room temperature. The solids were filtered out. The resulting mixture was concentrated under vacuum. This resulted in 379 g (81%) of the title compound as a brown solid.

### Step 6: Synthesis of 1-(2-fluoro-6-methoxy-3-[[4-methyl-6-(methylamino)pyrimidin-2-yl]amino]phenyl)-1H-pyrazole-4-carbaldehyde

Into the solution of 1-(3-bromo-2-fluoro-6-methoxyphenyl)-1H-pyrazole-4-carbaldehyde (379 g, 1.27 mol, 1.00 equiv) in dioxane (4000 mL) was added N-4,6-dimethylpyrimidine-2,4-diamine (177 g, 1.28 mol, 1.00 equiv.), K₃PO₄ (404 g, 1.91 mol, 1.50 equiv.) and Brettphos Pd (38 g, 41.94 mmol, 0.03 equiv). The resulting solution was stirred for 16 h at 100° C. in an oil bath. The resulting mixture was concentrated under vacuum. The resulting mixture was washed with 3×1000 mL of EA and water. The organic layer was separated and concentrated. The residue was applied onto a silica gel column with ethyl acetate/hexane (2/1). This resulted in 298 g (66%) of the title compound as a brown solid.

### Step 7: Synthesis of N2-(2-fluoro-4-methoxy-3-(4-((methylamino)methyl)-1H-pyrazol-1-yl)phenyl)-N4,6-dimethylpyrimidine-2,4-diamine hydrochloride

Into a solution of 1-(2-fluoro-6-methoxy-3-[[4-methyl-6-(methylamino)pyrimidin-2-yl]amino]phenyl)-1H-pyrazole-4-carbaldehyde (218 g, 611.74 mmol, 1.00 equiv.) in THF (2000 mL), was added methylamine hydrochloride (1530 mL, 5.00 equiv), the resulting solution was stirred for 1 hour and then added NaBH(OAc)3 (389 g, 1.36 mol, 3.00 equiv). The resulting solution was stirred for 2 h at room temperature. The resulting mixture was concentrated under vacuum. The residue was applied onto a silica gel column with chloroform/methanol (10/l). the resulted solid was dissolved in methanol (100 ml) and was added HCl (50 ml), the solids were collected by filtration. This resulted in 79.2 g (61%) of the title compound. Analytical Data: LC-MS: (ES, m/z): [M+1]=372; ¹H NMR (300 MHz, Methanol-d4): δ 8.12 (d, J=4.1 Hz, 1H), 7.96 (s, 1H), 7.82 (dt, 1H), 7.16 (dd, 1H), 6.28-6.05 (m, 1H), 4.25 (s, 2H), 3.88 (d, 3H), 2.95 (d, 3H), 2.76 (s, 3H), 2.47-2.23 (m, 3H).

Hydrochloride Salt Type A.

Compound 7 Hydrochloride Salt Type A was found to be crystalline by XRPD. Major XRPD diffraction peaks are showed in Table 8-1. TGA curve showed 6.5% weight loss up to 180° C. DSC curve displayed endotherms around 77.3° C., 128.2, 170.2° C., and 210.6° C. and a possible melting endotherm at 231.7° C. (peak). The sample is likely a solvate/hydrate. Birefringent and needle shaped crystals were observed for Compound 7 Hydrochloride Salt Type A under PLM.

TABLE 8-1
Major XRPD diffraction peaks of
Compound 7 Hydrochloride Salt Type A.

| Peak No. | 2θ Position [°] | Intensity [cts] |
|---|---|---|
| 1 | 6.8 | 1160 |
| 2 | 9.4 | 270 |
| 3 | 12.1 | 360 |
| 4 | 14.5 | 510 |
| 5 | 15.0 | 435 |
| 6 | 18.7 | 300 |
| 7 | 24.2 | 360 |
| 8 | 25.1 | 405 |
| 9 | 25.6 | 890 |
| 10 | 26.8 | 525 |

Hydrochloride Salt Type B.

Compound 7 Hydrochloride Salt Type B was found to be crystalline by XRPD. Major XRPD diffraction peaks are showed in Table 8-2. TGA curve showed 8.5% weight loss up to 180° C. DSC curve displayed an endotherm around 87.8° C., another at 118.6° C. and a possible melting endotherm at 208.7° C. (peak). The sample is likely a solvate/hydrate. Birefringent and needle shaped crystals were observed for Compound 7 Hydrochloride Salt Type B under PLM.

TABLE 8-2
Major XRPD diffraction peaks of
Compound 7 Hydrochloride Salt Type B.

| Peak No. | 2θ Position [°] | Intensity [cts] |
|---|---|---|
| 1 | 5.9 | 590 |
| 2 | 8.3 | 875 |
| 3 | 10.0 | 995 |
| 4 | 11.7 | 275 |
| 5 | 21.9 | 300 |
| 6 | 25.1 | 1180 |
| 7 | 26.9 | 290 |

Oxalate Salt Type A (1:

2). 29.6 mg of Compound 7 freebase and 1.62 mL 0.1 mol/L Oxalic methanol solution was added into a 4 mL vial with stirring at room temperature. Then evaporated the solution to dry and added 1 mL MTBE stirred overnight, and the product was collected by filtration. Compound 7 Oxalate Salt Type A was found to be crystalline by XRPD. Major XRPD diffraction peaks are showed in Table 8-3. TGA curve showed 3.86% weight loss up to 150° C. DSC curve displayed an endotherm around 144.18° C., another at 211.24° C. and a possible melting endotherm at 208.7° C. (peak). The sample is likely a solvate/hydrate. Birefringent and irregular shaped crystals were observed for Compound 7 Oxalate Salt Type A under PLM.

TABLE 8-3
Major XRPD diffraction peaks of
Compound 7 Oxalate Salt Type A.

| Peak No. | 2θ Position [°] | Intensity [cts] |
|---|---|---|
| 1 | 4.5 | 760 |
| 2 | 8.7 | 880 |
| 3 | 9.1 | 1000 |
| 4 | 9.7 | 400 |
| 5 | 13.8 | 660 |
| 6 | 24.9 | 360 |
| 7 | 25.4 | 400 |

Sulfate Salt Type A (1:

2). 29.8 mg of Compound 7 freebase and 1.62 mL 0.1 mol/L H2SO4 methanol solution was added into a 4 mL vial with stirring at room temperature. Then evaporated the solution to dry and added 1 mL MTBE stirred overnight, and the product was collected by filtration. Compound 7 Sulfate Salt Type A was found to be crystalline by XRPD. Major XRPD diffraction peaks are showed in Table 8-4. TGA curve showed 2.05% weight loss up to 150° C. DSC curve displayed an endotherm around 113.35° C. and 152.06° C. and a possible melting endotherm at 185.31° C. (peak). The sample is likely a solvate/hydrate. Birefringent and irregular shaped crystals were observed for HCl salt Type B under PLM.

TABLE 8-4
Major XRPD diffraction peaks of
Compound 7 Sulfate Salt Type A.

| Peak No. | 2θ Position [°] | Intensity [cts] |
|---|---|---|
| 1 | 13.1 | 400 |
| 2 | 15.8 | 740 |
| 3 | 17.9 | 600 |
| 4 | 18.0 | 720 |
| 5 | 18.9 | 540 |
| 6 | 19.2 | 700 |
| 7 | 19.7 | 680 |
| 8 | 23.8 | 460 |
| 9 | 25.1 | 920 |
| 10 | 25.7 | 440 |
| 11 | 26.4 | 1020 |

Phosphate Salt Type A (1:2).

30.08 mg of Compound 7 freebase and 1.62 mL 0.1 mol/L H₃PO₄ methanol solution was added into a 4 mL vial with stirring at room temperature. Then evaporated the solution to dry and added 1 mL MTBE stirred overnight the product was collected by filtration. Compound 7 Phosphate Salt Type A was found to be crystalline by XRPD. Major XRPD diffraction peaks are showed in Table 8-5. TGA curve showed 3.44% weight loss up to 150° C. DSC curve displayed a broad endotherm around 76.7° C., another at 109.97° C., 140.25° C., and 183.80° C. and a possible melting endotherm at 209.41° C. (peak). The sample is likely a solvate/hydrate. Birefringent and irregular shaped crystals were observed for Compound 7 Phosphate Salt Type A under PLM.

TABLE 8-5
Major XRPD diffraction peaks of
Compound 7 Phosphate Salt Type A.

| Peak No. | 2θ Position [°] | Intensity [cts] |
|---|---|---|
| 1 | 13.8 | 340 |
| 2 | 14.4 | 500 |
| 3 | 15.3 | 320 |
| 4 | 16.8 | 220 |
| 5 | 24.1 | 380 |
| 6 | 25.0 | 620 |

Fumarate Salt Type A (1:2).

29.5 mg of Compound 7 freebase and 1.62 mL 0.1 mol/L fumaric methanol solution was added into a 4 mL vial with stirring at room temperature. Then evaporated the solution to dry and added 1 mL MTBE stirred overnight, and the product was collected by filtration. Compound 7 Fumarate Salt Type A was found to be crystalline by XRPD. Major XRPD diffraction peaks are showed in Table 8-6. TGA curve showed 0.35% weight loss up to 150° C. DSC curve displayed a possible melting endotherm at 230.99° C. (peak). The sample has low hygroscopicity. Birefringent and irregular shaped crystals were observed for Compound 7 Fumarate Salt Type A under PLM.

TABLE 8-6
Major XRPD diffraction peaks of
Compound 7 Fumarate Salt Type A.

| Peak No. | 2θ Position [°] | Intensity [cts] |
|---|---|---|
| 1 | 8.2 | 740 |
| 2 | 9.0 | 840 |
| 3 | 11.6 | 1400 |
| 4 | 14.4 | 720 |
| 5 | 16.6 | 1080 |
| 6 | 20.7 | 880 |
| 7 | 21.1 | 1440 |
| 8 | 22.2 | 880 |
| 9 | 24.5 | 1780 |

Fumarate Salt Type B (1:1).

30.17 mg of Compound 7 freebase and 0.81 mL 0.1 mol/L fumaric methanol solution was added into a 4 mL vial with stirring at room temperature. Then evaporated the solution to dry and added 1 mL MTBE stirred overnight, and the product was collected by filtration. Compound 7 Fumarate Salt Type B was found to be crystalline by XRPD. Major XRPD diffraction peaks are showed in Table 8-7. TGA curve showed 4.96% weight loss up to 150° C. DSC curve displayed a broad endotherm around 66.28° C., and another at 125.87° C. The sample is likely a solvate/hydrate. Birefringent and irregular shaped crystals were observed for Compound 7 Fumarate Salt Type B under PLM.

TABLE 8-7
Major XRPD diffraction peaks of
Compound 7 Fumarate Salt Type B.

| Peak No. | 2θ Position [°] | Intensity [cts] |
|---|---|---|
| 1 | 4.4 | 480 |
| 2 | 7.5 | 600 |
| 3 | 9.0 | 380 |
| 4 | 11.7 | 520 |
| 5 | 14.5 | 316 |
| 6 | 16.7 | 400 |
| 7 | 21.3 | 400 |
| 8 | 22.2 | 320 |
| 9 | 24.7 | 440 |
| 10 | 25.9 | 340 |

Fumarate Salt Type C (1:1.5).

Compound 7 Fumarate Salt Type C was found to be crystalline by XRPD. Major XRPD diffraction peaks are showed in Table 8-8. TGA curve showed 0.58% weight loss up to 150° C. DSC curve displayed a possible melting endotherm at 211.52° C. (peak). The sample has low hygroscopicity, DVS of Compound 7 Fumarate Salt Type C sample showed around 1.9% water uptake from 0% to 80% RH. Birefringent and irregular shaped crystals were observed for Compound 7 Fumarate Salt Type C under PLM.

TABLE 8-8
Major XRPD diffraction peaks of
Compound 7 Fumarate Salt Type C.

| Peak No. | 2θ Position [°] | Intensity [cts] |
|---|---|---|
| 1 | 9.7 | 1840 |
| 2 | 12.2 | 805 |
| 3 | 12.8 | 604 |
| 4 | 13.6 | 834 |
| 5 | 14.0 | 604 |
| 6 | 22.5 | 474 |
| 7 | 24.4 | 503 |
| 8 | 24.9 | 1107 |

### Synthesis of Intermediate

### Step 8: Synthesis of N-4,6-dimethylpyrimidine-2,4-diamine

Into the solution of 4-chloro-6-methylpyrimidin-2-amine (600 g. 4.18 mol, 1.00 equiv.) in NMP (6000 mL) was added potassium carbonate (1158 g, 8.38 mol, 2.00 equiv.) and methylamine hydrochloride (843 g, 12.49 mol, 3.00 equiv). The resulting solution was stirred for 36 h at 120° C. in a closed reactor. The resulting mixture was concentrated under vacuum and washed with 1×1000 mL of water. The solids were collected by filtration. This resulted in 211 g (37%) of the title compound as a white solid.

### Example 11: Bioactivity Assays

### Materials and Equipment

Recombinant purified human EHMT2 913-1193 (55 μM) synthesized by Viva was used for all experiments. Biotinylated histone peptides were synthesized by Biopeptide and HPLC-purified to >95% purity. Streptavidin Flashplates and seals were purchased from PerkinElmer and 384 Well V-bottom Polypropylene Plates were from Greiner. ³H-labeled S-adenosylmethionine (³H-SAM) was obtained from American Radiolabeled Chemicals with a specific activity of 80 Ci/mmol. Unlabeled SAM and S-adenosylhomocysteine (SAH) were obtained from American Radiolabeled Chemicals and Sigma-Aldrich respectively. Flashplates were washed in a Biotek ELx-405 with 0.1% Tween. 384-well Flashplates and 96-well filter binding plates were read on a TopCount microplate reader (PerkinElmer). Compound serial dilutions were performed on a Freedom EVO (Tecan) and spotted into assay plates using a Thermo Scientific Matrix PlateMate (Thermo Scientific). Reagent cocktails were added by Multidrop Combi (Thermo Scientific).

MDA-MB-231 cell line was purchased from ATCC (Manassas, Va., USA). RPMI/Glutamax medium, Penicillin-Streptomycin, Heat Inactivated Fetal Bovine Serum, and D-PBS were purchased from Life Technologies (Grand Island, N.Y., USA). Odyssey blocking buffer, 800CW goat anti-mouse IgG (H+L) antibody, and Licor Odyssey Infrared Scanner were purchased from Licor Biosciences, Lincoln, Nebr., USA. H3K9me2 mouse monoclonal antibody (Cat #1220) was purchased from Abcam (Cambridge, Mass., USA). 16% Paraformaldehyde was purchased from Electron Microscopy Sciences, Hatfield, Pa., USA). MDA-MB-231 cells were maintained in complete growth medium (RPMI supplemented with 10% v/v heat inactivated fetal bovine serum) and cultured at 37° C. under 5% CO₂ UNC0638 was purchased from Sigma-Aldrich (St. Louis, Mo., USA).

### General Procedure for FJHMT2 Enzyme Assay on Histone Peptide Substrate.

10-Point curves of test compounds were made on a Freedom EVO (Tecan) using serial 3-fold dilutions in DMSO, beginning at 2.5 mM (final top concentration of compound was 50 μM and the DMSO was 2%). A 1 μL aliquot of the inhibitor dilution series was spotted in a polypropylene 384-well V-bottom plate (Greiner) using a Thermo Scientific Matrix PlateMate (Thermo Scientific). The 100% inhibition control consisted of 1 mM final concentration of the product inhibitor S-adenosylhomocysteine (SAH, Sigma-Aldrich). Compounds were incubated for 30 minutes with 40 μL per well of 0.031 nM EHMT2 (recombinant purified human EHMT2 913-1193, Viva) in 1× assay buffer (20 mM Bicine [pH 7.5], 0.002% Tween 20, 0.005% Bovine Skin Gelatin and 1 mM TCEP). 10 μL per well of substrate mix comprising assay buffer, ³H-SAM (³H-labeled S-adenosylmethionine, American Radiolabeled Chemicals, specific activity of 80 Ci/mmol), unlabeled SAM (American Radiolabeled Chemicals), and peptide representing histone H3 residues 1-15 containing C-terminal biotin (appended to a C-terminal amide-capped lysine, synthesized by Biopeptide and HPLC-purified to greater than 95% purity) were added to initiate the reaction (both substrates were present in the final reaction mixture at their respective Kₘ values, an assay format referred to as “balanced conditions”). Reactions were incubated for 60 minutes at room temperature and quenched with 10 μL per well of 400 μM unlabeled SAM, then transferred to a 384-well streptavidin Flashplate (PerkinElmer) and washed in a Biotek ELx-405 well washer with 0.1% Tween after 60 minutes. 384-well Flashplates were read on a TopCount microplate reader (PerkinElmer).

General Procedure for MDA-MAB-231 HEK9me2 in-Cell Western Assay.

Compound (100 nL) was added directly to 384-well cell plate. MDA-MB-231 cells (ATCC) were seeded in assay medium (RPMI/Glutamax supplemented with 10% v/v heat inactivated fetal bovine serum and 1% Penicillin/Streptomycin, Life Technologies) at a concentration of 3,000 cells per well to a Poly-D-Lysine coated 384-well cell culture plate with 50 μL per well. Plates were incubated at 37° C., 5% CO₂ for 48 hours (BD Biosciences 356697). Plates were incubated at room temperature for 30 minutes and then incubated at 37° C., 5% CO₂ for additional 48 hours. After the incubation, 50 μL per well of 8% paraformaldehyde (Electron Microscopy Sciences) in PBS was added to the plates and incubated at room temperature for 20 minutes. Plates were transferred to a Biotek 406 plate washer and washed 2 times with 100 L per well of wash buffer (IX PBS containing 0.3% Triton X-100 (v/v)). Next, 60 μL per well of Odyssey blocking buffer (Licor Biosciences) was added to each plate and incubated for 1 hour at room temperature. Blocking buffer was removed and 20 μL of monoclonal primary antibody α-H3K₉me2 (Abcam) diluted 1:800 in Odyssey buffer with 0.1% Tween 20 (v/v) were added and plates were incubated overnight (16 hours) at 4° C. Plates were washed 5 times with 100 μL per well of wash buffer. Next 20 μL per well of secondary antibody was added (1:500 800CW donkey anti-mouse IgG (H+L) antibody (Licor Biosciences), 1:1000 DRAQ5 (Cell Signaling Technology) in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on a Licor Odyssey Infrared Scanner (Licor Biosciences) which measured integrated intensity at 700 nm and 800 nm wavelengths. Both 700 and 800 channels were scanned.

### % Inhibition Calculation.

First, the ratio for each well was determined by:

$$( \frac{H 3 K 9 me 2 \; 800 nm value}{DRAQ 5 \; 700 nm value} ) .$$

Each plate included fourteen control wells of DMSO only treatment (Minimum Inhibition) as well as fourteen control wells (background wells) for maximum inhibition treated with control compound UNC0638 (Background wells).

The average of the ratio values for each well was calculated and used to determine the percent inhibition for each test well in the plate. Control compound was serially diluted three-fold in DMSO for a total of 10 test concentrations beginning at 1 μM. Percent inhibition was calculated as

$$Percent Inhibition = 100 - ( ( \frac{( Individual Test Sample Ratio ) - ( Background Avg Ratio )}{( Minimum Inhibition Ratio ) - ( Background Average Ratio )} ) * 100 )$$

IC₅₀ curves were generated using triplicate wells per concentration of compound. The IC₅₀ is the concentration of compound at which measured methylation is inhibited by 50% as interpolated from the dose response curves. IC₅₀ values were calculated using a non-linear regression (variable slope-four parameter fit model) with by the following formula:

$$\% inhibition = Bottom + ( \frac{Top - Bottom}{( 1 + ( {IC}_{50} / {[ I ]}^{n} )} ) ,$$

where Top is fixed at 100% and Bottom is fixed to 0%, [I]=concentration of inhibitor, IC₅₀=half maximal inhibitory concentration and n=Hill Slope.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A compound being selected from 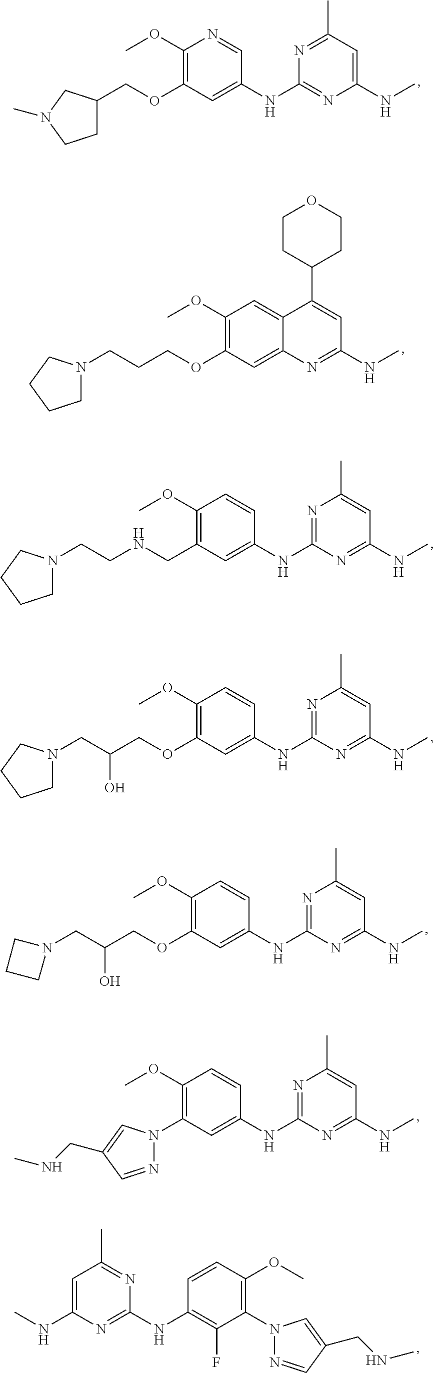
tautomers thereof, pharmaceutically acceptable salts thereof, and pharmaceutically acceptable salts of the tautomers.

2. The compound of claim 1, being selected from:

| Compound No. | Structure |
|---|---|
| 1 | 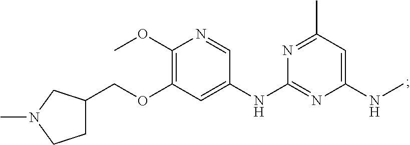 |
| 1R | 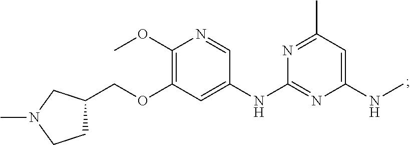 |
| 1S | 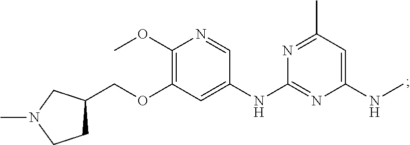 |
| 2 | 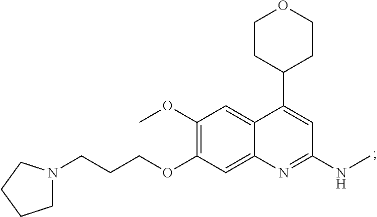 [SMILES: CNc1cc(C2CCOCC2)c2cc(OC)c(OCCCN3CCCC3)cc2n1] |
| 3 | 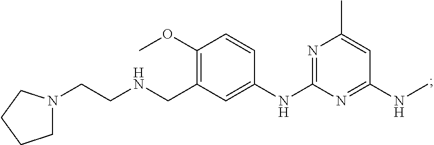 [SMILES: CNc1cc(C)nc(Nc2ccc(OC)c(CNCCN3CCCC3)c2)n1] |
| 4 | 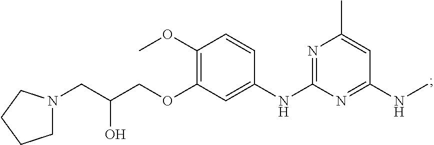 [SMILES: CNc1cc(C)nc(Nc2ccc(OC)c(OCC(O)CN3CCCC3)c2)n1] |
| 4R | 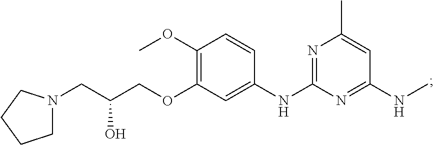 [SMILES: CNc1cc(C)nc(Nc2ccc(OC)c(OC[C@H](O)CN3CCCC3)c2)n1] |
| 4S | 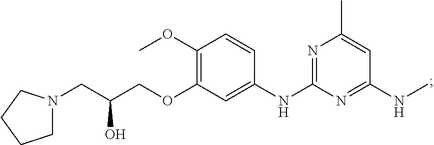 [SMILES: CNc1cc(C)nc(Nc2ccc(OC)c(OC[C@@H](O)CN3CCCC3)c2)n1] |
| 5 | 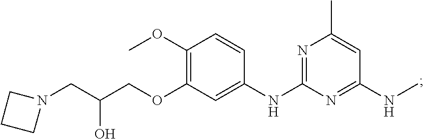 [SMILES: CNc1cc(C)nc(Nc2ccc(OC)c(OCC(O)CN3CCC3)c2)n1] |
| 5R | 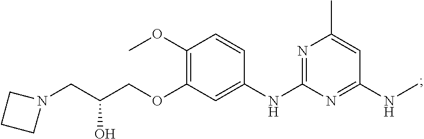 [SMILES: CNc1cc(C)nc(Nc2ccc(OC)c(OC[C@H](O)CN3CCC3)c2)n1] |
| 5S | 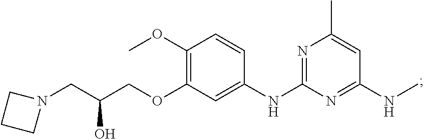 [SMILES: CNc1cc(C)nc(Nc2ccc(OC)c(OC[C@@H](O)CN3CCC3)c2)n1] |
| 6 | 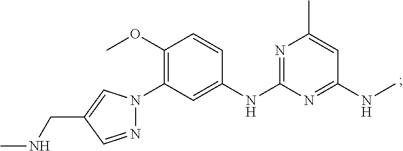 |
| 7 | 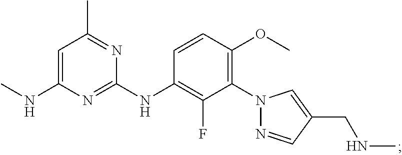 |

tautomers thereof, pharmaceutically acceptable salts thereof, and pharmaceutically acceptable salts of the tautomers.

3. The compound of claim 1 or claim 2, being 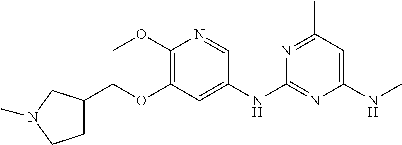
(Compound 1), a tautomer thereof, a pharmaceutically acceptable salt thereof, or a pharmaceutically acceptable salt of the tautomer.

4. The compound of any one of the preceding claims, being 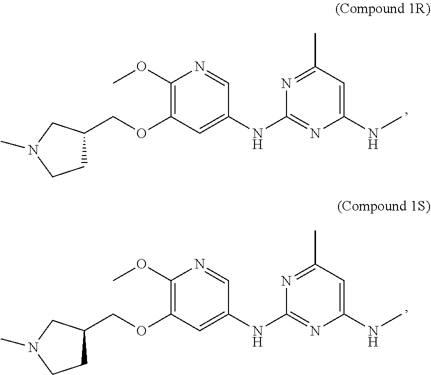
a tautomer thereof, a pharmaceutically acceptable salt thereof, or a pharmaceutically acceptable salt of the tautomer.

5. The compound of any one of the preceding claims, being Compound 1R, a tautomer thereof, a pharmaceutically acceptable salt thereof, or a pharmaceutically acceptable salt of the tautomer.

6. The compound of any one of the preceding claims, being Compound 1R.

7. The compound of any one of the preceding claims, being a crystalline form of Compound 1R.

8. The compound of any one of the preceding claims, being a pharmaceutical salt of Compound 1R.

9. The compound of any one of the preceding claims, being a hydrochloride salt of Compound 1R.

10. The compound of any one of the preceding claims, being a crystalline form of a hydrochloride salt of Compound 1R.

11. The compound of claim 1 or claim 2, being 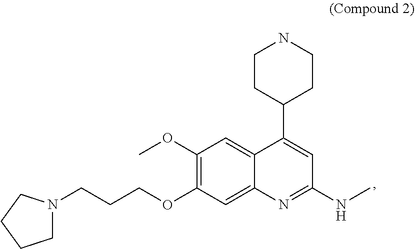 [SMILES: CNc1cc(C2CCNCC2)c2cc(OC)c(OCCCN3CCCC3)cc2n1]
a tautomer thereof, a pharmaceutically acceptable salt thereof, or a pharmaceutically acceptable salt of the tautomer.

12. The compound of any one of the preceding claims, being Compound 2.

13. The compound of any one of the preceding claims, being a crystalline form of Compound 2.

14. The compound of any one of the preceding claims, being a pharmaceutical salt of Compound 2.

15. The compound of any one of the preceding claims, being a hydrochloride salt of Compound 2.

16. The compound of any one of the preceding claims, being a crystalline form of a hydrochloride salt of Compound 2.

17. The compound of claim 1 or claim 3, being 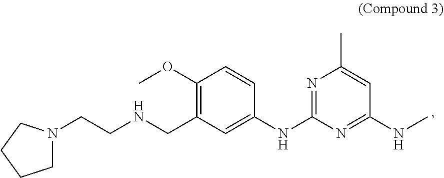 [SMILES: CNc1cc(C)nc(Nc2ccc(OC)c(CNCCN3CCCC3)c2)n1]
a tautomer thereof, a pharmaceutically acceptable salt thereof, or a pharmaceutically acceptable salt of the tautomer.

18. The compound of any one of the preceding claims, being Compound 3.

19. The compound of any one of the preceding claims, being a crystalline form of Compound 3.

20. The compound of any one of the preceding claims, being a pharmaceutical salt of Compound 3.

21. The compound of any one of the preceding claims, being a hydrochloride salt of Compound 3.

22. The compound of any one of the preceding claims, being a crystalline form of a hydrochloride salt of Compound 3.

23. The compound of any one of the preceding claims, being a sulfate salt of Compound 3.

24. The compound of any one of the preceding claims, being a crystalline form of a sulfate salt of Compound 3.

25. The compound of any one of the preceding claims, being a glycolate salt of Compound 3.

26. The compound of any one of the preceding claims, being a crystalline form of a glycolate salt of Compound 3.

27. The compound of any one of the preceding claims, being a succinate salt of Compound 3.

28. The compound of any one of the preceding claims, being a crystalline form of a succinate salt of Compound 3.

29. The compound of claim 1 or claim 2, being 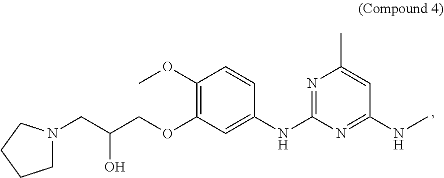 [SMILES: CNc1cc(C)nc(Nc2ccc(OC)c(OCC(O)CN3CCCC3)c2)n1]
a tautomer thereof, a pharmaceutically acceptable salt thereof, or a pharmaceutically acceptable salt of the tautomer.

30. The compound of any one of the preceding claims, being 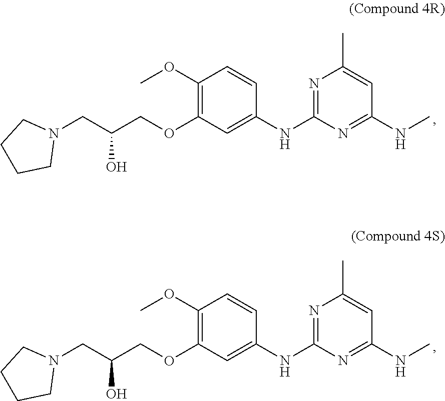 [SMILES: CNc1cc(C)nc(Nc2ccc(OC)c(OC[C@H](O)CN3CCCC3)c2)n1 ; CNc1cc(C)nc(Nc2ccc(OC)c(OC[C@@H](O)CN3CCCC3)c2)n1]
a tautomer thereof, a pharmaceutically acceptable salt thereof, or a pharmaceutically acceptable salt of the tautomer.

31. The compound of any one of the preceding claims, being Compound 4R.

32. The compound of any one of the preceding claims, being a crystalline form of Compound 4R.

33. The compound of any one of the preceding claims, being a pharmaceutical salt of Compound 4R.

34. The compound of any one of the preceding claims, being a hydrochloride salt of Compound 4R.

35. The compound of any one of the preceding claims, being a crystalline form of a hydrochloride salt of Compound 4R.

36. The compound of any one of the preceding claims, being a succinate salt of Compound 4R.

37. The compound of any one of the preceding claims, being a crystalline form of a succinate salt of Compound 4R.

38. The compound of claim 1 or claim 2, being 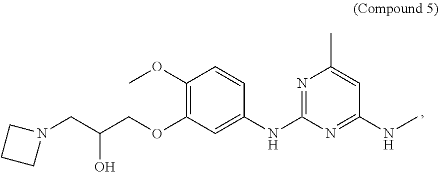 [SMILES: CNc1cc(C)nc(Nc2ccc(OC)c(OCC(O)CN3CCC3)c2)n1]
a tautomer thereof, a pharmaceutically acceptable salt thereof, or a pharmaceutically acceptable salt of the tautomer.

39. The compound of any one of the preceding claims, being 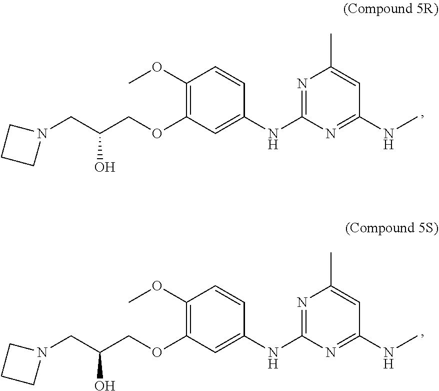 [SMILES: CNc1cc(C)nc(Nc2ccc(OC)c(OC[C@H](O)CN3CCC3)c2)n1 ; CNc1cc(C)nc(Nc2ccc(OC)c(OC[C@@H](O)CN3CCC3)c2)n1]
a tautomer thereof, a pharmaceutically acceptable salt thereof, or a pharmaceutically acceptable salt of the tautomer.

40. The compound of any one of the preceding claims, being Compound 5R.

41. The compound of any one of the preceding claims, being a crystalline form of Compound 5R.

42. The compound of any one of the preceding claims, being a pharmaceutical salt of Compound 5R.

43. The compound of any one of the preceding claims, being a sulfate salt of Compound 5R.

44. The compound of any one of the preceding claims, being a crystalline form of a sulfate salt of Compound 5R.

45. The compound of any one of the preceding claims, being a glycolate salt of Compound 5R.

46. The compound of any one of the preceding claims, being a crystalline form of a glycolate salt of Compound 5R.

47. The compound of any one of the preceding claims, being a fumarate salt of Compound 5R.

48. The compound of any one of the preceding claims, being a crystalline form of a fumarate salt of Compound 5R.

49. The compound of any one of the preceding claims, being a hippurate salt of Compound 5R.

50. The compound of any one of the preceding claims, being a crystalline form of a hippurate salt of Compound 5R.

51. The compound of any one of the preceding claims, being an adipate salt of Compound 5R.

52. The compound of any one of the preceding claims, being a crystalline form of an adipate salt of Compound 5R.

53. The compound of any one of the preceding claims, being a gentisate salt of Compound 5R.

54. The compound of any one of the preceding claims, being a crystalline form of a gentisate salt of Compound 5R.

55. The compound of any one of the preceding claims, being a benzoate salt of Compound 5R.

56. The compound of any one of the preceding claims, being a crystalline form of a benzoate salt of Compound 5R.

57. The compound of claim 1 or claim 2, being 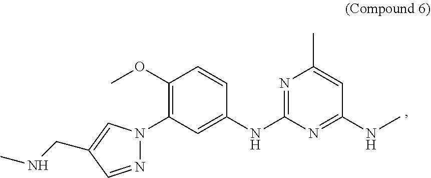
a tautomer thereof, a pharmaceutically acceptable salt thereof, or a pharmaceutically acceptable salt of the tautomer.

58. The compound of any one of the preceding claims, being a pharmaceutical salt of Compound 6.

59. The compound of any one of the preceding claims, being a hydrochloride salt of Compound 6.

60. The compound of any one of the preceding claims, being a crystalline form of a hydrochloride salt of Compound 6.

61. The compound of any one of the preceding claims, being a glycolate salt of Compound 6.

62. The compound of any one of the preceding claims, being a crystalline form of a glycolate salt of Compound 6.

63. The compound of any one of the preceding claims, being an adipate salt of Compound 6.

64. The compound of any one of the preceding claims, being a crystalline form of an adipate salt of Compound 6.

65. The compound of claim 1 or claim 2, being 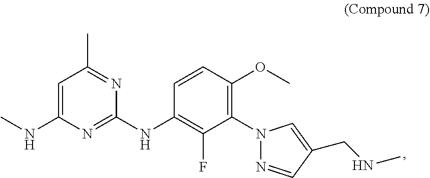
a tautomer thereof, a pharmaceutically acceptable salt thereof, or a pharmaceutically acceptable salt of the tautomer.

66. The compound of any one of the preceding claims, being a pharmaceutical salt of Compound 7.

67. The compound of any one of the preceding claims, being a hydrochloride salt of Compound 7.

68. The compound of any one of the preceding claims, being a crystalline form of a hydrochloride salt of Compound 7.

69. The compound of any one of the preceding claims, being an oxalate salt of Compound 7.

70. The compound of any one of the preceding claims, being a crystalline form of an oxalate salt of Compound 7.

71. The compound of any one of the preceding claims, being a sulfate salt of Compound 7.

72. The compound of any one of the preceding claims, being a crystalline form of a sulfate salt of Compound 7.

73. The compound of any one of the preceding claims, being a phosphate salt of Compound 7.

74. The compound of any one of the preceding claims, being a crystalline form of a phosphate salt of Compound 7.

75. The compound of any one of the preceding claims, being a fumarate salt of Compound 7.

76. The compound of any one of the preceding claims, being a crystalline form of a fumarate salt of Compound 7.

77. A pharmaceutical composition comprising the compound of any one of the preceding claims and a pharmaceutically acceptable carrier.

78. A method of inhibiting one or both of EHMT1 and EHMT2, the method comprising administering to a subject in need thereof a therapeutically effective amount of the compound of any one of the preceding claims.

79. The method of claim 49, wherein the subject has an EHMT-mediated disorder.

80. The method of claim 49, wherein the subject has a blood disorder.

81. The method of claim 49, wherein the subject has a cancer.

82. A method of preventing or treating a blood disorder, the method comprising administering to a subject in need thereof a therapeutically effective amount of the compound of any one of the preceding claims.

83. The method of claim 54, wherein the blood disorder is sickle cell anemia or R-thalassemia.

84. The method of claim 54, wherein the blood disorder is a hematological cancer.

85. A method of preventing or treating a cancer, the method comprising administering to a subject in need thereof a therapeutically effective amount of the compound of any one of the preceding claims.

86. The method of claim 56, wherein the cancer is lymphoma, leukemia, melanoma, breast cancer, ovarian cancer, hepatocellular carcinoma, prostate carcinoma, lung cancer, brain cancer, or hematological cancer.

87. The method of claim 56, wherein the cancer is melanoma.

88. The method of claim 57, wherein the hematological cancer is acute myeloid leukemia (AML) or chronic lymphocytic leukemia (CLL).

89. The method of claim 57, wherein the lymphoma is diffuse large B-cell lymphoma, follicular lymphoma, Burkitt's lymphoma or Non-Hodgkin's Lymphoma.

90. The method of claim 56, wherein the cancer is chronic myelogenous leukemia (CML), acute myeloid leukemia (AML), acute lymphocytic leukemia (ALL), mixed lineage leukemia (MLL), or myelodysplastic syndromes (MDS).

91. The compound of any one of the preceding claims for use in inhibiting one or both of EHMT1 and EHMT2 in a subject in need thereof.

92. The compound of any one of the preceding claims for use in preventing or treating an EHMT-mediated disorder in a subject in need thereof.

93. The compound of any one of the preceding claims for use in preventing or treating a blood disorder in a subject in need thereof.

94. The compound of any one of the preceding claims for use in preventing or treating a cancer in a subject in need thereof.

95. Use of the compound of any one of the preceding claims in the manufacture of a medicament for inhibiting one or both of EHMT1 and EHMT2 in a subject in need thereof.

96. Use of the compound of any one of the preceding claims in the manufacture of a medicament for preventing or treating an EHMT-mediated disorder in a subject in need thereof.

97. Use of the compound of any one of the preceding claims in the manufacture of a medicament for preventing or treating a blood disorder in a subject in need thereof.

98. Use of the compound of any one of the preceding claims in the manufacture of a medicament for preventing or treating a cancer in a subject in need thereof.
